SEQUENCE LISTING (171 sequences)

SEQ ID NO: 1 - DNA, 3903 bp, F. novicida U112
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 2 - protein, 1300 aa, F. novicida U112
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 1253 aa, Prevotella albensis M384
MNIKNFTGLYPLSKTLRFELKPIGKTKENIEKNGILTKDEQRAKDYLIVKGFIDEYHKQFIKDRLWDFKLPLESEGEKNSLEEYQELYELTKRNDAQEADFTEIKDNLRSSITEQLTKSGSAYDRIFKKEFIREDLVNFLEDEKDKNIVKQFEDFTTYFTGFYENRKNMYSSEEKSTAIAYRLIHQNLPKFMDNMRSFAKIANSSVSEHFSDIYESWKEYLNVNSIEEIFQLDYFSETLTQPHIEVYNYIIGKKVLEDGTEIKGINEYVNLYNQQQKDKSKRLPFLVPLYKQILSDREKLSWIAEEFDSDKKMLSAITESYNHLHNVLMGNENESLRNLLLNIKDYNLEKINITNDLSLTEISQNLFGRYDVFTNGIKNKLRVLTPRKKKETDENFEDRINKIFKTQKSFSIAFLNKLPQPEMEDGKPRNIEDYFITQGAINTKSIQKEDIFAQIENAYEDAQVFLQIKDTDNKLSQNKTAVEKIKTLLDALKELQHFIKPLLGSGEENEKDELFYGSFLAIWDELDTITPLYNKVRNWLTRKPYSTEKIKLNFDNAQLLGGWDVNKEHDCAGILLRKNDSYYLGIINKKTNHIFDTDITPSDGECYDKIDYKLLPGANKMLPKVFFSKSRIKEFEPSEAIINCYKKGTHKKGKNFNLTDCHRLINFFKTSIEKHEDWSKFGFKFSDTETYEDISGFYREVEQQGYRLTSHPVSASYIHSLVKEGKLYLFQIWNKDFSQFSKGTPNLHTLYWKMLFDKRNLSDVVYKLNGQAEVFYRKSSIEHQNRIIHPAQHPITNKNELNKKHTSTFKYDIIKDRRYTVDKFQFHVPITINFKATGQNNINPIVQEVIRQNGITHIIGIDRGERHLLYLSLIDLKGNIIKQMTLNEIINEYKGVTYKTNYHNLLEKREKERTEARHSWSSIESIKELKDGYMSQVIHKITDMMVKYNAIVVLEDLNGGFMRGRQKVEKQVYQKFEKKLIDKLNYLVDKKLDANEVGGVLNAYQLTNKFESFKKIGKQSGFLFYIPAWNTSKIDPITGFVNLFNTRYESIKETKVFWSKFDIIRYNKEKNWFEFVFDYNTFTTKAEGTRTKWTLCTHGTRIQTFRNPEKNAQWDNKEINLTESFKALFEKYKIDITSNLKESIMQETEKKFFQELHNLLHLTLQMRNSVTGTDIDYLISPVADEDGNFYDSRINGKNFPENADANGAYNIARKGLMLIRQIKQADPQKKFKFETITNKDWLKFAQDKPYLKD

SEQ ID NO: 4 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 5 - protein, 1282 aa, Eubacterium eligens CAG72
MNGNRSIVYREFVGVTPVAKTLRNELRPVGHTQEHIIQNGLIQEDELRQEKSTELKNIMDDYYREYIDKSLSGLTDLDFTLLFELMNSVQSSLSKDNKKALEKEHNKMREQICTHLQSDSDYKNMFNAKLFKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEIKKRLDMYMNMYHWVKAFIVDEVLDRDEMFYSDIDDIYNILENIVPLYNRVRNYVTQKPYTSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKNIVIKLNGQAELFYRKASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDVRIDMEKMYEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1231 aa, Butyrivibrio fibrisolvens
MYYESLTKLYPIKKTIRNELVPIGKTLENIKKNNILEADEDRKIAYIRVKAIMDDYHKRLINEALSGFALIDLDKAANLYLSRSKSADDIESFSRFQDKLRKAIAKRLREHENFGKIGNKDIIPLLQKLSENEDDYNALESFKNFYTYFESYNDVRLNLYSDKEKSSTVAYRLINENLPRFLDNIRAYDAVQKAGITSEELSSEAQDGLFLVNTFNNVLIQDGINTYNEDIGKLNVAINLYNQKNASVQGFRKVPKMKVLYKQILSDREESFIDEFESDTELLDSLESHYANLAKYFGSNKVQLLFTALRESKGVNVYVKNDIAKTSFSNVVFGSWSRIDELINGEYDDNNNRKKDEKYYDKRQKELKKNKSYTIEKIITLSTEDVDVIGKYIEKLESDIDDIRFKGKNFYEAVLCGHDRSKKLSKNKGAVEAIKGYLDSVKDFERDLKLINGSGQELEKNLVVYGEQEAVLSELSGIDSLYNMTRNYLTKKPFSTEKIKLNFNKPTFLDGWDYGNEEAYLGFFMIKEGNYFLAVMDANWNKEFRNIPSVDKSDCYKKVIYKQISSPEKSIQNLMVIDGKTVKKNGRKEKEGIHSGENLILEELKNTYLPKKINDIRKRRSYLNGDTFSKKDLTEFIGYYKQRVIEYYNGYSFYFKSDDDYASFKEFQEDVGRQAYQISYVDVPVSFVDDLINSGKLYLFRVYNKDFSEYSKGRLNLHTLYFKMLFDERNLKNVVYKLNGQAEVFYRPSSIKKEELIVHRAGEEIKNKNPKRAAQKPTRRLDYDIVKDRRYSQDKFMLHTSIIMNFGAEENVSFNDIVNGVLRNEDKVNVIGIDRGERNLLYVVVIDPEGKILEQRSLNCITDSNLDIETDYHRLLDEKESDRKIARRDWTTIENIKELKAGYLSQVVHIVAELVLKYNAIICLEDLNFGFKRGRQKVEKQVYQKFEKMLIDKLNYLVMDKSREQLSPEKISGALNALQLTPDFKSFKVLGKQTGIIYYVPAYLTSKIDPMTGFANLFYVKYENVDKAKEFFSKFDSIKYNKDGKNWNTKGYFEFAFDYKKFTDRAYGRVSEWTVCTVGERIIKFKNKEKNNSYDDKVIDLTNSLKELFDSYKVTYESEVDLKDAILAIDDPAFYRDLTRRLQQTLQMRNSSCDGSRDYIISPVKNSKGEFFCSDNNDDTTPNDADANGAFNIARKGLWVLNEIRNSEEGSKINLAMSNAQWLEYAQDNTI

SEQ ID NO: 7 - protein, 1250 aa, Smithella sp. SCADC
MQTLFENFTNQYPVSKTLRFELIPQGKTKDFIEQKGLLKKDEDRAEKYKKVKNIIDEYHKDFIEKSLNGLKLDGLEEYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKIYFRSGTSLTDVSRKVFGEWSIINRALDNYYATTYPIKPREKSEKYEERKEKWLKQDFNVSLIQTAIDEYDNETVKGKNSGKVIVDYFAKFCDDKETDLIQKVNEGYIAVKDLLNTPYPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKENLYYLGIMDKRHNRIFRNVPKADKKDSCYEKMVYKLLPGANKMLPKVFFSQSRIQEFTPSAKLLENYENETHKKGDNFNLNHCHQLIDFFKDSINKHEDWKNFDFRFSATSTYADLSGFYHEVEHQGYKISFQSIADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHVPITMNFKAEGIFNMNQRVNQFLKANPDINIIGIDRGERHLLYYTLINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLKQAKDFFEKFDSIRLNSKADYFEFAFDFKNFTGKADGGRTKWTVCTTNEDRYAWNRALNNNRGSQEKYDITAELKSLFDGKVDYKSGKDLKQQIASQELADFFRTLMKYLSVTLSLRHNNGEKGETEQDYILSPVADSMGKFFDSRKAGDDMPKNADANGAYHIALKGLWCLEQISKTDDLKKVKLAISNKEWLEFMQTLKG

SEQ ID NO: 8 - protein, 1273 aa, Flavobacterium sp. 316
MKNFSNLYQVSKTVRFELKPIGNTLENIKNKSLLKNDSIRAESYQKMKKTIDEFHKYFIDLALNNKKLSYLNEYIALYTQSAEAKKEDKFKADFKKVQDNLRKEIVSSFTEGEAKAIFSVLDKKELITIELEKWKNENNLAVYLDESFKSFTTYFTGFHQNRKNMYSAEANSTAIAYRLIHENLPKFIENSKAFEKSSQIAELQPKIEKLYKEFEAYLNVNSISELFEIDYFNEVLTQKGITVYNNIIGGRTATEGKQKIQGLNEIINLYNQTKPKNERLPKLKQLYKQILSDRISLSFLPDAFTEGKQVLKAVFEFYKINLLSYKQDGVEESQNLLELIQQVVKNLGNQDVNKIYLKNDTSLTTIAQQLFGDFSVFSAALQYRYETVVNPKYTAEYQKANEAKQEKLDKEKIKFVKQDYFSIAFLQEVVADYVKTLDENLDWKQKYTPSCIADYFTTHFIAKKENEADKTFNFIANIKAKYQCIQGILEQADDYEDELKQDQKLIDNIKFFLDAILEVVHFIKPLHLKSESITEKDNAFYDVFENYYEALNVVTPLYNMVRNYVTQKPYSTEKIKLNFENAQLLNGWDANKEKDYLTTILKRDGNYFLAIMDKKHNKTFQQFTEDDENYEKIVYKLLPGVNKMLPKVFFSNKNIAFFNPSKEILDNYKNNTHKKGATFNLKDCHALIDFFKDSLNKHEDWKYFDFQFSETKTYQDLSGFYKEVEHQGYKINFKKVSVSQIDTLIEEGKMYLFQIYNKDFSPYAKGKPNMHTLYWKALFETQNLENVIYKLNGQAEIFFRKASIKKKNIITHKAHQPIAAKNPLTPTAKNTFAYDLIKDKRYTVDKFQFHVPITMNFKATGNSYINQDVLAYLKDNPEVNIIGLDRGERHLVYLTLIDQKGTILLQESLNVIQDEKTHTPYHTLLDNKEIARDKARKNWGSIESIKELKEGYISQVVHKITKMMIEHNAIVVMEDLNFGFKRGRFKVEKQIYQKLEKMLIDKLNYLVLKDKQPHELGGLYNALQLTNKFESFQKMGKQSGFLFYVPAWNTSKIDPTTGFVNYFYTKYENVEKAKTFFSKFDSILYNKTKGYFEFVVKNYSDFNPKAADTRQEWTICTHGERIETKRQKEQNNNFVSTTIQLTEQFVNFFEKVGLDLSKELKTQLIAQNEKSFFEELFHLLKLTLQMRNSESHTEIDYLISPVANEKGIFYDSRKATASLPIDADANGAYHIAKKGLWIMEQINKTNSEDDLKKVKLAISNREWLQYVQQVQKK

SEQ ID NO: 9 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 10 - protein, 1262 aa, Bacteroidetes oral taxon 274
MRKFNEFVGLYPISKTLRFELKPIGKTLEHIQRNKLLEHDAVRADDYVKVKKIIDKYHKCLIDEALSGFTFDTEADGRSNNSLSEYYLYYNLKKRNEQEQKTFKTIQNNLRKQIVNKLTQSEKYKRIDKKELITTDLPDFLTNESEKELVEKFKNFTTYFTEFHKNRKNMYSKEEKSTAIAFRLINENLPKFVDNIAAFEKVVSSPLAEKINALYEDFKEYLNVEEISRVFRLDYYDELLTQKQIDLYNAIVGGRTEEDNKIQIKGLNQYINEYNQQQTDRSNRLPKLKPLYKQILSDRESVSWLPPKFDSDKNLLIKIKECYDALSEKEKVFDKLESILKSLSTYDLSKIYISNDSQLSYISQKMFGRWDIISKAIREDCAKRNPQKSRESLEKFAERIDKKLKTIDSISIGDVDECLAQLGETYVKRVEDYFVAMGESEIDDEQTDTTSFKKNIEGAYESVKELLNNADNITDNNLMQDKGNVEKIKTLLDAIKDLQRFIKPLLGKGDEADKDGVFYGEFTSLWTKLDQVTPLYNMVRNYLTSKPYSTKKIKLNFENSTLMDGWDLNKEPDNTTVIFCKDGLYYLGIMGKKYNRVFVDREDLPHDGECYDKMEYKLLPGANKMLPKVFFSETGIQRFLPSEELLGKYERGTHKKGAGFDLGDCRALIDFFKKSIERHDDWKKFDFKFSDTSTYQDISEFYREVEQQGYKMSFRKVSVDYIKSLVEEGKLYLFQIYNKDFSAHSKGTPNMHTLYWKMLFDEENLKDVVYKLNGEAEVFFRKSSITVQSPTHPANSPIKNKNKDNQKKESKFEYDLIKDRRYTVDKFLFHVPITMNFKSVGGSNINQLVKRHIRSATDLHIIGIDRGERHLLYLTVIDSRGNIKEQFSLNEIVNEYNGNTYRTDYHELLDTREGERTEARRNWQTIQNIRELKEGYLSQVIHKISELAIKYNAVIVLEDLNFGFMRSRQKVEKQVYQKFEKMLIDKLNYLVDKKKPVAETGGLLRAYQLTGEFESFKTLGKQSGILFYVPAWNTSKIDPVTGFVNLFDTHYENIEKAKVFFDKFKSIRYNSDKDWFEFVVDDYTRFSPKAEGTRRDWTICTQGKRIQICRNHQRNNEWEGQEIDLTKAFKEHFEAYGVDISKDLREQINTQNKKEFFEELLRLLRLTLQMRNSMPSSDIDYLISPVANDTGCFFDSRKQAELKENAVLPMNADANGAYNIARKGLLAIRKMKQEENDSAKISLAISNKEWLKFAQTKPYLED

SEQ ID NO: 11 - RNA, 38 nt, Artificial Sequence
  pre-crRNA
gggucuaagaacuuuaaauaauuucuacuguuguacau

SEQ ID NO: 12 - DNA, 112 bp, Artificial Sequence
  crRNA
agctgataagtaaattaccatcaatagtttctggatataataatttaagattaaaaggtaattctatcttgttgagatctgagctttcttctatatgattaatatttgctac

SEQ ID NO: 13 - DNA, 112 bp, Artificial Sequence
  crRNA
agctgtagcaaatattaatcatatagaagaaagctcagatctcaacaagatagaattaccttttaatcttaaattattatatccagaaactattgatggtaatttacttatc

SEQ ID NO: 14 - RNA, 38 nt, Artificial Sequence
  crRNA
uucuacugguguagauagauuaaaagguaauucuaucu

SEQ ID NO: 15 - DNA, 758 bp, Artificial Sequence
  CRISPR array
atgcgauucauagagaacaagagguuguuuuuauagacuaaaaauugcaaaccuuagucuuuauguuaaaauaacuacuaaguucuuagagauauuuaaaaauaugacuguuguuauauaucaaaaugcuaaaaaaaucauagauuuuaggucuuuuuuugcugauuuaggcaaaaacgggucuaagaacuuuaaauaauuucuacuguuguagaugagaagucauuuaauaaggccacuguuaaaagucuaagaacuuuaaauaauuucuacuguuguagaugcuacuauuccugugccuucagauaauucagucuaagaacuuuaaauaauuucuacuguuguagaugucuagagccuuuuguauuaguagccggucuaagaacuuuaaauaauuucuacuguuguagauuagcgauuuaugaaggucauuuuuuugucuaagaacuuuaaauaauuucuacuguuguagauagauuaaaagguaauucuaucuuguugaggucuaagaacuuuaaauaauuucuacuguuguagauuaccuaguagauacgcuuacugauaacaagucuaagaacuuuaaauaauuucuacuguuguagauaaacuuucauuuaugauauaaaguuuuuugucuaagaacuuuaaauaauuucuacuguuguagauucaaaaggcaagagagacggaaauaaauggacgucuaagaacuuuaaauaauuucuacuguuguagauuuguuugauugcuugcauuga

SEQ ID NO: 16 - RNA, 25 nt, Artificial Sequence
  Cpf1 cleavage product
gguagauuaaaagguaauucuaucu

SEQ ID NO: 17 - RNA, 40 nt, Artificial Sequence
  Cpf1 cleavage product
gguucuacuguuguagauagauuaaaagguaauucuaucu

SEQ ID NO: 18 - RNA, 67 nt, Artificial Sequence
  Cpf1 cleavage product
gggucuaagaacuuuaaauaauuucuacuguuguagauagauuaaaagguaauucuaucuuguugag

SEQ ID NO: 19 - RNA, 103 nt, Artificial Sequence
  Cpf1 cleavage product
gggucuaagaacuuuaaauaauuucuacuguuguagauagauuaaaagguaauucuaucuuguugaggucuaagaacuuuaaauaauuucuacuguuguagau

SEQ ID NO: 20 - RNA, 38 nt, Artificial Sequence
  pre-crRNA with spacer
gggucuaagaacuuuaaauaauuucuacuguuguagau

SEQ ID NO: 21 - RNA, 22 nt, Artificial Sequence
  pre-crRNA with spacer
aauaauuugaugaacacaucau

SEQ ID NO: 22 - RNA, 22 nt, Artificial Sequence
  pre-crRNA with spacer
aauaauuucuacuguucaucau

SEQ ID NO: 23 - RNA, 22 nt, Artificial Sequence
  pre-crRNA with spacer
aauuuaaucuacuguuguagau

SEQ ID NO: 24 - RNA, 20 nt, Artificial Sequence
  pre-crRNA with spacer
aauaauuucucuguugagau

SEQ ID NO: 25 - RNA, 24 nt, Artificial Sequence
  pre-crRNA with spacer
aauaauuucuacguguucguagau

SEQ ID NO: 26 - RNA, 30 nt, Artificial Sequence
  pre-crRNA with spacer
aauaauuucguguacguguucguacacgau

SEQ ID NO: 27 - RNA, 42 nt, Artificial Sequence
  crRNA
gguucuacuguuguagauuagcgauuuaugaaggucauuuuu

SEQ ID NO: 28 - RNA, 25 nt, Artificial Sequence
  crRNA
gguagauuaaaagguaauucuaucu

SEQ ID NO: 29 - RNA, 40 nt, Artificial Sequence; identical to SEQ ID NO: 17
  crRNA

SEQ ID NO: 30 - RNA, 67 nt, Artificial Sequence; identical to SEQ ID NO: 18
  crRNA

SEQ ID NO: 31 - RNA, 67 nt, Artificial Sequence
  crRNA
ggagauuaaaagguaauucuaucuuguugaggucuaagaacuuuaaauaauuucuacuguuguagau

SEQ ID NO: 32 - RNA, 103 nt, Artificial Sequence; identical to SEQ ID NO: 19
  crRNA

SEQ ID NO: 33 - RNA, 40 nt, Artificial Sequence
  crRNA
gguugaugaacacaucauagauuaaaagguaauucuaucu

SEQ ID NO: 34 - RNA, 40 nt, Artificial Sequence
  crRNA
gguucuacuguucaucauagauuaaaagguaauucuaucu

SEQ ID NO: 35 - DNA, 36 bp, Artificial Sequence
  non-target
atttaagattaaaaggtaattctatcttgttgagat

SEQ ID NO: 36 - DNA, 36 bp, Artificial Sequence
  target
atctcaacaagatagaattaccttttaatcttaaat

SEQ ID NO: 37 - RNA, 38 nt, Artificial Sequence; identical to SEQ ID NO: 14
  crRNA

SEQ ID NO: 38 - protein, 1300 aa, F. novicida U112; identical to SEQ ID NO: 2

SEQ ID NO: 39 - protein, 11 aa, HIV-1
YGRKKRRQRRR

SEQ ID NO: 40 - protein, 27 aa, Artificial Sequence
  Protein Transduction Domain
GWTLNSAGYLLGKINLKALAALAKKIL

SEQ ID NO: 41 - protein, 33 aa, Artificial Sequence
  Protein Transduction Domain
KALAWEAKLAKALAKALAKHLAKALAKALKCEA

SEQ ID NO: 42 - protein, 16 aa, Artificial Sequence
  Protein Transduction Domain
RQIKIWFQNRRMKWKK

SEQ ID NO: 43 - protein, 11 aa, Artificial Sequence
  Protein Transduction Domain
YGRKKRRQRRR

SEQ ID NO: 44 - protein, 9 aa, Artificial Sequence
  Protein Transduction Domain
RKKRRQRRR

SEQ ID NO: 45 - protein, 11 aa, Artificial Sequence
  Protein Transduction Domain
YGRKKRRQRRR

SEQ ID NO: 46 - protein, 8 aa, Artificial Sequence
  Protein Transduction Domain
RKKRRQRR

SEQ ID NO: 47 - protein, 11 aa, Artificial Sequence
  Protein Transduction Domain
YARAAARQARA

SEQ ID NO: 48 - protein, 11 aa, Artificial Sequence
  Protein Transduction Domain
THRLPRRRRRR

SEQ ID NO: 49 - protein, 11 aa, Artificial Sequence
  Protein Transduction Domain
GGRRARRRRRR

SEQ ID NO: 50 - protein, 16 aa, Drosophila melanogaster
RQIKIWFQNRRMKWKK

SEQ ID NO: 51 - DNA, 112 bp, Artificial Sequence; identical to SEQ ID NO: 13
  Primer

SEQ ID NO: 52 - DNA, 112 bp, Artificial Sequence; identical to SEQ ID NO: 12
  Primer

SEQ ID NO: 53 - DNA, 28 bp, Artificial Sequence
  Primer
agctgagatagaattaccttttaatctc

SEQ ID NO: 54 - DNA, 28 bp, Artificial Sequence
  Primer
agctgagattaaaaggtaattctatctc

SEQ ID NO: 55 - DNA, 24 bp, Artificial Sequence
  Primer
gacggccagtgcagtcgagctcgg

SEQ ID NO: 56 - DNA, 26 bp, Artificial Sequence
  Primer
ccttttaatctccgcttgcatgcctg

SEQ ID NO: 57 - DNA, 28 bp, Artificial Sequence
  Primer
agctgcgatagaattaccttttaatctc

SEQ ID NO: 58 - DNA, 28 bp, Artificial Sequence
  Primer
agctgagattaaaaggtaattctatcgc

SEQ ID NO: 59 - DNA, 28 bp, Artificial Sequence
  Primer
agctgatatagaattaccttttaatctc

SEQ ID NO: 60 - DNA, 28 bp, Artificial Sequence
  Primer
agctgagattaaaaggtaattctatatc

SEQ ID NO: 61 - DNA, 28 bp, Artificial Sequence
  Primer
agctgagctagaattaccttttaatctc

SEQ ID NO: 62 - DNA, 28 bp, Artificial Sequence
  Primer
agctgagattaaaaggtaattctagctc

SEQ ID NO: 63 - DNA, 28 bp, Artificial Sequence
  Primer
agctgagagagaattaccttttaatctc

SEQ ID NO: 64 - DNA, 28 bp, Artificial Sequence
  Primer
agctgagattaaaaggtaattctctctc

SEQ ID NO: 65 - DNA, 28 bp, Artificial Sequence
  Primer
agctgagatcgaattaccttttaatctc

SEQ ID NO: 66 - DNA, 28 bp, Artificial Sequence
  Primer
agctgagattaaaaggtaattcgatctc

SEQ ID NO: 67 - DNA, 28 bp, Artificial Sequence
  Primer
agctgagatataattaccttttaatctc

SEQ ID NO: 68 - DNA, 28 bp, Artificial Sequence
  Primer
agctgagattaaaaggtaattatatctc

SEQ ID NO: 69 - DNA, 28 bp, Artificial Sequence
  Primer
agctgagatagcattaccttttaatctc

SEQ ID NO: 70 - DNA, 28 bp, Artificial Sequence
  Primer
agctgagattaaaaggtaatgctatctc

SEQ ID NO: 71 - DNA, 28 bp, Artificial Sequence
  Primer
agctgagatagacttaccttttaatctc

SEQ ID NO: 72 - DNA, 28 bp, Artificial Sequence
  Primer
agctgagattaaaaggtaagtctatctc

SEQ ID NO: 73 - DNA, 28 bp, Artificial Sequence
  Primer
agctgagatagaagtaccttttaatctc

SEQ ID NO: 74 - DNA, 28 bp, Artificial Sequence
  Primer
agctgagattaaaaggtacttctatctc

SEQ ID NO: 75 - DNA, 28 bp, Artificial Sequence
  Primer
agctgagatagaatgaccttttaatctc

SEQ ID NO: 76 - DNA, 28 bp, Artificial Sequence
  Primer
agctgagattaaaaggtcattctatctc

SEQ ID NO: 77 - DNA, 28 bp, Artificial Sequence
  Primer
agctgagatagaattcccttttaatctc

SEQ ID NO: 78 - DNA, 28 bp, Artificial Sequence
  Primer
agctgagattaaaagggaattctatctc

SEQ ID NO: 79 - DNA, 28 bp, Artificial Sequence
  Primer
agctgagatagaattaacttttaatctc

SEQ ID NO: 80 - DNA, 28 bp, Artificial Sequence
  Primer
agctgagattaaaagttaattctatctc

SEQ ID NO: 81 - DNA, 28 bp, Artificial Sequence
  Primer
agctgagatagaattacattttaatctc

SEQ ID NO: 82 - DNA, 28 bp, Artificial Sequence
  Primer
agctgagattaaaatgtaattctatctc

SEQ ID NO: 83 - DNA, 28 bp, Artificial Sequence
  Primer
agctgagatagaattaccgtttaatctc

SEQ ID NO: 84 - DNA, 28 bp, Artificial Sequence
  Primer
agctgagattaaacggtaattctatctc

SEQ ID NO: 85 - DNA, 28 bp, Artificial Sequence
  Primer
agctgagatagaattacctgttaatctc

SEQ ID NO: 86 - DNA, 28 bp, Artificial Sequence
  Primer
agctgagattaacaggtaattctatctc

SEQ ID NO: 87 - DNA, 28 bp, Artificial Sequence
  Primer
agctgagatagaattaccttgtaatctc

SEQ ID NO: 88 - DNA, 28 bp, Artificial Sequence
  Primer
agctgagattacaaggtaattctatctc

SEQ ID NO: 89 - DNA, 28 bp, Artificial Sequence
  Primer
agctgagatagaattacctttgaatctc

SEQ ID NO: 90 - DNA, 28 bp, Artificial Sequence
  Primer
agctgagattcaaaggtaattctatctc

SEQ ID NO: 91 - DNA, 28 bp, Artificial Sequence
  Primer
agctgagatagaattaccttttcatctc

SEQ ID NO: 92 - DNA, 28 bp, Artificial Sequence
  Primer
agctgagatgaaaaggtaattctatctc

SEQ ID NO: 93 - DNA, 28 bp, Artificial Sequence
  Primer
agctgagatagaattaccttttactctc

SEQ ID NO: 94 - DNA, 28 bp, Artificial Sequence
  Primer
agctgagagtaaaaggtaattctatctc

SEQ ID NO: 95 - DNA, 28 bp, Artificial Sequence
  Primer
agctgagatagaattaccttttaagctc

SEQ ID NO: 96 - DNA, 28 bp, Artificial Sequence
  Primer
agctgagcttaaaaggtaattctatctc

SEQ ID NO: 97 - DNA, 28 bp, Artificial Sequence
  Primer
agctgagatagaattaccttttaatatc

SEQ ID NO: 98 - DNA, 28 bp, Artificial Sequence
  Primer
agctgatattaaaaggtaattctatctc

SEQ ID NO: 99 - DNA, 28 bp, Artificial Sequence
  Primer
agctgagatagaattaccttttaatcgc

SEQ ID NO: 100 - DNA, 28 bp, Artificial Sequence
  Primer
agctgcgattaaaaggtaattctatctc

SEQ ID NO: 101 - DNA, 28 bp, Artificial Sequence
  Primer
agctgctcgagaattaccttttaatctc

SEQ ID NO: 102 - DNA, 28 bp, Artificial Sequence
  Primer
agctgagattaaaaggtaattctcgagc

SEQ ID NO: 103 - DNA, 28 bp, Artificial Sequence
  Primer
agctgagatagaattaccttttacgagc

SEQ ID NO: 104 - DNA, 28 bp, Artificial Sequence
  Primer
agctgctcgtaaaaggtaattctatctc

SEQ ID NO: 105 - DNA, 24 bp, Artificial Sequence
  Primer
gacggccagtgcagtcgagctcgg

SEQ ID NO: 106 - DNA, 26 bp, Artificial Sequence
  Primer
ccttttaatctcatcttgcatgcctg

SEQ ID NO: 107 - DNA, 24 bp, Artificial Sequence
  Primer
gacggccagtgcagtcgagctcgg

SEQ ID NO: 108 - DNA, 26 bp, Artificial Sequence
  Primer
ccttttaatctcctctttcatgcctg

SEQ ID NO: 109 - DNA, 24 bp, Artificial Sequence
  Primer
gacggccagtgcagtcgagctcgg

SEQ ID NO: 110 - DNA, 26 bp, Artificial Sequence
  Primer
ccttttaatctcggcttgcatgcctg

SEQ ID NO: 111 - DNA, 92 bp, Artificial Sequence
  Primer
agctgtaatcatatagaagaaagctcagatctcaacaagatagaattaccttttaatcttaaattattatatccagaaactattgatggtac

SEQ ID NO: 112 - DNA, 92 bp, Artificial Sequence
  Primer
agctgtaccatcaatagtttctggatataataatttaagattaaaaggtaattctatcttgttgagatctgagctttcttctatatgattac

SEQ ID NO: 113 - DNA, 92 bp, Artificial Sequence
  Primer
agctgtaatcatatagaagaaagctcagatctcaacaagatagaattaccttttaatcttttatttttatatccagaaactattgatggtac

SEQ ID NO: 114 - DNA, 92 bp, Artificial Sequence
  Primer
agctgtaccatcaatagtttctggatataaaaataaaagattaaaaggtaattctatcttgttgagatctgagctttcttctatatgattac

SEQ ID NO: 115 - DNA, 92 bp, Artificial Sequence
  Primer
agctgtaatcatatagaagaaagctcagatctcaacaagatagaattaccttttaatcttttattattatatccagaaactattgatggtac

SEQ ID NO: 116 - DNA, 92 bp, Artificial Sequence
  Primer
agctgtaccatcaatagtttctggatataataataaaagattaaaaggtaattctatcttgttgagatctgagctttcttctatatgattac

SEQ ID NO: 117 - DNA, 92 bp, Artificial Sequence
  Primer
agctgtaatcatatagaagaaagctcagatctcaacaagatagaattaccttttaatcttggattattatatccagaaactattgatggtac

SEQ ID NO: 118 - DNA, 92 bp, Artificial Sequence
  Primer
agctgtaccatcaatagtttctggatataataatccaagattaaaaggtaattctatcttgttgagatctgagctttcttctatatgattac

SEQ ID NO: 119 - DNA, 112 bp, Artificial Sequence; identical to SEQ ID NO: 13
  Primer

SEQ ID NO: 120 - DNA, 112 bp, Artificial Sequence; identical to SEQ ID NO: 12
  Primer

SEQ ID NO: 121 - DNA, 17 bp, Artificial Sequence
  Primer
taatacgactcactata

SEQ ID NO: 122 - DNA, 59 bp, Artificial Sequence
  Primer
aaaaatgaccttcataaatcgctaatctacaacagtagaacctatagtgagtcgtatta

SEQ ID NO: 123 - DNA, 42 bp, Artificial Sequence
  Primer
agatagaattaccttttaatctacctatagtgagtcgtatta

SEQ ID NO: 124 - DNA, 57 bp, Artificial Sequence
  Primer
agatagaattaccttttaatctatctacaacagtagaacctatagtgagtcgtatta

SEQ ID NO: 125 - DNA, 84 bp, Artificial Sequence
  Primer
ctcaacaagatagaattaccttttaatctatctacaacagtagaaattatttaaagttcttagaccctatagtgagtcgtatta

SEQ ID NO: 126 - DNA, 84 bp, Artificial Sequence
  Primer
atctacaacagtagaaattatttaaagttcttagacctcaacaagatagaattaccttttaatctcctatagtgagtcgtatta

SEQ ID NO: 127 - DNA, 120 bp, Artificial Sequence
  Primer
atctacaacagtagaaattatttaaagttcttagacctcaacaagatagaattaccttttaatctatctacaacagtagaaattatttaaagttcttagaccctatagtgagtcgtatta

SEQ ID NO: 128 - DNA, 57 bp, Artificial Sequence
  Primer
agatagaattaccttttaatctatgatgaacagtagaacctatagtgagtcgtatta

SEQ ID NO: 129 - DNA, 57 bp, Artificial Sequence
  Primer
agatagaattaccttttaatctatgatgtgttcatcaacctatagtgagtcgtatta

SEQ ID NO: 130 - DNA, 84 bp, Artificial Sequence
  Primer
ctcaacaagatagaattaccttttaatctatgatgaacagtagaaattatttaaagttcttagaccctatagtgagtcgtatta

SEQ ID NO: 131 - DNA, 84 bp, Artificial Sequence; identical to SEQ ID NO: 130
  Primer

SEQ ID NO: 132 - DNA, 34 bp, Artificial Sequence
  Primer
atgcaggtcgacatgtcaatttatcaagaatttg

SEQ ID NO: 133 - DNA, 35 bp, Artificial Sequence
  Primer
agctagcggccgcttagttattcctattctgcacg

SEQ ID NO: 134 - DNA, 34 bp, Artificial Sequence
  Primer
atgcagggtaccatgtcaatttatcaagaatttg

SEQ ID NO: 135 - DNA, 33 bp, Artificial Sequence
  Primer
agctacggccgttagttattcctattctgcacg

SEQ ID NO: 136 - DNA, 49 bp, Artificial Sequence
  Primer
tcgtaaacaatcaatacctaaaaaaatcactgccccagctaaagaggca

SEQ ID NO: 137 - DNA, 49 bp, Artificial Sequence
  Primer
tgcctctttagctggggcagtgatttttttaggtattgattgtttacga

SEQ ID NO: 138 - DNA, 53 bp, Artificial Sequence
  Primer
ctcttttttaggattatctttgtttgcattagctattgcctctttagctgggt

SEQ ID NO: 139 - DNA, 53 bp, Artificial Sequence
  Primer
acccagctaaagaggcaatagctaatgcaaacaaagataatcctaaaaaagag

SEQ ID NO: 140 - DNA, 64 bp, Artificial Sequence
  Primer
gaaaaacttatcttcagtaaagcgtttatctgcgattaaatcatattcaaaaacactctctttt

SEQ ID NO: 141 - DNA, 64 bp, Artificial Sequence
  Primer
aaaagagagtgtttttgaatatgatttaatcgcagataaacgctttactgaagataagtttttc

SEQ ID NO: 142 - DNA, 62 bp, Artificial Sequence
  Primer
ggacagtgaaagaaaaacttatcttcagtagcgcgtttatctttgattaaatcatattcaaa

SEQ ID NO: 143 - DNA, 62 bp, Artificial Sequence
  Primer
tttgaatatgatttaatcaaagataaacgcgctactgaagataagtttttctttcactgtcc

SEQ ID NO: 144 - DNA, 53 bp, Artificial Sequence
  Primer
aagctaaatgtctttcacctctagctatacttaatatatgaacatcatttgct

SEQ ID NO: 145 - DNA, 53 bp, Artificial Sequence
  Primer
agcaaatgatgttcatatattaagtatagctagaggtgaaagacatttagctt

SEQ ID NO: 146 - DNA, 53 bp, Artificial Sequence
  Primer
tcatatattaagtatagatagaggtgcaagacatttagcttactatactttgg

SEQ ID NO: 147 - DNA, 53 bp, Artificial Sequence
  Primer
ccaaagtatagtaagctaaatgtcttgcacctctatctatacttaatatatga

SEQ ID NO: 148 - DNA, 56 bp, Artificial Sequence
  Primer
ccatctaccaaagtatagtaagctaaagctctttcacctctatctatacttaatat

SEQ ID NO: 149 - DNA, 56 bp, Artificial Sequence
  Primer
atattaagtatagatagaggtgaaagagctttagcttactatactttggtagatgg

SEQ ID NO: 150 - DNA, 50 bp, Artificial Sequence
  Primer
cctttaccatctaccaaagtataggcagctaaatgtctttcacctctatc

SEQ ID NO: 151 - DNA, 50 bp, Artificial Sequence
  Primer
gatagaggtgaaagacatttagctgcctatactttggtagatggtaaagg

SEQ ID NO: 152 - DNA, 59 bp, Artificial Sequence
  Primer
ctcttttaaatccaaaatttaaatccgcaaaaaccacaatagcattatactctataact

SEQ ID NO: 153 - DNA, 59 bp, Artificial Sequence
  Primer
agttatagagtataatgctattgtggtttttgcggatttaaattttggatttaaaagag

SEQ ID NO: 154 - DNA, 51 bp, Artificial Sequence
  Primer
aattagcattttttctaacttttgagcgacctgcttctctaccttgaaacg

SEQ ID NO: 155 - DNA, 51 bp, Artificial Sequence
  Primer
cgtttcaaggtagagaagcaggtcgctcaaaagttagaaaaaatgctaatt

SEQ ID NO: 156 - DNA, 51 bp, Artificial Sequence
  Primer
gtttagtttctcaattagcatttttgctaacttttgatagacctgcttctc

SEQ ID NO: 157 - DNA, 51 bp, Artificial Sequence
  Primer
gagaagcaggtctatcaaaagttagcaaaaatgctaattgagaaactaaac

SEQ ID NO: 158 - DNA, 47 bp, Artificial Sequence
  Primer
ctgctactggtgaaattagataagctaactcagtacctgtttttgag

SEQ ID NO: 159 - DNA, 47 bp, Artificial Sequence
  Primer
ctcaaaaacaggtactgagttagcttatctaatttcaccagtagcag

SEQ ID NO: 160 - DNA, 35 bp, Artificial Sequence
  Primer
gataagcaccattggcagcagcatcttgaggcata

SEQ ID NO: 161 - DNA, 35 bp, Artificial Sequence
  Primer
tatgcctcaagatgctgctgccaatggtgcttatc

SEQ ID NO: 162 - DNA, 29 bp, Artificial Sequence
  Primer
atcaagcccttcatgcgcttcaaggtgca

SEQ ID NO: 163 - DNA, 37 bp, Artificial Sequence
  Primer
agtttaggtaccttattttctccactctaaacttgat

SEQ ID NO: 164 - DNA, 47 bp, Artificial Sequence
  Primer
atattcaacatattgaccggcctgcagagtaaggatgttgggtctac

SEQ ID NO: 165 - DNA, 84 bp, Artificial Sequence; identical to SEQ ID NO: 130
  Primer

SEQ ID NO: 166 - DNA, 60 bp, Artificial Sequence
  Primer
atgggccatcatcatcatcatcatcatcatcatcacactacagtaaaaaaaaacagagcg

SEQ ID NO: 167 - DNA, 13 bp, Artificial Sequence
  Primer
ggtggtaaatttg

SEQ ID NO: 168 - DNA, 12 bp, Artificial Sequence
  Primer
gtcagtcagaag

SEQ ID NO: 169 - DNA, 24 bp, Artificial Sequence
  Primer
ggtttataagctaaatggtgaggc

SEQ ID NO: 170 - DNA, 18 bp, Artificial Sequence
  Primer
gtcgcgaacgccagcaag

SEQ ID NO: 171 - DNA, 180 bp, Artificial Sequence
  Primer
atgcagaagcttttgacagctagctcagtcctaggtataatgctagcgtctaagaactttaaataatttctactgttgtagattgcaccttgaagcgcatgaagggcttgatgtctaagaactttaaataatttgtctgtatattattgatttctaaattagaattttcggccgatgcag

(19) United States
(12) Patent Application Publication
(10) Pub. No.: US 2019/0048340 A1
(43) Pub. Date: Feb. 14, 2019

(54) NOVEL FAMILY OF RNA-PROGRAMMABLE ENDONUCLEASES AND THEIR USES IN GENOME EDITING AND OTHER APPLICATIONS

(71) Applicants: CRISPR Therapeutics AG, Basel (CH); Max-Planck-Gesellschaft zur Förderung der Wissenschaften e.V., Munich (DE); Helmholtz-Zentrum für Infektionsforschung GmbH, Braunschweig (DE)

(72) Inventors: Emmanuelle Marie Charpentier, Berlin (DE); Ines Fonfara, Berlin (DE); Ante Sven Lundberg, Cambridge, MA (US); Hagen Klaus Gunther Richter, Berlin (DE)

(21) Appl. No.: 15/760,739

(22) PCT Filed: Sep. 22, 2016
(86) PCT No.: PCT/IB2016/001418

§ 371 Date: Mar. 16, 2018

Related U.S. Application Data
(60) Provisional application No. 62/324,309, filed on Apr. 18, 2016.
(60) Provisional application No. 62/296,895, filed on Feb. 18, 2016.
(60) Provisional application No. 62/266,155, filed on Dec. 11, 2015.
(60) Provisional application No. 62/261,451, filed on Dec. 1, 2015.
(60) Provisional application No. 62/260,059, filed on Nov. 25, 2015.
(60) Provisional application No. 62/232,381, filed on Sep. 24, 2015.

(51) Int. Cl. C12N 15/113; C12N 9/22; C12N 15/10; C12N 15/63

(52) U.S. Cl. CPC C12N 15/113; C12N 9/22; C12N 2750/14141; C12N 15/63; C12N 2310/20; C12N 15/102

(57) ABSTRACT

A new family of RNA-programmable endonucleases, associated guide RNAs and target sequences, and their uses in genome editing and other applications are disclosed herein.

### CROSS REFERENCE TO RELATED APPLICATIONS

This application claims the benefit of U.S. Application No. 62/232,381, filed on Sep. 24, 2015, U.S. Application No. 62/260,059, filed on Nov. 25, 2015, U.S. Application No. 62/261,451, filed on Dec. 1, 2015, U.S. Application No. 62/266,155 filed on Dec. 11, 2015, U.S. Application No. 62/296,895, filed on Feb. 18, 2016, and U.S. Application No. 62/324,309, filed on Apr. 18, 2016. The disclosures of these related application are herein incorporated by reference in their entirety. To the extent that there are any discrepancies between the disclosures of these related applications and the instant application, the disclosure of the instant application should control.

### REFERENCE TO SEQUENCE LISTING

The instant application contains a Sequence Listing which has been submitted electronically in ASCII format and is hereby incorporated by reference in its entirety. Said ASCII copy, created on Sep. 21, 2016, is named 0116339_00007_Sequence_Listing.txt and is 148,808 bytes in size.

### FIELD

Disclosed herein is a new family of RNA-programmable endonucleases, associated guide RNAs and target sequences, and their uses in genome editing and other applications.

### BACKGROUND

Endonucleases such as Zinc-finger endonucleases (ZFNs), Transcription-activator like effector nucleases (TALENs) and ribonucleases have been harnessed as site-specific nucleases for genome targeting, genome editing, gene silencing, transcription modulation, promoting recombination and other molecular biological techniques. CRISPR-Cas systems provide a source of novel nucleases and endonucleases, including CRISPR-Cas9, which has already been developed into a powerful technology for genome targeting.

Editing genomes using the RNA-guided DNA targeting principle of CRISPR-Cas (Clustered Regularly Interspaced Short Palindromic Repeats-CRISPR associated proteins), as described in WO2013/176722, has been exploited widely over the past few years. Three types of CRISPR-Cas systems (type I, type II, and type III) have previously been described, and a fourth was more recently identified (type V). Most uses of CRISPR-Cas for genome editing have been with the type II system. The main advantage provided by the bacterial type II CRISPR-Cas system lies in the minimal requirement for programmable DNA interference: an endonuclease, Cas9, guided by a customizable dual-RNA structure. As initially demonstrated in the original type II system of Streptococcus pyogenes, trans-activating CRISPR RNA (tracrRNA) binds to the invariable repeats of precursor CRISPR RNA (pre-crRNA) forming a dual-RNA that is essential for both RNA co-maturation by RNase III in the presence of Cas9, and invading DNA cleavage by Cas9. As demonstrated in Streptococcus, Cas9 guided by the duplex formed between mature activating tracrRNA and targeting crRNA introduces site-specific double-stranded DNA (dsDNA) breaks in the invading cognate DNA. Cas9 is a multi-domain enzyme that uses an HNH nuclease domain to cleave the target strand (defined as complementary to the spacer sequence of crRNA) and a RuvC-like domain to cleave the non-target strand, enabling the conversion of the dsDNA cleaving Cas9 into a nickase by selective motif inactivation. DNA cleavage specificity is determined by two parameters: the variable, spacer-derived sequence of crRNA targeting the protospacer sequence (a protospacer is defined as the sequence on the DNA target that is complementary to the spacer of crRNA) and a short sequence, the Protospacer Adjacent Motif (PAM), located immediately downstream of the protospacer on the non-target DNA strand.

To date, RNA-guided Cas9 from multiple species have been described as tools for genome manipulation. Studies have demonstrated that RNA-guided Cas9 can be employed as an efficient genome editing tool in human cells, mice, zebrafish, drosophila, worms, plants, yeast and bacteria, as well as various other species. The system is versatile, enabling multiplex genome engineering by programming Cas9 to edit several sites in a genome simultaneously by simply using multiple guide RNAs. The conversion of Cas9 into a nickase was shown to facilitate homology-directed repair in mammalian genomes with reduced mutagenic activity. In addition, the DNA-binding activity of a Cas9 catalytic inactive mutant has been exploited to engineer RNA-programmable transcriptional silencing and activating devices.

Following the description of three main types of CRISPR-Cas, a fourth type was recently identified, and here we describe a new type of CRISPR-Cas endonuclease, referred to as a type V CRISPR-Cas. For clarity, this designation of CRISPR-Cas includes CRISPR-associated endonuclease Cpf1.

The present invention provides a novel family of CRISPR-Cas endonucleases having different characteristics and functionalities from known CRISPR-Cas endonucleases and thus provides further opportunities for genome editing that did not exist previously.

### SUMMARY

The invention relates to a new family of RNA-programmable endonucleases, associated guide RNAs and target sequences, and their uses in genome editing.

CRISPR-Cas adaptive immunity in bacteria and archaea involves a set of distinct proteins for production of mature CRISPR RNAs (crRNAs) and interference with invading nucleic acids. Cpf1 and its orthologs are a novel family of single enzyme CRISPR-associated proteins with dual-endoribonuclease-endonuclease activity in precursor crRNA (pre-crRNA) processing and crRNA-programmable cleavage of target DNA, which can be used in RNA-programmable genome editing.

Type V-A Cpf1 is a dual-nuclease in crRNA biogenesis and interference. Cpf1 cleaves pre-crRNA upstream of a hairpin structure formed within the repeats to generate first intermediate crRNAs that are processed further to mature crRNAs (both the pre-processed substrates and the processed substrate nucleic acids are referred to as “guide RNAs” or “gRNAs”). “GuideRNA” is a mature crRNA, or any artificially created pre-processed form thereof, capable of being processed in vitro or in vivo into a mature crRNA. Cpf1, guided by mature repeat-spacer crRNAs, introduces double-stranded breaks in target DNA generating a 5′ overhang. The RNA and DNA nucleolytic activities of Cpf1 require sequence- and structure-specific recognition of the hairpin of crRNA repeats. DNA cleavage by Cpf1 is dependent on the presence of a double-stranded 5′-NAR-3′ (N is any nucleotide; R is a purine base (G or A)) protospacer adjacent motif (PAM) on the target DNA strand (also defined as 5′-YTN-3′ (Y=T or C) upstream of the crRNA-complementary DNA sequence on the non-target strand (FIGS. 3D and 13C)). A seed sequence of eight nucleotides proximal to the PAM was determined. Cpf1 uses distinct active domains for both nuclease reactions and cleaves nucleic acids in the presence of magnesium or calcium. This represents a new family of enzymes with dual-endoribonuclease and endonuclease activities, and demonstrates that Type V-A constitutes the most minimal of the already described CRISPR-Cas systems. In addition, this new family of enzymes can be used for RNA-programmable genome editing. In one aspect, provided herein is a method for targeting, editing or manipulating DNA in vitro or in a cell comprising contacting the DNA with a heterologous Cpf1 polypeptide and a single heterologous nucleic acid comprising one or more pre-CRISPR RNAs (pre-crRNA), or intermediate or mature crRNAs, each RNA comprising a minimum of a repeat-spacer array in the 5′ to 3′ direction (including, for example, an array having a single set of repeat-spacer elements and spacer-repeat arrays), wherein the repeat comprises a stem-loop structure. In some embodiments, the heterologous nucleic acid is of a defined length, which is shorter than the corresponding guide RNA required for Cas9.

In another aspect, provided herein is a system for targeting, editing or manipulating DNA in a cell comprising a heterologous vector encoding or providing a Cpf1 polypeptide and a single heterologous nucleic acid comprising one or more pre-CRISPR RNAs (pre-crRNA), or intermediate or mature crRNAs, each RNA comprising a minimum of a repeat-spacer array in the 5′ to 3′ direction, wherein the repeat comprises a stem-loop structure.

Unless otherwise is noted or follows form the context, the term repeat-spacer array refers not only arrays comprising multiple repeat-spacer units but also to a single repeat-spacer unit.

In some embodiments, the Cpf1 polypeptide is a monomer. In some embodiments, the Cpf1 polypeptide has an apparent molecular weight of about 187 kDa. In some embodiments, the enzyme is a monomer when recombinantly expressed in the cell and/or after it is purified, for example, by Nickel-affinity or other suitable purification techniques.

In some embodiments, the Cpf1 polypeptide has an RNA cleavage domain and a DNA cleavage domain.

In some embodiments, the RNA cleavage domain of the Cpf1 polypeptide cleaves each of the one or more pre-crRNAs or intermediate crRNAs within the repeat of the repeat-spacer array and 4 nucleotides upstream of the stem-loop (FIGS. 2A-2B). The intermediate or pre-RNA can be cleaved or trimmed by other enzymes. In some embodiments, the RNA cleavage domain of the Cpf1 polypeptide cleaves each of the one or more pre-crRNAs or intermediate crRNAs four nucleotides upstream of the stem-loop structure. In some embodiments, the RNA cleavage domain of the Cpf1 polypeptide cleaves the one or more pre-crRNAs or intermediate crRNAs at a higher level of activity in the presence of Mg²⁺, and, at an even higher level, in the presence of Ca²⁺. Of note, some RNA processing without the divalent ions can be achieved, albeit with lower efficiency.

In some embodiments, the one or more pre-crRNAs or intermediate crRNAs are cleaved and processed into one or more mature crRNAs.

In some embodiments, the one or more mature crRNAs guides the DNA cleavage domain of the Cpf1 polypeptide.

In some embodiments, the DNA cleavage domain of the Cpf1 polypeptide is capable of cleaving the DNA in the presence of either Mg²⁺, Mn²⁺ or Ca²⁺. In certain embodiments, the Cpf1 polypeptide is capable of cleaving RNA in the presence of Mg²⁺ or, less preferably, Ca²⁺. In some embodiments, the DNA cleavage domain of the Cpf1 polypeptide cleaves the DNA via a staggered cut that produces a five nucleotide 5′ overhang. In some embodiments, the DNA cleavage domain of the Cpf1 polypeptide recognizes a PAM sequence in the DNA that is 5′-YTN-3′ (Y=T or C) upstream of the crRNA-complementary DNA sequence on the non-target strand, or 5′-NAR-3′ downstream of the crRNA-complementary DNA sequence of the target strand, specifically including the PAM sequence in the DNA that is 5′-NAG-3′ downstream of the crRNA-complementary DNA sequence of the target strand. In some embodiments, the DNA cleavage domain of the Cpf1 polypeptide has a seed sequence of eight nucleotides proximal to the PAM. In some embodiments, the DNA cleavage domain of the Cpf1 polypeptide cleaves the DNA about 20 nucleotides upstream of the PAM sequence. In some embodiments the Cpf1 polypeptide cleaves the DNA exactly 22 base pairs upstream of the PAM sequence on the crRNA-complementary target strand and 17 base pairs downstream of the PAM sequence on the non-crRNA-complementary non-target strand (FIG. 2). In another aspect, provided herein is a method for improved Cpf1 endonuclease activity in targeting, editing or manipulating DNA in vitro or in a cell by combining Cpf1 polypeptide, or a heterologous vector encoding Cpf1 or providing polypeptide, together with one or more heterologous nucleic acids comprising one or more pre-crRNAs or intermediate RNAs, wherein the improved activity is obtained by using a form of crRNA that is longer than the mature form of crRNA, for example, intermediate form of crRNA. As shown for example in FIG. 11, (cf. lanes 4 vs. 3 and 6), processing of the larger crRNA by Cpf1 may enhance DNA endonuclease activity of Cpf1 (FIG. 11, cf. lanes 4 vs. 3 and 6).

In another aspect, provided herein is a method for modulation of endoribonuclease activity in the absence of modulation of DNA endonuclease activity, and/or modulation of DNA endonuclease activity in the absence of modulation of endoribonuclease activity, or modulate nuclease activity in the presence or absence of specific divalent cations such as magnesium or calcium, and/or to modulate cleavage of only one, but not the other, DNA strand, and/or to modulate RNA stability or half life, and/or DNA binding by the Cpf1 endonuclease, by mutation or modification of specific amino acid residues in the Cpf1 polypeptide selected from the group consisting of: H843, K852, K869, F873, D917, E1006, D1255, E920, Y1024, D1227, E1028, H922, and Y925 (FIG. 3), for example, by substitution of any one of these amino acid residues with alanine (A).

In some embodiments, the Cpf1 polypeptide is a mutant polypeptide with altered Cpf1 endoribonuclease activity or associated half life of pre-crRNA, intermediate crRNA, or mature crRNA, and having one or more mutations at amino acid residues selected from the group consisting of: H843, K852, K869, and F873, for example, H843A, K852A, K869A, and F873A.

In some embodiments, the Cpf1 polypeptide is a mutant polypeptide with altered or abrogated DNA endonuclease activity without substantially diminished or enhanced endoribonuclease activity or binding affinity to DNA and having one or more mutations at amino acid residues selected from the group consisting of: D917, E1006, and D1255, for example, D917A, E1006A, and D1255A. Such modification can allow for the sequence specific DNA targeting of Cpf1 for the purpose of transcriptional modulation, activation, or repression; epigenetic modification or chromatin modification by methylation, demethylation, acetylation or deacetylation, or any other modifications of DNA binding proteins known in the art.

In some embodiments, the Cpf1 polypeptide is a mutant polypeptide with no DNA endonuclease activity in the presence of Ca²⁺, without substantially diminished or enhanced DNA endonuclease activity in the presence of Mg²⁺, and having one or more mutations at amino acid residues selected from the group consisting of: E920, Y1024, and D1227, for example, E920A, Y1024A, and D1227A.

In some embodiments, the Cpf1 polypeptide is a mutant polypeptide with no DNA endonuclease activity in the presence of Ca²⁺, and substantially reduced DNA endonuclease activity of the non-target strand in the presence of Mg²⁺, and having a mutation at amino acid residue E1028, for example, E1 028A.

In some embodiments, the Cpf1 polypeptide is a mutant polypeptide with substantially decreased DNA endonuclease activity of the target strand in the presence of Ca²⁺, without substantially diminished or enhanced DNA endonuclease activity in the presence of Mg²⁺, and having one or more mutations at amino acid residues selected from: H922 and Y925, for example, H922A and Y925A.

In some embodiments, the cell is a bacterial cell, a fungal cell, an archaea cell, a plant cell, or an animal cell.

In some embodiments, the Cpf1 polypeptide and the single heterologous nucleic acid are introduced into the cell by the same or different recombinant vectors encoding the polypeptide and the nucleic acid.

In some embodiments, the nucleic acid encoding the polypeptide, nucleic acid, or both the polypeptide and nucleic acid is modified.

In some embodiments, the method or system further comprises adding a donor DNA sequence, and wherein the target DNA sequence is edited by homology directed repair. In some embodiments, the polynucleotide donor template is physically linked to a crRNA or guide RNA.

In another aspect, provided herein is a method for modifying or editing double stranded DNA or single stranded target DNA, without having activity against ssRNA, dsRNA, or heteroduplexes of RNA and DNA.

In another aspect, provided herein is a method for editing or modifying DNA at multiple locations in a cell consisting essentially of: i) introducing a Cpf1 polypeptide or a nucleic acid encoding a Cpf1 polypeptide into the cell; and ii) introducing a single heterologous nucleic acid comprising two or more pre-CRISPR RNAs (pre-crRNAs) either as RNA or encoded as DNA and under the control of one promoter into the cell, each pre-crRNA comprising a repeat-spacer array or repeat-spacer, wherein the spacer comprises a nucleic acid sequence that is complementary to a target sequence in the DNA and the repeat comprises a stem-loop structure, wherein the Cpf1 polypeptide cleaves the two or more pre-crRNAs upstream of the stem-loop structure to generate two or more intermediate crRNAs, wherein the two or more intermediate crRNAs are processed into two or more mature crRNAs, and wherein each two or more mature crRNAs guides the Cpf1 polypeptide to effect two or more double-strand breaks (DSBs) into the DNA. For example, one advantage of Cpf1 is that it is possible to introduce only one pre-crRNA which comprises several repeat-spacer units, which upon introduction, is processed by Cpf1 it into active repeat-spacer units targeting several different sequences on the DNA.

In another aspect, provided herein is a method for editing or modifying DNA at multiple locations in a cell consisting essentially of: i) introducing a form of Cpf1 with reduced endoribonuclease activity, as a polypeptide or a nucleic acid encoding a Cpf1 polypeptide into the cell; and ii) introducing a single heterologous nucleic acid comprising two or more pre-CRISPR RNAs (pre-crRNAs), intermediate crRNAs or mature crRNAs either as RNA or encoded as DNA and under the control of one or more promoters, each crRNA comprising a repeat-spacer array, wherein the spacer comprises a nucleic acid sequence that is complementary to a target sequence in the DNA and the repeat comprises a stem-loop structure, wherein the Cpf1 polypeptide binds to one or more regions of the single heterologous RNA with reduced or absent endoribonuclease activity and with intact endonuclease activity as directed by one or more spacer sequences in the single heterologous nucleic acid.

In some embodiments the pre-crRNA sequences in the single heterologous nucleic acid are joined together in specific locations, orientations, sequences or with specific chemical linkages to direct or differentially modulate the endonuclease activity of Cpf1 at each of the sites specified by the different crRNA sequences.

In another aspect, provided herein is an example of a general method for editing or modifying the structure or function of DNA at multiple locations in a cell consisting essentially of: i) introducing an RNA-guided endonuclease, such as Cpf1, as a polypeptide or a nucleic acid encoding the RNA-guided endonuclease into the cell; and ii) introducing a single heterologous nucleic acid comprising or encoding two or more guide RNAs, either as RNA or encoded as DNA and under the control of one or more promoters, wherein the activity or function of the RNA-guided endonuclease is directed by the guide RNA sequences in the single heterologous nucleic acid.

In some embodiments of the method, the nucleic acid encoding the Cpf1 polypeptide is a modified nucleic acid, for example, codon optimized.

In some embodiments of the method, the single heterologous nucleic acid is a modified nucleic acid.

In some embodiments of the method, the method further comprises introducing into the cell a polynucleotide donor template. In some embodiments, the polynucleotide donor template is physically linked to a crRNA or guide RNA.

In some embodiments of the method, the DNA is repaired at DSBs by either homology directed repair, non-homologous end joining, or microhomology-mediated end joining.

In some embodiments of the method, the DNA is corrected at each of the two or more DSBs by either deletion, insertion, or replacement of the DNA.

In yet another aspect, provided herein is a composition for editing a gene at multiple locations in a cell consisting essentially of: i) a Cpf1 polypeptide or a nucleic acid encoding a Cpf1 polypeptide; and ii) a single heterologous nucleic acid comprising two or more pre-CRISPR RNAs (pre-crRNAs) as RNA or encoded as DNA under the control of one promoter into the cell, each pre-crRNA comprising a repeat-spacer array, wherein the spacer comprises a nucleic acid sequence that is complementary to a target sequence in the DNA and the repeat comprises a stem-loop structure.

In some embodiments of the composition, the nucleic acid encoding the Cpf1 polypeptide is a modified nucleic acid, for example, codon optimized.

In some embodiments of the composition, the single heterologous nucleic acid is a modified nucleic acid.

In some embodiments, the composition further comprises a polynucleotide donor template. In some embodiments, the polynucleotide donor template is physically linked to a crRNA or guide RNA.

In another aspect, provided herein is a method for processing pre-crRNA into crRNA by a Cpf1 polypeptide in a manner that renders the mature crRNA available in the appropriate local milieu for directing the Cpf1 DNA endonuclease activity.

In some embodiments of the method, the Cpf1 polypeptide is more readily complexed with a mature crRNA in the local milieu, and thus more readily available for directing DNA endonuclease activity as a consequence of the crRNA being processed by the same Cpf1 polypeptide from the pre-crRNA in the local milieu.

In some embodiments of the method, the Cpf1 polypeptide is used to cleave, isolate or purify one or more mature crRNA sequences from a modified pre-crRNA oligonucleotide sequence in which heterologous sequences are incorporated 5′ or 3′ to one or more crRNA sequences within RNA oligonucleotide or DNA expression construct. The heterologous sequences can be incorporated to modify the stability, half life, expression level or timing, interaction with the Cpf1 polypeptide or target DNA sequence, or any other physical or biochemical characteristics known in the art.

In some embodiments of the method, the pre-crRNA sequence is modified to provide for differential regulation of two or more mature crRNA sequences within the pre-crRNA sequence, to differentially modify the stability, half life, expression level or timing, interaction with the Cpf1 polypeptide or target DNA sequence, or any other physical or biochemical characteristics known in the art.

In some embodiments, the Cpf1 polypeptide (or nucleic acid encoded variants thereof) is modified to improve desired its characteristics such as function, activity, kinetics, half life or the like. One such non-limiting example of such a modification is to replace a ‘ cleavage domain’ of Cpf1 with a homologous or heterologous cleavage domain from a different nuclease, such as the RuvC domain from the Type II CRISPR-associated nuclease Cas9.

In one aspect, provided herein is a method for targeting, editing or manipulating DNA in a cell comprising linking an intact or partially or fully deficient Cpf1 polypeptide or pre-crRNA or crRNA moiety, to a dimeric FOK1 nuclease to direct endonuclease cleavage, as directed to one or more specific DNA target sites by one or more crRNA molecules. In another embodiment, the FOK1 nuclease system is a nickase or temperature sensitive mutant or any other variant known in the art.

In some embodiments, the Cpf1 polypeptide linked with a dimeric FOK1 nuclease is introduced into the cell together with a single heterologous nucleic acid comprising two or more pre-CRISPR RNAs (pre-crRNAs) either as RNA or encoded as DNA and under the control of one promoter into the cell, each pre-crRNA comprising a repeat-spacer array, wherein the spacer comprises a nucleic acid sequence that is complementary to a target sequence in the DNA and the repeat comprises a stem-loop structure, wherein the Cpf1 polypeptide cleaves the two or more pre-crRNAs upstream of the stem-loop structure to generate two or more intermediate crRNAs.

In one aspect, provided herein is a method for targeting, editing or manipulating DNA in a cell comprising linking an intact or partially or fully deficient Cpf1 polypeptide or pre-crRNA, intermediate crRNA, mature crRNA moiety, or gRNA (collectively referred to as crRNA), to a donor single or double strand DNA donor template to facilitate homologous recombination of exogenous DNA sequences, as directed to one or more specific DNA target sites by one or more guide RNA or crRNA molecules.

In yet another aspect, provided herein is a method for directing a DNA template, for homologous recombination or homology-directed repair, to the specific site of gene editing. In this regard, a single stranded or double stranded DNA template is linked chemically or by other means known in the art to a crRNA or guide RNA. In some embodiments the DNA template remains linked to the crRNA or guide RNA; in yet other examples, Cpf1 cleaves the crRNA or guide RNA, liberating the DNA template to enable or facilitate homologous recombination.

In yet another aspect, provided herein is a method for targeting, editing or manipulating DNA in a cell comprising linking an intact or partially or fully deficient Cpf1 polypeptide or pre-crRNA or crRNA moiety, to a transcriptional activator or repressor, or epigenetic modifier such as a methylase, demethylase, acetylase, or deacetylase, or signaling or detection, all aspects of which have been previously described for Cas9 endonuclease systems, as directed to one or more specific DNA target sites by one or more crRNA molecules.

In another aspect, provided herein is a composition comprising a polynucleotide donor template linked to a crRNA or a guide RNA.

A method for targeting, editing or manipulating DNA in a cell comprising linking a pre-crRNA or crRNA or guide RNA to a donor single or double strand polynucleotide donor template such that the donor template is cleaved from the pre-crRNA or crRNA or guide RNA by a Cpf1 polypeptide, thus facilitating homology directed repair by the donor template, as directed to one or more specific DNA target sites by one or more guide RNA or crRNA molecules.

In yet another aspect, provided herein is a method for targeting or manipulating RNA in a cell comprising linking a Cpf1 polypeptide deficient in endoribonuclease activity to functional protein components for detection, inter-molecular interaction, translational activation, modification, or any other manipulation known in the art.

In some embodiments, the Cpf1 is selected from the group consisting of: F. novicida U112, Prevotella albensis, Acidaminococcus sp. BV3L6, Eubacterium eligens CAG:72, Butyrivibrio fibrisolvens, Smithella sp. SCADC, Flavobacterium sp. 316, Porphyromonas crevioricanis and Bacteroidetes oral taxon 274.

### BRIEF DESCRIPTION OF THE DRAWINGS

FIGS. 1A-1C show a multiple sequence alignment of Cpf1 amino acid sequences of F. novicida U112 (Fno) (gi: 118496615), Prevotella albensis M384 (Pal) (gi: 640557447), Acidaminococcus sp. BV3L6 (Asp) (gi: 545612232), Eubacterium eligens CAG:72 (Eel) (gi|547479789), Butyrivibrio fibrisolvens (Bfi) (gi: 652963004), Smithella sp. SCADC (Ssp) (gi: 739526085), Flavobacterium sp. 316 (Fsp) (gi: 800943167), Porphyromonas crevioricanis (Pcr) (gi: 739008549) and Bacteroidetes oral taxon 274 (Bor) (gi: 496509559) done with MUSCLE. Only the C-terminal region corresponding to amino acid residues 800 to 1300 of F. novicida Cpf1 is visualised by JalView. Conserved residues are shown in bold. Residues involved in RNA processing (H843, K852, K869, F873) and DNA targeting (D917, E920, H922, Y925, E1006, Y1024, E1028, D1227, D1255) are indicated by an asterisk. FIG. 1A show first part of the alignment. FIG. 1B shows the second part of the alignment. FIG. 1C show the third part of the alignment. The alignment is between residues 800-1300 of Fno (SEQ ID NO:2), 744-1253 of Pal (SEQ ID NO:3), 757-1307 Asp (SEQ ID NO:4), 722-1282 Eel (SEQ ID NO:5), 714-1231 of Bfi (SEQ ID NO:6), 745-1250 of Ssp (SEQ ID NO:7), 769-1273 of Fsp (SEQ ID NO:9), 761-1260 of Pcr (SEQ ID NO:9), and 748-1262 of Bor (SEQ ID NO:10).

FIG. 2A shows that Cpf1 processes pre-crRNA upstream of the repeat stem-loop structure. In FIG. 2A, a 5′ end labeled 69-nt long transcript consisting of a short form of pre-crRNA (repeat-spacer, full-length) was subjected to alkaline hydrolysis generating a single nucleotide resolution ladder (OH) (Ambion), and to RNase T1 (Ambion) specific cleavage to allow size determination of RNA fragments (T1). Incubation of Cpf1 (1 μM) in the presence of 10 mM MgCl₂ with internally labeled 69-nt pre-crRNA (200 nM) at 37° C. over a time course of 10 min reveals Cpf1 processing within the repeat, 4 nt upstream of the stem loop structure, yielding a 19-nt repeat fragment and a 50-nt repeat-spacer crRNA fragment. FIG. 2B is a schematic representation of a pre-crRNA repeat structure (modeled using RNAfold29 and VARNA 30). The Cpf1 cleavage site is indicated by a triangle.

FIGS. 3A-3E show that Cpf1 cleaves target DNA specifically at the 5′-YTN-3′ PAM distal end to generate 5 nt 5′-overhangs in presence of Ca²⁺. FIG. 3A shows the results of plasmid cleavage assays. Cpf1 programmed with crRNA (repeat-spacer, processed) containing spacer 4 or 5 (crRNA-sp4 or crRNA-sp5) was used to target a supercoiled plasmid DNA comprising protospacer 5 in absence or presence of Ca²⁺. FIG. 3B shows the results of oligonucleotide cleavage assays. Cpf1 programmed with crRNA-sp4 or crRNA-sp5 was used to target an oligonucleotide duplex in the absence or presence of Ca²⁺. The target or non-target strand was 5′ radiolabeled prior to annealing to the non-labeled complementary strand to form the substrate duplex. FIG. 3C shows a schematic representation of the oligonucleotide duplex used in FIG. 3B, and the structure of crRNA-sp5 used in FIG. 3A and FIG. 3B. Cleavage sites corresponding to fragments obtained in FIG. 3B and confirmed by sequencing (FIG. 13) are indicated by triangles. The PAM sequence is marked by a box. FIG. 3D shows the Cpf1 PAM determination. Plasmid DNA containing protospacer 5 and the PAMs 1-6, or 5′ radiolabeled ds oligonucleotide containing protospacer 5 and PAMs 1 and 7-9 were subjected to cleavage by Cpf1 programmed with crRNA-sp5 in the presence of 10 mM CaCl₂ (upper and lower panel, respectively). FIG. 3E shows results of the seed sequence determination experiments. Plasmids containing protospacer 5 and single or quadruple mismatches along the target strand were tested for cleavage by Cpf1 programmed with crRNA-sp5 in the presence of 10 mM MgCl₂. Labeled: li, linear; sc, super coiled; M, 1 kb ladder (Fermentas). Sizes of oligonucleotide cleavage products are indicated in nucleotides. Quantification of FIG. 3E is shown below in the table.

| substrate | wt | T22G | C21A | T20G | A19C | A18C | T17G | T16G | T15G |
|---|---|---|---|---|---|---|---|---|---|
| % cleavage | 83 ± 15 | 37 ± 1 | 41 ± 2 | 22 ± 3 | 30 ± 2 | 33 ± 4 | 28 ± 11 | 39 ± 18 | 57 ± 2 |
| substrate | T14G | C13A | C12A | A11C | T10G | T9G | A8C | A7C | G6T |
| % cleavage | 69 ± 9 | 77 ± 13 | 87 ± 6 | 68 ± 12 | 79 ± 5 | 100 ± 0 | 65 ± 25 | 79 ± 16 | 92 ± 14 |
| substrate | A5C | T4G | A3C | G2T | A1C | Mut_1-4 | | Mut_19-22 | |
| % cleavage | 75 ± 35 | 55 ± 27 | 62 ± 19 | 66 ± 24 | 64 ± 24 | 47 ± 25 | | 0 | |

Percent cleavage is the result of three independent experiments ± standard deviation.

FIGS. 4A-4D show that Cpf1 contains two active centers for RNA and DNA cleavage. In FIG. 4A, Cpf1_wt, Cpf1_H843A, Cpf1_K852A, Cpf1_K869A and Cpf1_F873A were tested for DNA cleavage activity (upper panel), in vitro RNA cleavage activity (middle panel) and in vivo RNA processing activity (lower panel). DNA cleavage was performed on a protospacer 5 containing plasmid with crRNA-sp5 (repeat-spacer, full-length) in the presence of 10 mM MgCl₂. In vitro RNA cleavage was performed on internally labeled pre-crRNA (repeat-spacer, full-length) in the presence of 10 mM MgCl2. In vivo RNA processing was analyzed by Northern Blot, probing against the spacer of a pre-crRNA (repeat-spacer-repeat, full-length). In FIG. 4B, Cpf1_wt, Cpf1_D917A, Cpf1_E1006A and Cpf1_D1255A were tested for DNA cleavage activity (upper panel) and in vitro RNA cleavage activity (lower panel). Assays were performed as described in FIG. 4A. FIG. 4C shows DNA cleavage activity of Cpf1_E920A, Cpf1_H922A, Cpf1_Y925A, Cpf1_Y1024A, Cpf1_E1028A and Cpf1_D1227A on ds oligonucleotide substrates containing protospacer 5. Target or non-target strand was 5′ radiolabeled prior to annealing to the non-labeled complementary strand to form an oligonucleotide duplex. The cleavage reactions were done in the presence of 10 mM CaCl₂ (upper two panels) or MgCl₂ (lower two panels). FIG. 4D is a schematic representation of the Cpf1 amino-acid sequence with the active domains for RNA and DNA cleavage are shaded. The mutated amino acids are indicated; mutated amino acids are indicated with the DNase motif shown in bold font. Labeled: li, linear; sc, supercoiled. The sizes of RNA or oligonucleotide cleavage products and Northern blot fragments are indicated in nucleotides.

FIGS. 5A-5B show that F. novicida U112 expresses short mature Type V-A crRNAs composed of repeat-spacer. FIG. 5A shows an in-scale representation of Type II-B (cas9) and Type V-A (cpf1) CRISPR-Cas loci in F. novicida U112. Cas genes; putative pre-crRNA promoters; CRISPR leader sequence; CRISPR repeats; CRISPR spacers; tracrRNA or scaRNA are shown as various elements. In FIG. 5B, expression of Type V-A crRNAs determined by small RNA sequencing is represented with a grey bar chart. The coverage of the reads is indicated in brackets and reads starting (5′ end) and ending (3′ end) at each position are shown (image captured from Integrative Genomics Viewer, IGV). The genomic coordinates and size of the CRISPR array in base pairs are indicated. The sequence of the Type V-A CRISPR array from the leader sequence to the last repeat is shown. Black bold uppercase sequences are repeats followed by italicized lower case sequences, spacers. The boxed sequences correspond to the mature crRNAs detected by small RNA sequencing. The mature crRNAs are composed of part of the repeat in 5′ and part of the spacer in 3′.

FIGS. 6A-6D show that wild type Cpf1 purifies as a monomer in solution. Recombinant Cpf1 of F. novicida U112 purified via affinity and cation-exchange chromatography (HiTrap Heparin, GE-Healthcare) was applied to a Superdex 200 size-exclusion column (GE-Healthcare). In FIG. 6A, protein samples obtained by size-exclusion chromatography were separated by SDS-PAGE (8% polyacrylamide) and visualised with coomassie staining. FIG. 6B shows the elution profile of the size-exclusion chromatography of wild type Cpf1. FIG. 6C shows the calibration curve of proteins with known molecular weights (Molecular Weight Marker Kit, Sigma-Aldrich). A comparative analysis of the elution volume of the peak (FIG. 6B) with the calibration curve (FIG. 6C) reveals a size of 187 kDa, indicating a monomeric form of Cpf1 in solution. FIG. 6D shows an SDS-PAGE of protein eluates obtained by metal ion-affinity purification (left panel) and cation exchange chromatography (right panel).

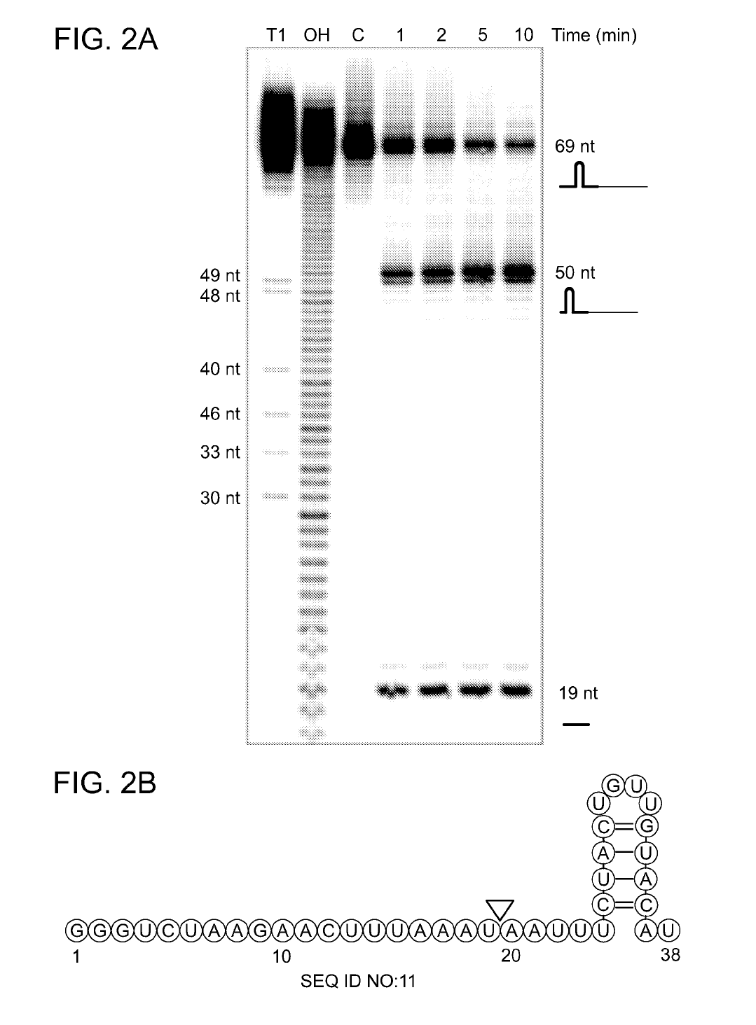

FIGS. 7A-7B show that the endoribonucleolytic activity of Cpf1 is dependent on the presence of an intact repeat sequence. FIG. 7A shows results of cleavage assays were done by incubating 100 nM of internally labeled RNA constructs corresponding to different repeat and spacer sequence variants with 1 μM of Cpf1 for 30 min at 37° C. The cleavage reaction was analysed by denaturing polyacrylamide gel electrophoresis and phosphorimaging. The cleavage products are represented schematically and the sizes are indicated in nucleotides. The sequence compositions of the RNAs used as substrates are shown in FIG. 7B. RNA structures were generated with RNAfold and visualised using VARNA software. Cpf1 cleaved only the RNA templates containing a full-length repeat sequence. The substrate containing two repeats was cleaved twice resulting in more than two fragments, while cleavage of RNAs with only one repeat resulted in two fragments, consistent with the determined cleavage site (see FIG. 2).

FIG. 8 shows that Cpf1 processes pre-crRNA in vivo. Northern Blot analysis of total RNA extracted from E. coli co-transformed with a plasmid encoding pre-crRNA and either the empty vector or overexpression vectors encoding Cpf1 wild type and variants. Cpf1 expression was induced (+) or not induced (−) with IPTG. The Northern Blot was probed against the spacer sequence of the tested pre-crRNA. In absence of Cpf1 (empty vector or not induced), the amount of transcript was reduced compared to in presence of Cpf1, indicating a stabilisation of pre-crRNA by binding of Cpf1. Expression of Cpf1 resulted in a distinct processed transcript, while expression of Cpf1_H843A, Cpf1_K852A and Cpf1_K869A resulted in several higher transcripts. Expression of Cpf1_F873A resulted in almost undetectable processed transcript.

FIGS. 9A-9C shows that Cpf1 is a sequence- and structure-specific endoribonuclease. Design of various repeat variants of pre-crRNA-sp5 (pre-crRNA with spacer 5) with an altered repeat sequence, a destroyed repeat structure, single nucleotide exchanges (1-4) in the repeat recognition sequence (RRS) and changed loop and stem sizes. Note that the 5′ repeat region of the wild-type repeat is not shown in the different variants. Darker shaded circles highlight the mutated or added residues. The RNA structures were generated with RNAfold and visualized using VARNA software. FIG. 9A was generated as follows. Internally labeled pre-crRNAs containing a wild-type repeat sequence, an altered repeat sequence or a destroyed repeat structure were obtained by in vitro transcription. The 5′ end-labeled wild-type substrate was used to generate an alkaline hydrolysis ladder (OH) and an RNase T1 digest (T1) for size determination of the RNA fragments (Life Technologies). Cpf1 cleaved only the pre-crRNA template containing the wild-type repeat sequence yielding a small 19-nt 5′ repeat fragment and a 50-nt intermediate crRNA. FIG. 9B was generated similarly, wherein substrates with serial single mutations of the four RRS nucleotides (1-4, counting from the cleavage site) were tested for processing by Cpf1. Changes of the first three nucleotides were not tolerated for Cpf1-mediated processing, whereas changing the fourth nucleotide yielded a substrate that was processed with less efficiency compared to the wild-type substrate. FIG. 9C was generated in the same manner, wherein the influence of loop variations in the repeat was tested with substrates containing +1 or −1 nucleotide in the loop. Both substrates were processed by Cpf1. Stems with +1 or −1 base pair, or +4 base pairs were used to determine length requirements of the stem. Cpf1 did not cleave any of the three substrates tested. The RNA cleavage reactions were performed by incubating 1 μM of Cpf1 with 200 nM of RNA variant at 37° C. for 5 min in the presence of 10 mM MgCl₂. The cleavage products were analyzed by denaturing polyacrylamide gel electrophoresis and phosphorimaging. RNA fragments are represented schematically and fragment sizes are indicated in nucleotides.

FIGS. 10A-10B show that the DNA and RNA cleavage activities of Cpf1 are dependent on divalent metal ions. FIG. 10A shows RNA cleavage assays of pre-crRNA-sp5 with Cpf1 in KGB supplemented with different concentrations of divalent metal ion (indicated in mM) or EDTA (10 mM). Cleavage products were analysed by denaturing polyacrylamide gel electrophoresis and visualized by phosphorimaging. RNA fragments are represented schematically and fragment sizes are indicated in nucleotides. Specific RNA cleavage was observed in the presence of MgCl₂. Less specific cleavage was detected with CaCl₂, MnCl₂ and CoCl₂. FIG. 10B shows cleavage assays of supercoiled plasmid DNA containing protospacer 5 by Cpf1 programmed with crRNA-sp5 in KGB buffer supplemented with different concentrations of divalent metal ions (indicated in mM). Cleavage products were analysed by agarose gel electrophoresis and visualized by EtBr staining. DNA cleavage was observed in the presence of MgCl₂ and MnCl₂. A more specific cleavage was observed in the presence of CaCl₂. li, linear; sc, supercoiled; M, 1 kb ladder (Fermentas). Quantification of data in FIG. 10B is known below in the table

| ion | Ca²⁺ | | Mg²⁺ | | Mn²⁺ | |
|---|---|---|---|---|---|---|
| concentration | 1 mM | 10 mM | 1 mM | 10 mM | 1 mM | 10 mM |
| % cleavage | 44 ± 17 | 82 ± 8 | 13 ± 10 | 84 ± 10 | 39 ± 17 | 86 ± 2 |
| ion | Co²⁺ | | Ni²⁺ | | Zn²⁺ | |
| concentration | 1 mM | 10 mM | 1 mM | 10 mM | 1 mM | 10 mM |
| % cleavage | 0 | 0 | 0 | 0 | 0 | 0 |

* Percent cleavage is the result of three independent experiments ± standard deviation.

Below is a summary of recognized substrates, metal ion dependency and crRNA requirements for both RNase and DNase motifs of Cpf1. − no activity; + residual activity; +++ full activity.

| | RNase | DNase |
|---|---|---|
| Substrate | | |
| RNA | +++ | − |
| DNA | − | +++ |
| Dependency | | |
| Mg²⁺ | +++ | +++ |
| Ca²⁺ | + | +++ |
| crRNA repeat sequence | +++ | + |
| crRNA repeat structure | +++ | +++ |

FIGS. 11A-11D show that Cpf1 requires crRNA with an intact repeat structure to specifically cleave DNA. FIG. 11A shows cleavage assays of supercoiled plasmid DNA containing protospacer 5 by Cpf1 programmed with different RNA constructs (1-8) in the presence of 10 mM CaCl₂. Cleavage products were analysed by agarose gel electrophoresis and visualised by EtBr staining. li, linear; sc, supercoiled; M, 1 kb ladder (Fermentas). FIG. 11B shows cleavage of 5′ radiolabeled oligonucleotide duplexes containing protospacer 5 in the presence of 10 mM CaCl₂. Cleavage products were analysed by denaturing polyacrylamide gel electrophoresis and visualised by phosphorimaging. Fragment sizes are indicated in nucleotides. The sequence compositions of the RNAs used as substrates are shown schematically in FIG. 11C and FIG. 11D. RNA structures were generated with RNAfold and visualised using VARNA software. Only the RNAs containing a full-length repeat and a spacer complementary to the target mediated DNA cleavage by Cpf1.

FIGS. 12A-12C show DNA and RNA binding studies of Cpf1. FIG. 12A shows electrophoretic mobility shift assays (EMSAs) of 5′ radiolabeled ds oligonucleotides containing protospacer 5 by Cpf1 programmed with RNA 1-6 (see FIG. 11). The protein concentrations used were 8, 52 and 512 nM. Reactions were analyzed by native PAGE and phosphorimaging. Unbound and bound DNAs are indicated. Higher DNA binding affinities are observed when Cpf1 is programmed with an RNA containing an entire repeat sequence. FIG. 12B shows EMSAs of 5′-radiolabeled double-stranded oligonucleotides containing protospacer 5 targeted by wild-type Cpf1, Cpf1 (D917A), Cpf1 (E1006A) and Cpf1 (D1255A) in complex with crRNA-sp5 (repeat-spacer 5, full length, RNA 4, FIG. 11). The protein concentrations used were 8, 16, 32, 42, 52, 64, 74, 128 and 256 nM. Reactions were analyzed by native polyacrylamide gel electrophoresis and phosphorimaging. Unbound and bound DNAs are indicated. The results shown here are representative of at least three individual experiments. The bound and unbound DNA fractions were quantified, plotted against the enzyme concentration and fitted by nonlinear regression analysis. The calculated K_{d} values (±s.d.) were 50±3 nM (wild type), 48±8 nM (D917A), 40±8 nM (E1006A) and 52±6 nM (D1255A). There are no differences between the RNA-mediated DNA binding affinities of wild-type and mutant Cpf1. The reduced K_{d} for E1 006A can be explained by the removal of the large negatively charged amino acid, which might facilitate interaction of Cpf1 with the DNA. FIG. 12C shows EMSAs of 5′-radiolabeled crRNA-sp5 (repeat-spacer 5, processed, RNA 3, FIG. 6) by wild-type Cpf1, Cpf1 (H843A), Cpf1 (K852A), Cpf1 (K869A) and Cpf1 (F873A). The protein concentrations used were 2, 4, 8, 12, 16, 24, 32, 48 and 64 nM. Reactions were analysed by native polyacrylamide gel electrophoresis and phosphorimaging. Unbound and bound RNAs are indicated. Shown are representatives of at least three individual experiments. The bound and unbound RNA fractions were quantified, plotted against the enzyme concentration and fitted by nonlinear regression analysis. The calculated Kd values (±s.d.) were 16±1 nM (wild type), 17±0.5 nM (H843A), 12±1 nM (K852A), 10±1 nM (K869A) and 17±1 nM (F873A). There are no differences between the RNA binding affinities of wild-type and mutant Cpf1.

FIGS. 13A-13D show analysis of target DNA cleavage by crRNA-programmed Cpf1 in the presence of Mg²⁺. FIG. 13A shows cleavage assays of supercoiled plasmid DNA containing protospacer 5 by Cpf1 programmed with crRNA-sp4 or crRNA-sp5 (repeat-spacer, processed) in the absence or presence of Mg²⁺. FIG. 13B shows oligonucleotide cleavage assays using Cpf1 programmed with crRNA-sp5 in the presence of Mg²⁺. Either the target or the non-target strand was 5′ radiolabeled before annealing to the non-labeled complementary strand to form the duplex substrate.

FIG. 13C shows the sequencing analysis of the cleavage product obtained in FIG. 13A. The termination of the sequencing reaction indicates the cleavage site. Note that an enhanced signal for adenine is a sequencing artefact. FIG. 13D shows the Cpf1 PAM determination. Plasmid DNA containing protospacer 5 and the PAMs 1-6, or 5′ radiolabeled ds oligonucleotide containing protospacer 5 and PAMs 1, 7-9 were subjected to cleavage by Cpf1 programmed with crRNA-sp5 (repeat-spacer, full-length) in the presence of 10 mM MgCl₂ (upper and lower panel, respectively). li, linear; sc, super coiled; M, 1 kb ladder (Fermentas). Oligonucleotide cleavage products are indicated in nucleotides.

FIG. 14 A14B demonstrate that processing activity of Cpf1 is specific for pre-crRNA and crRNA-mediated targeting of Cpf1 is directed only against single- and double-stranded DNA. In FIG. 14A, Cpf1 processing activity was tested against pre-crRNA and pre-crDNA. Wild-type Cpf1 or Cpf1(D917A) (1 μM) was incubated with 200 nM internally labeled pre-crRNA-sp5 (repeat-spacer 5, full-length, RNA 4, FIG. 11) or a 5′-labeled ssDNA (pre-crDNA-sp5) construct with the same sequence as the RNA in KGB buffer with 10 mM MgCl₂ for 5 min at 37° C. Incubation of wild-type Cpf1 and DNase inactive mutant (Cpf1 (D917A)) with the RNA construct, but not the DNA construct, resulted in the expected cleavage products of a 19-nt repeat fragment and a 50-nt intermediate crRNA, indicating that the processing activity of Cpf1 is specific for RNA. FIG. 14B shows crRNA-mediated DNA cleavage activity of Cpf1. Cpf1 (100 nM) in complex with crRNA-sp5 (repeat-spacer 5, full-length, RNA 4, 11) was incubated with 10 nM of 5′-radiolabeled ssRNA, dsRNA, ssDNA, dsDNA or RNA-DNA hybrids in KGB buffer with either MgCl₂ (10 mM; upper panel) or CaCl₂ (10 mM; lower panel) for 1 h at 37° C. The oligonucleotide DNA substrates contained the sequence for protospacer 5 targeted by the tested crRNA. For DNA-RNA hybrids, the 5′-radiolabeled target strand is indicated with an asterisk. Only ssDNA and dsDNA substrates were cleaved, indicating that the crRNA-mediated cleavage activity of Cpf1 is only directed against DNA substrates. The cleavage products for ssDNA, however, vary from those expected or observed for dsDNA. Cleavage reactions were analysed by denaturing polyacrylamide gel electrophoresis and phosphorimaging. RNA cleavage products are indicated schematically. RNA and DNA fragment sizes are given in nucleotides.

### BRIEF DESCRIPTION OF THE SEQUENCE LISTING

SEQ ID NO:1 is the coding DNA sequence (CDS) of an illustrative Cpf1 from Francisella novicida U112.

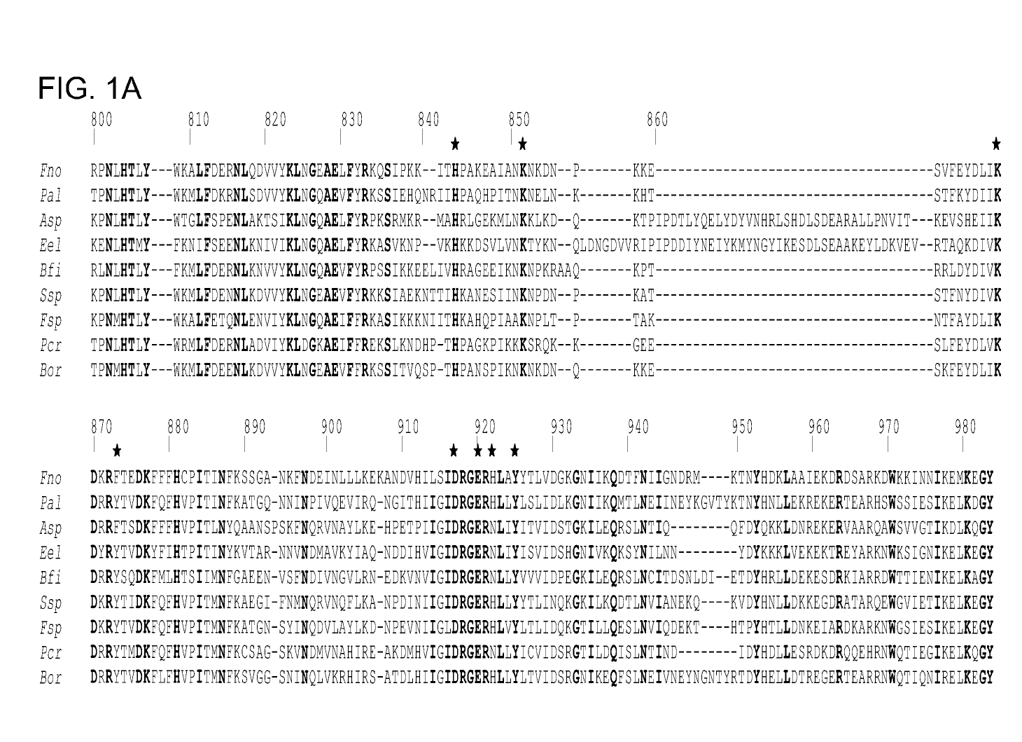

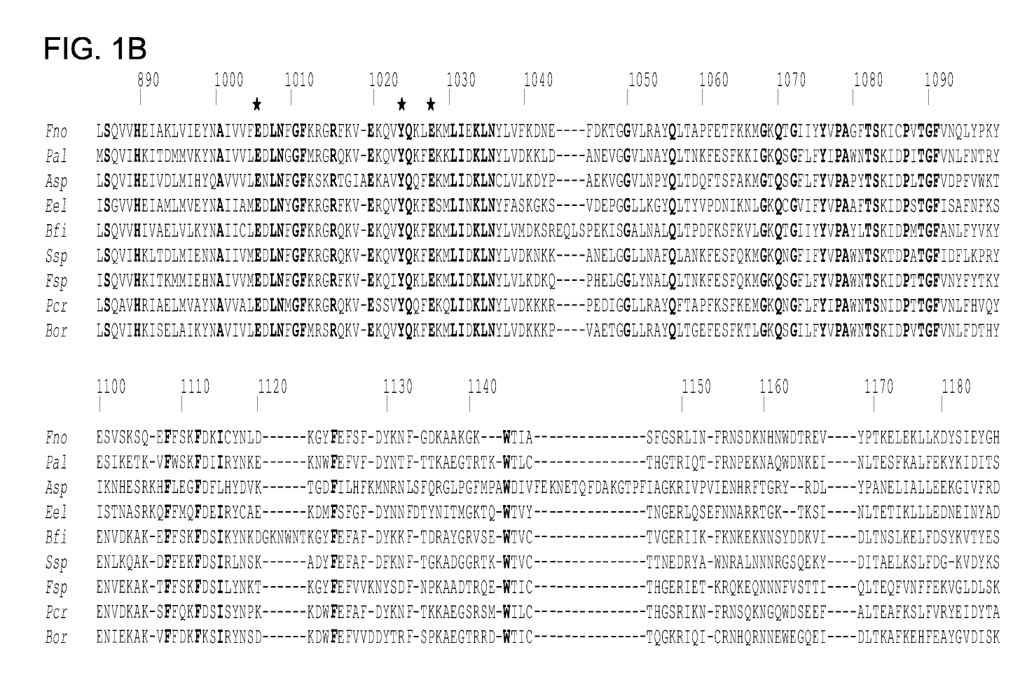

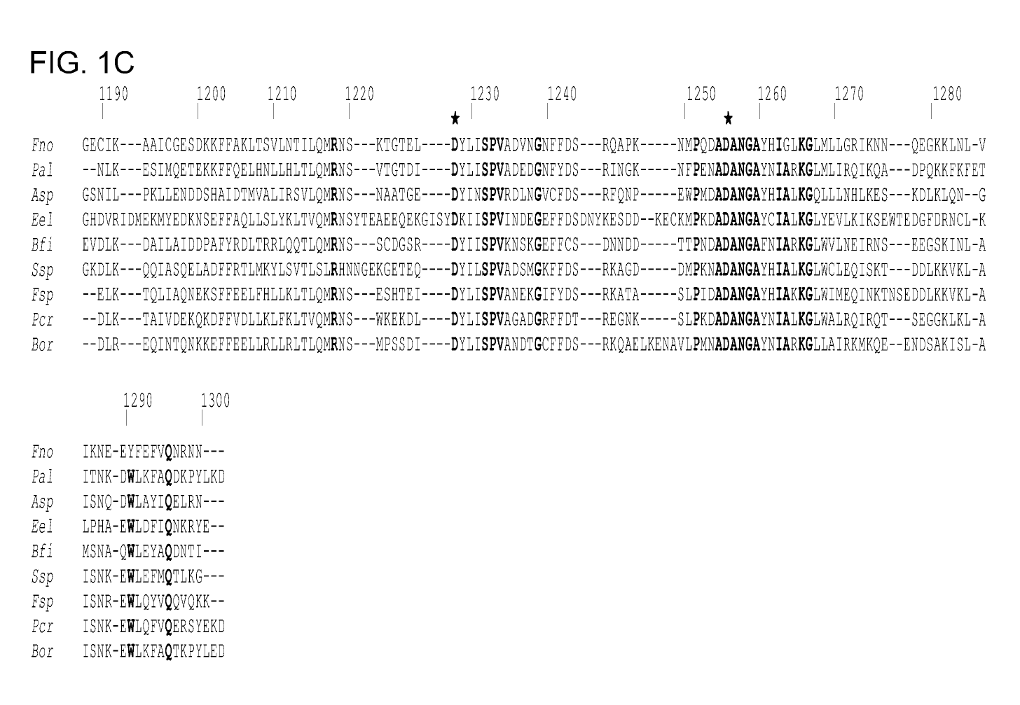

SEQ ID NO:2-10 are amino acid sequences of Cpf1 orthologues from multiple species as follows: F. novicida U112 (Fno) (gi: 118496615), Prevotella albensis M384 (Pal) (gi: 640557447), Acidaminococcus sp. BV3L6 (Asp) (gi: 545612232), Eubacterium eligens CAG:72 (Eel) (gi: 547479789), Butyrivibrio fibrisolvens (Bfi) (gi: 652963004), Smithella sp. SCADC (Ssp) (gi: 739526085), Flavobacterium sp. 316 (Fsp) (gi: 800943167), Porphyromonas crevioricanis (Pcr) (gi: 739008549) and Bacteroidetes oral taxon 274 (Bor) (gi: 496509559) done with MUSCLE. (Only the C-terminal region corresponding to amino acid residues 800-1300 of F. novicida Cpf1 is visualised in FIGS. 1A-1C. More particularly, the alignment is between residues 800-1300 of Fno (SEQ ID NO:2), 744-1253 of Pal (SEQ ID NO:3), 757-1307 Asp (SEQ ID NO:4), 722-1282 Eel (SEQ ID NO:5), 714-1231 of Bfi (SEQ ID NO:6), 745-1250 of Ssp (SEQ ID NO:7), 769-1273 of Fsp (SEQ ID NO:9), 761-1260 of Pcr (SEQ ID NO:9), and 748-1262 of Bor (SEQ ID NO:10).

SEQ ID NO:11 is an exemplary pre-crRNA repeat-spacer array structure shown in FIG. 2B.

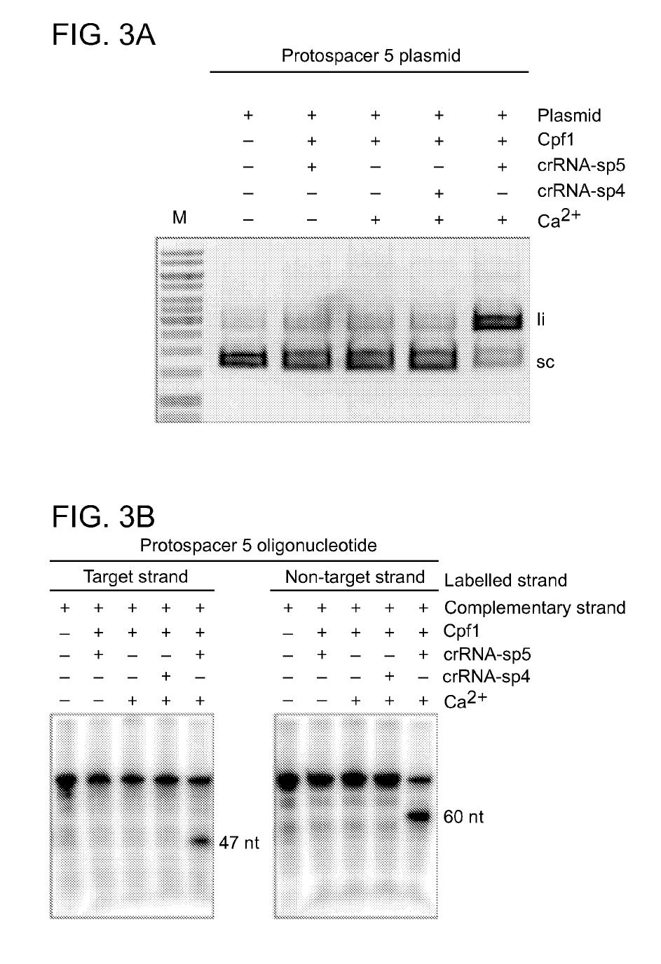

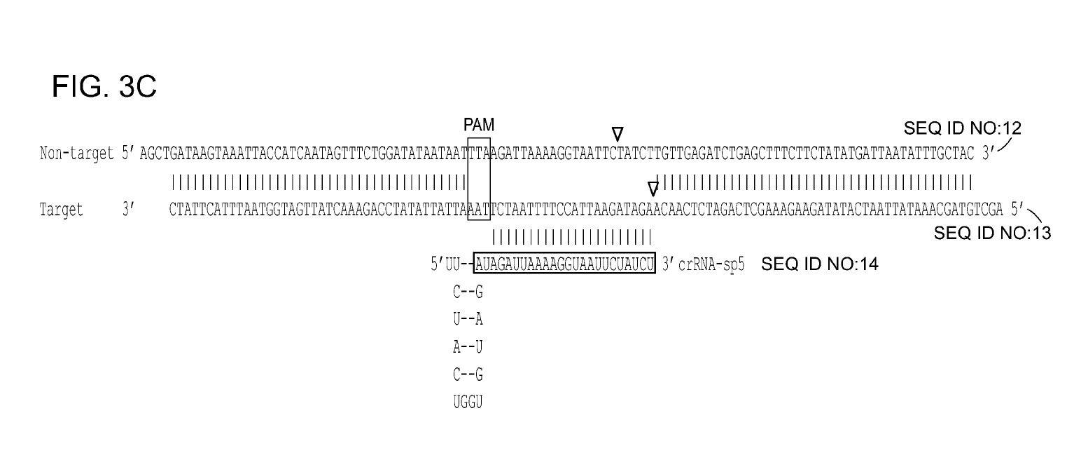

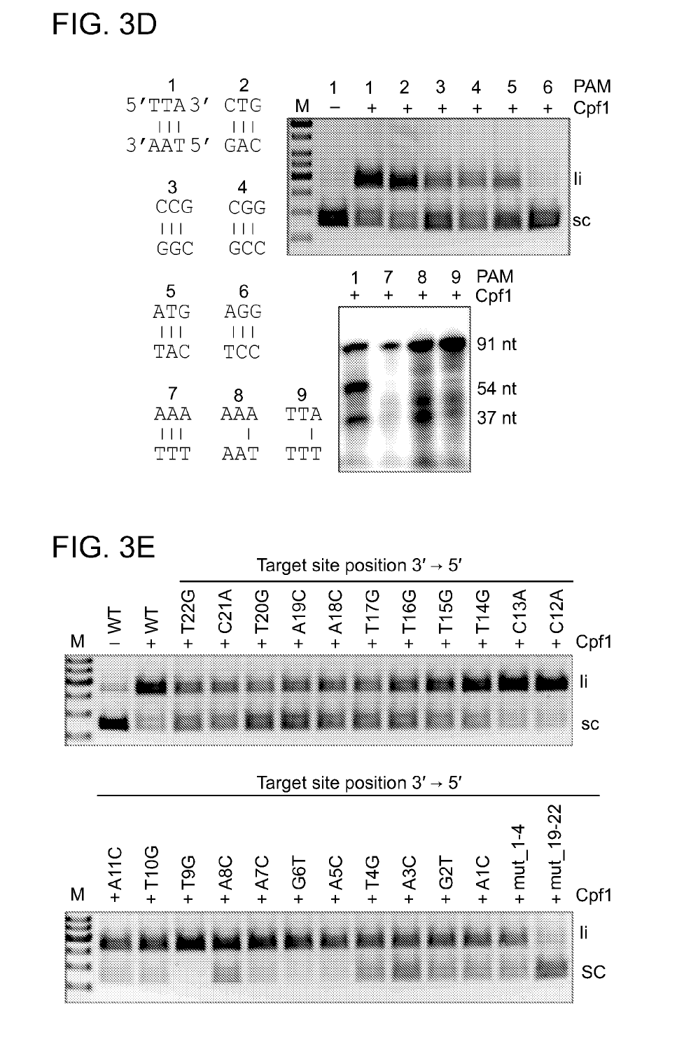

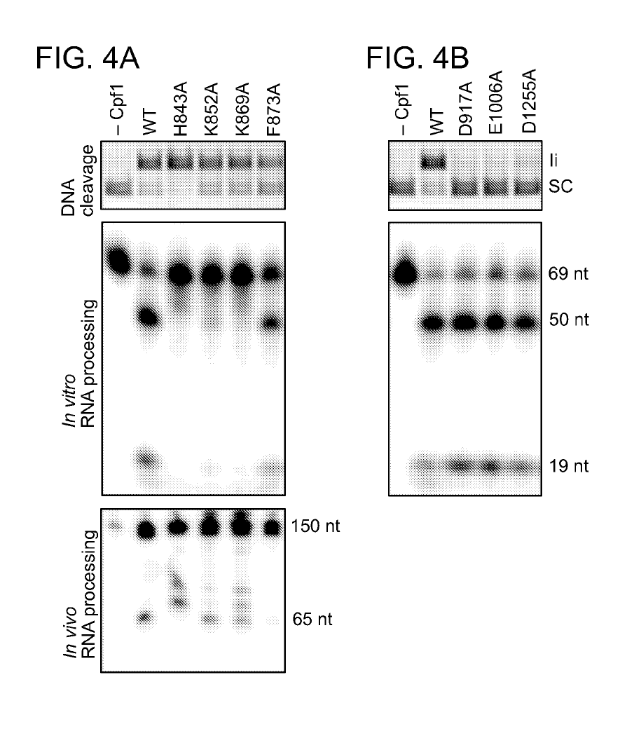

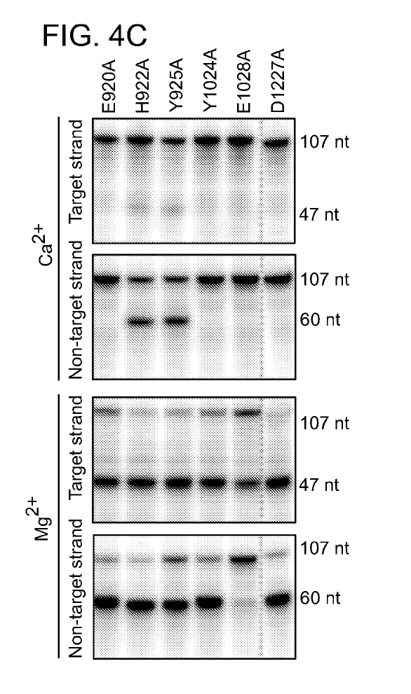

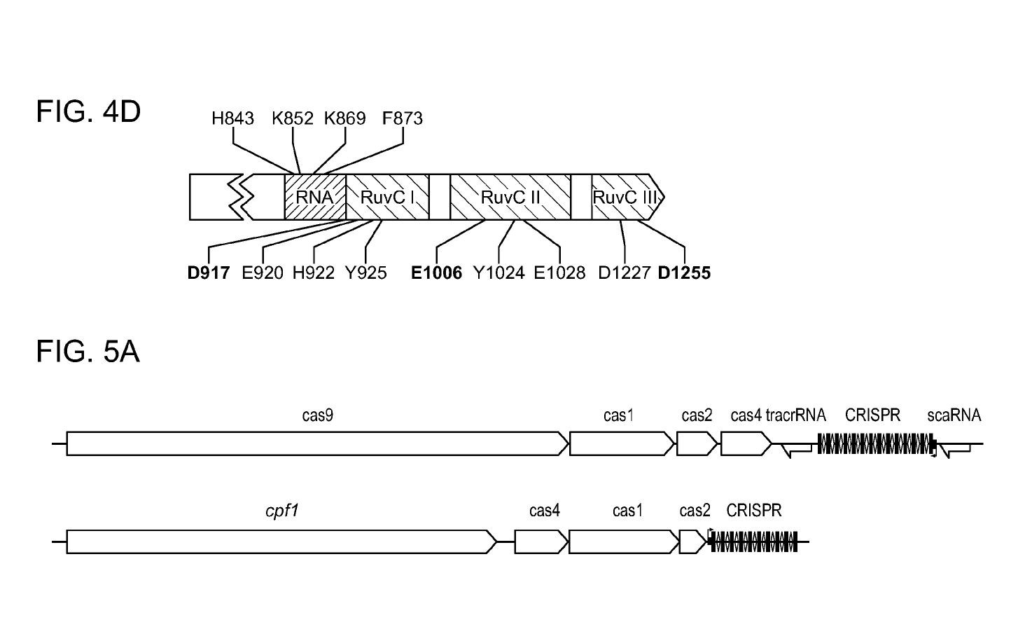

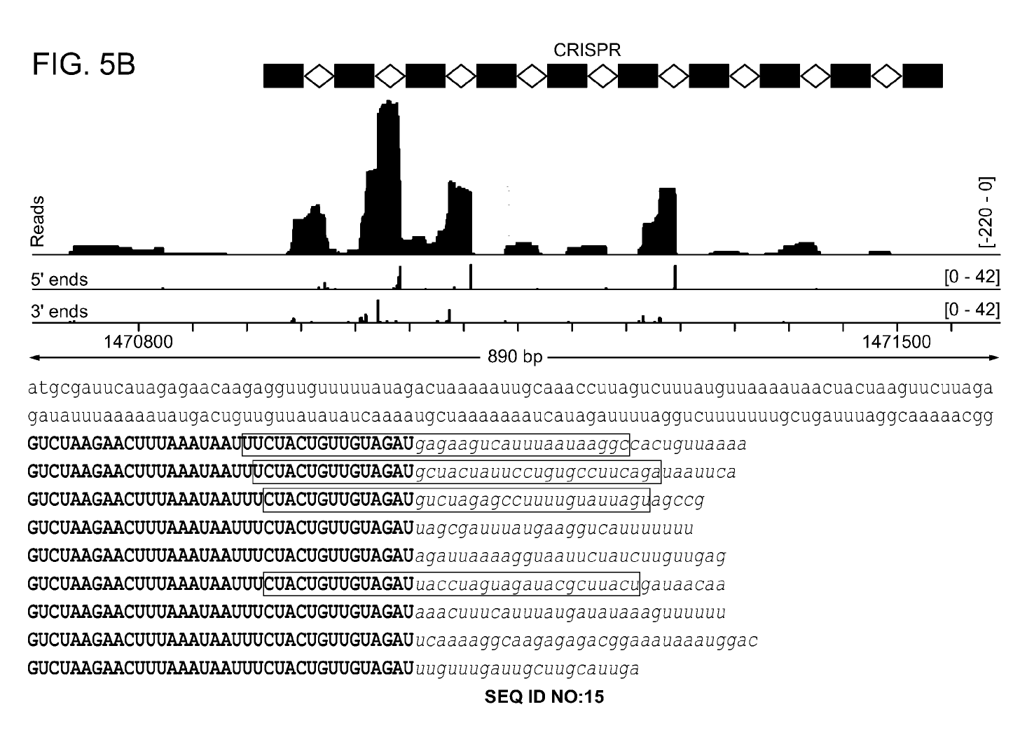

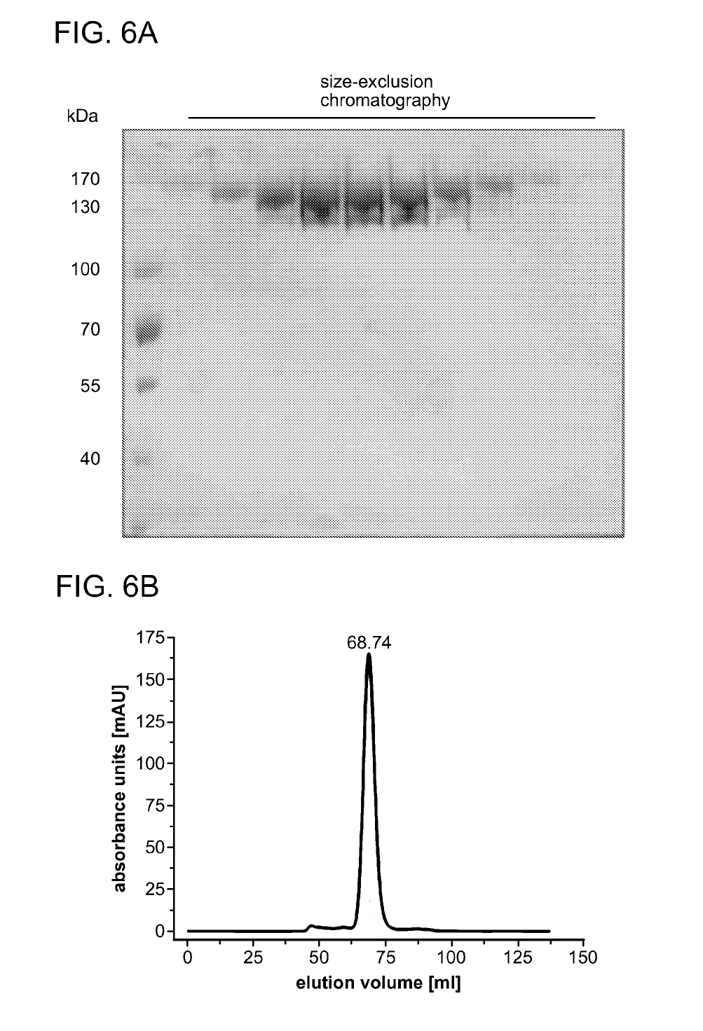

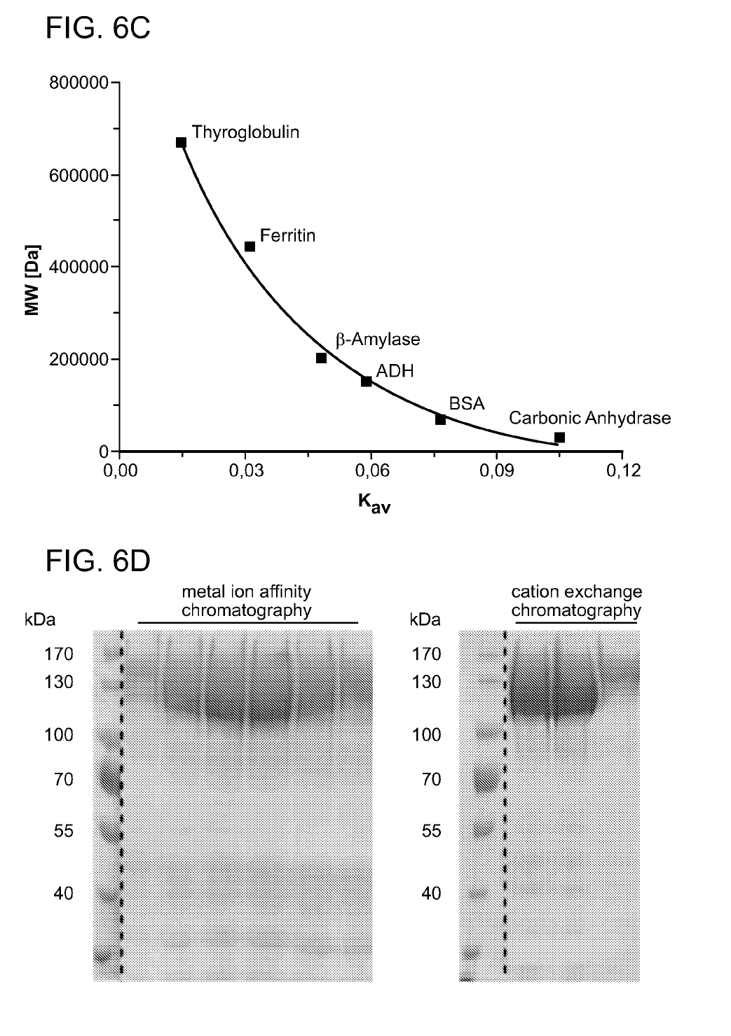

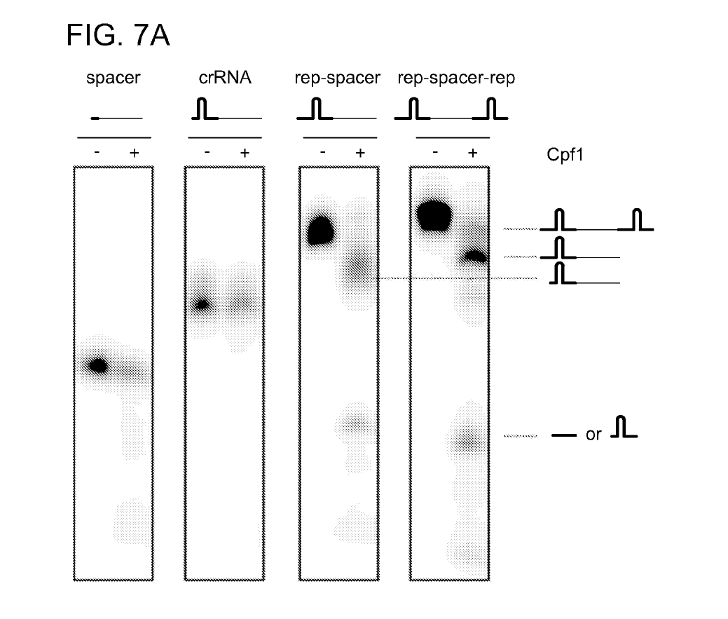

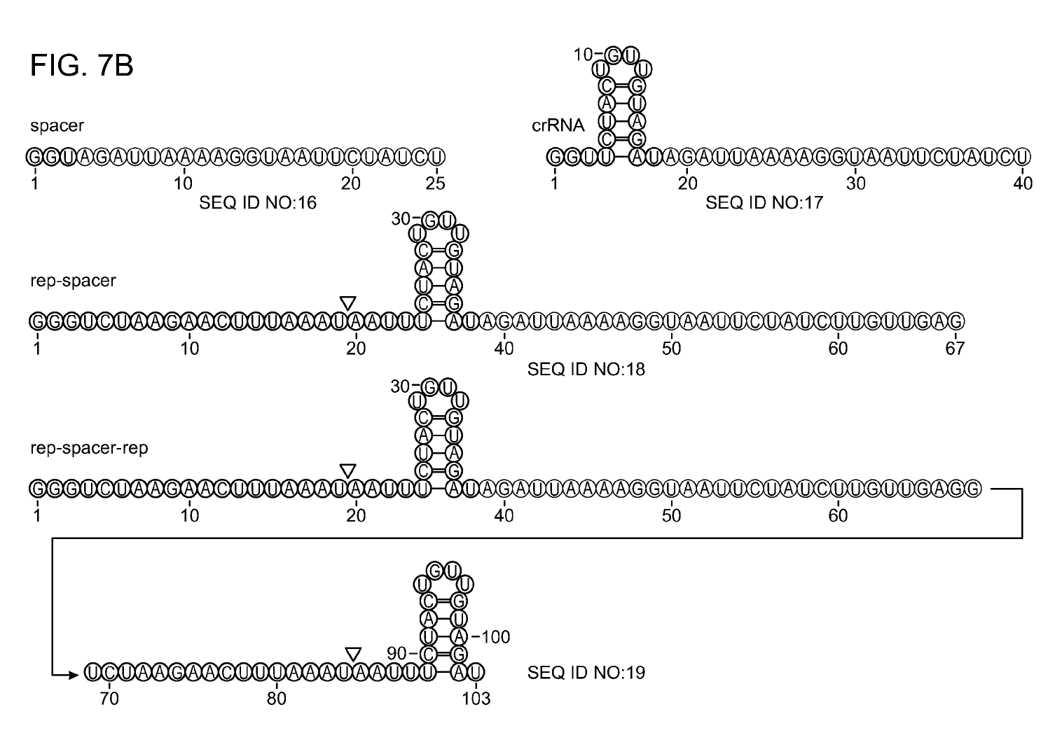

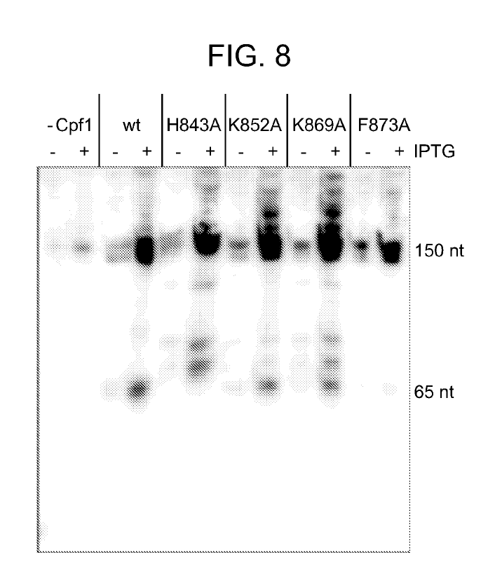

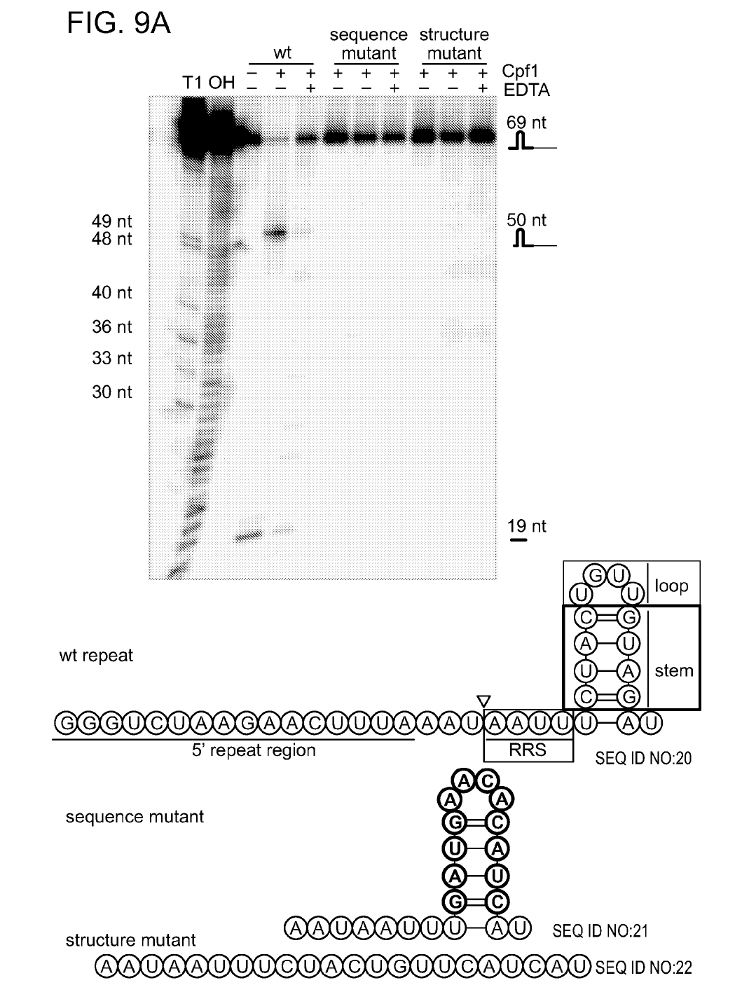

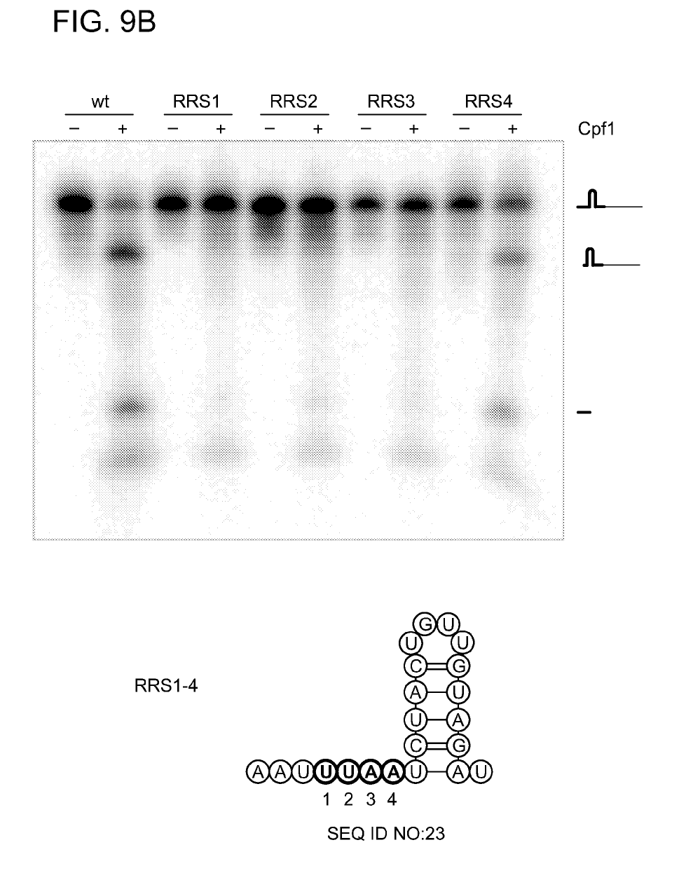

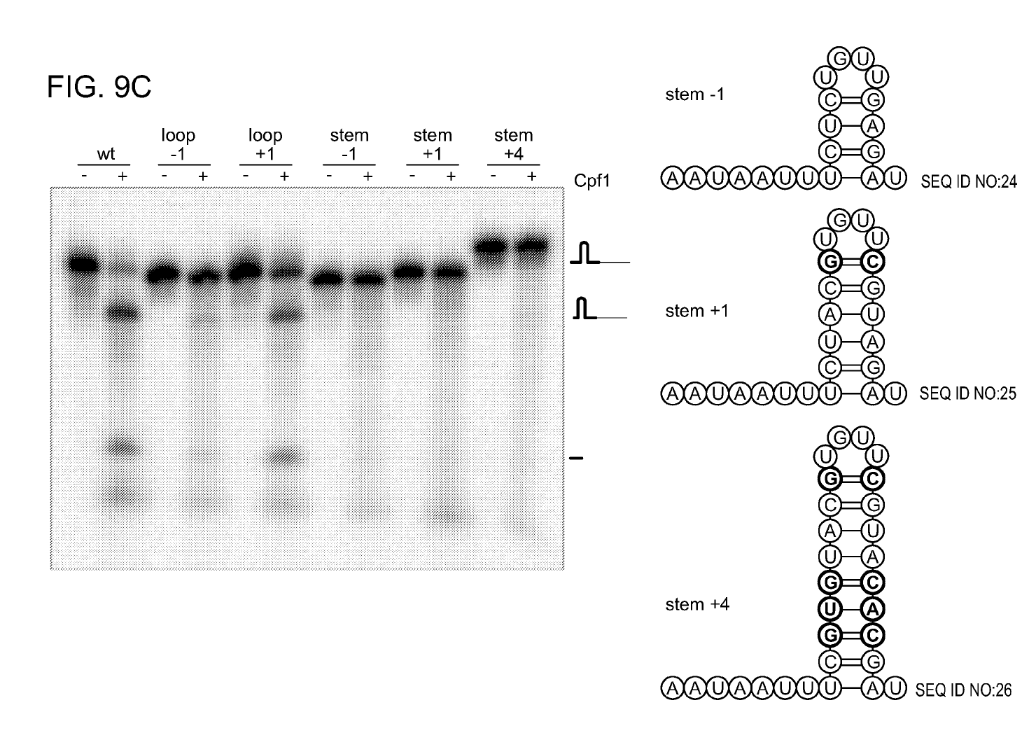

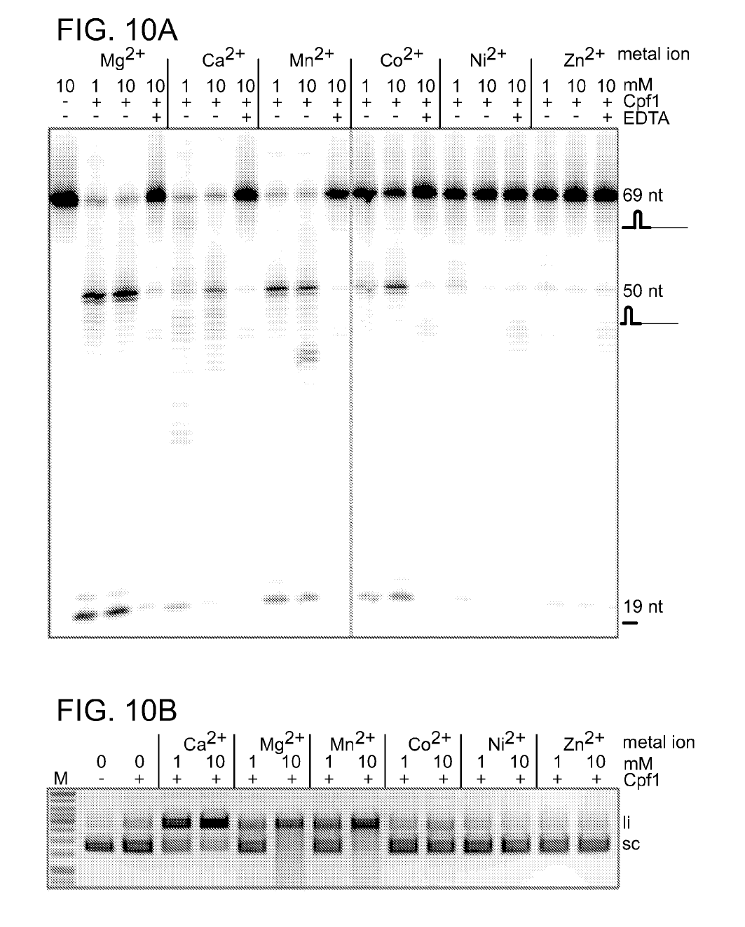

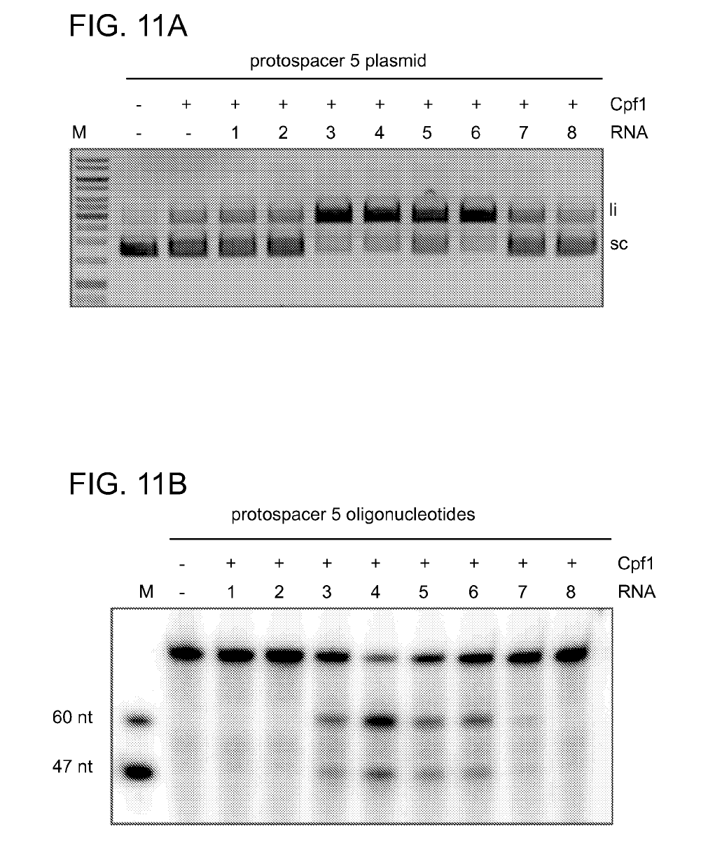

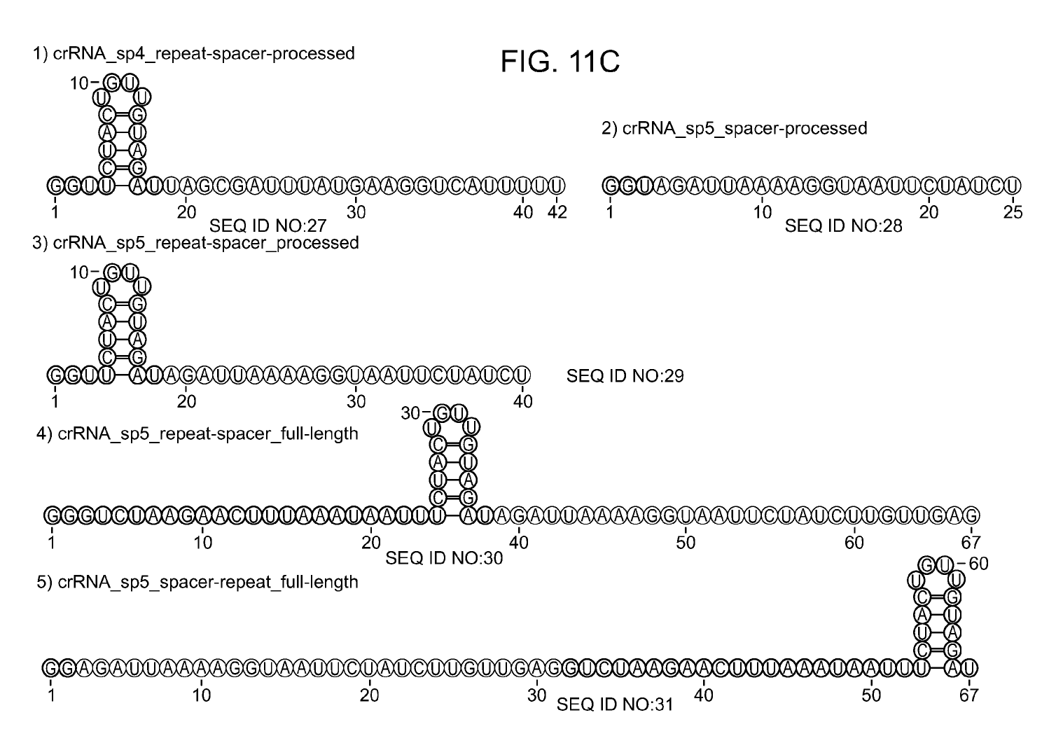

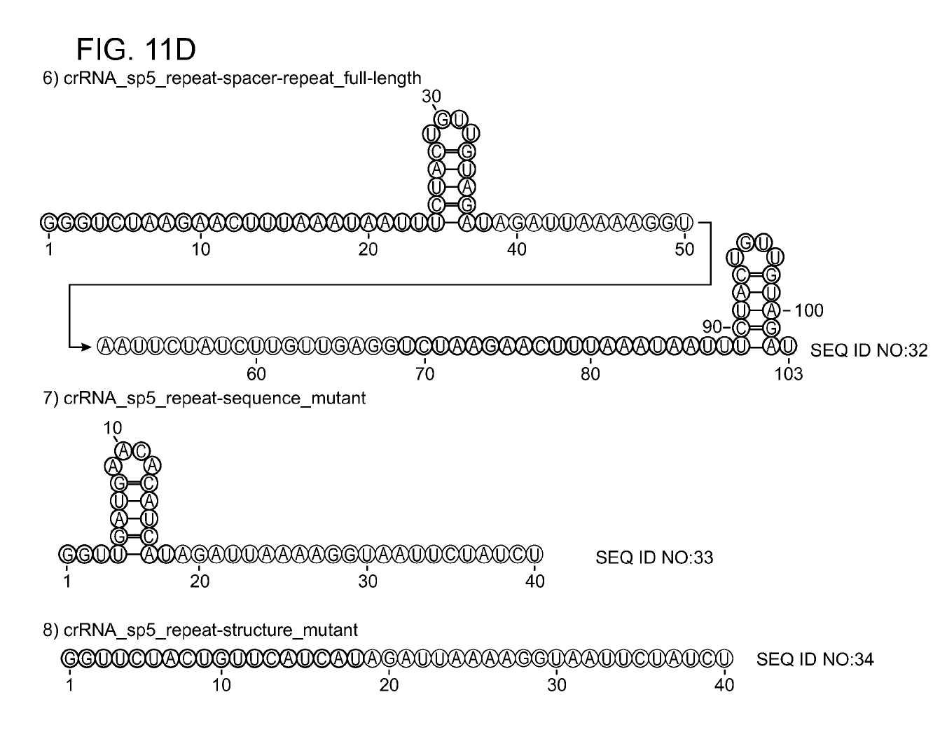

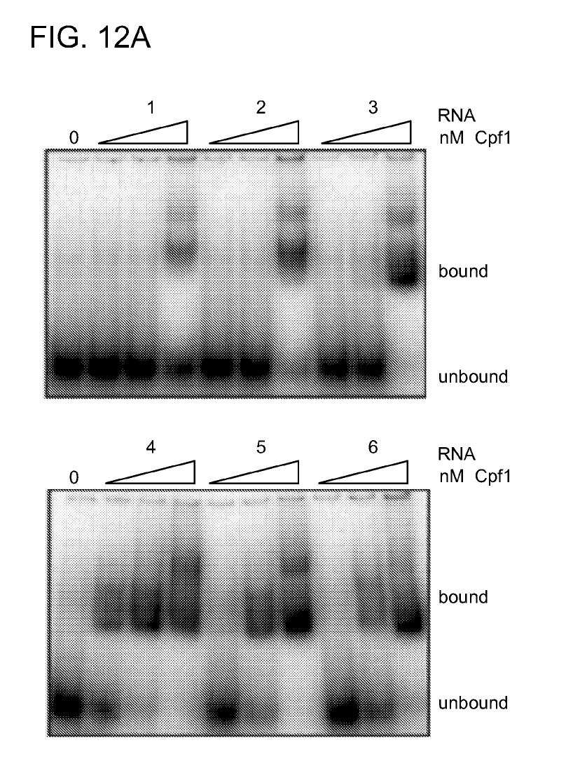

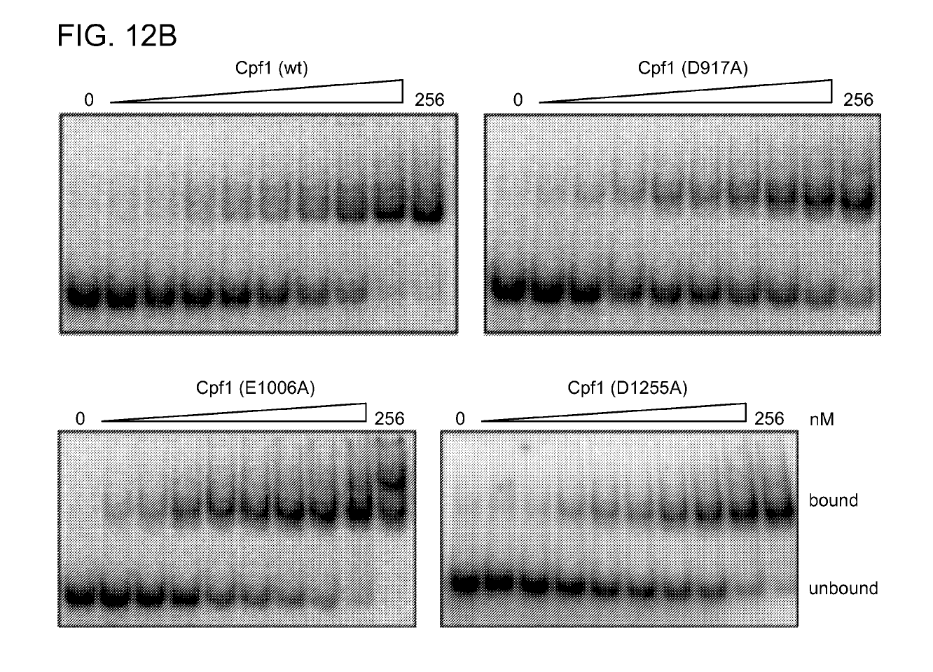

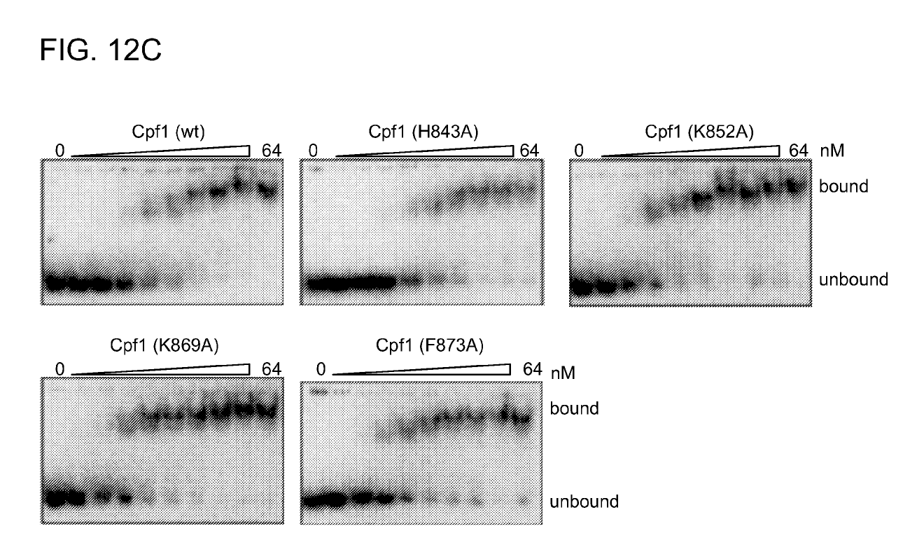

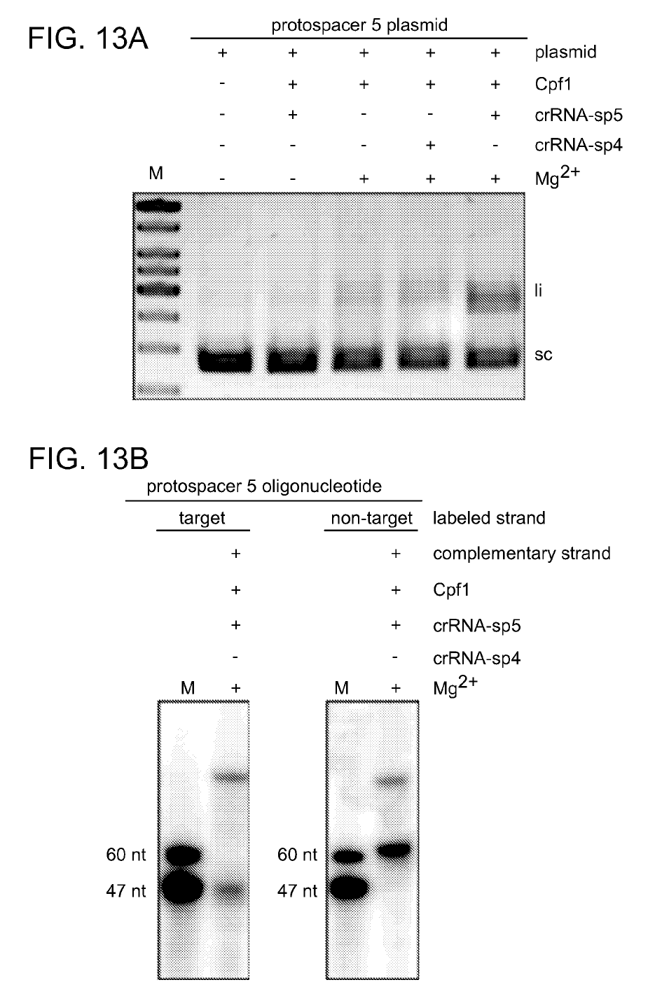

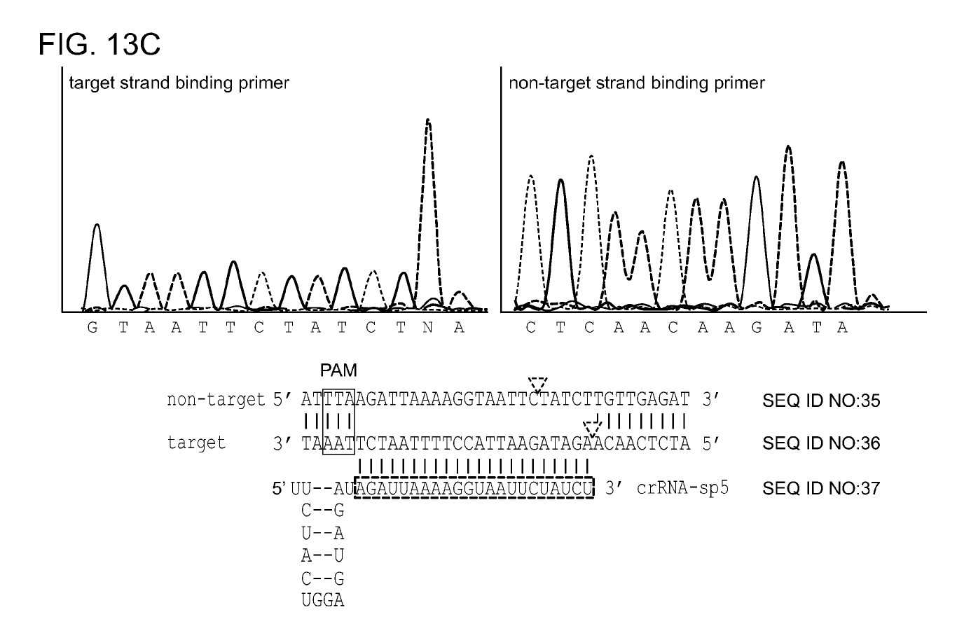

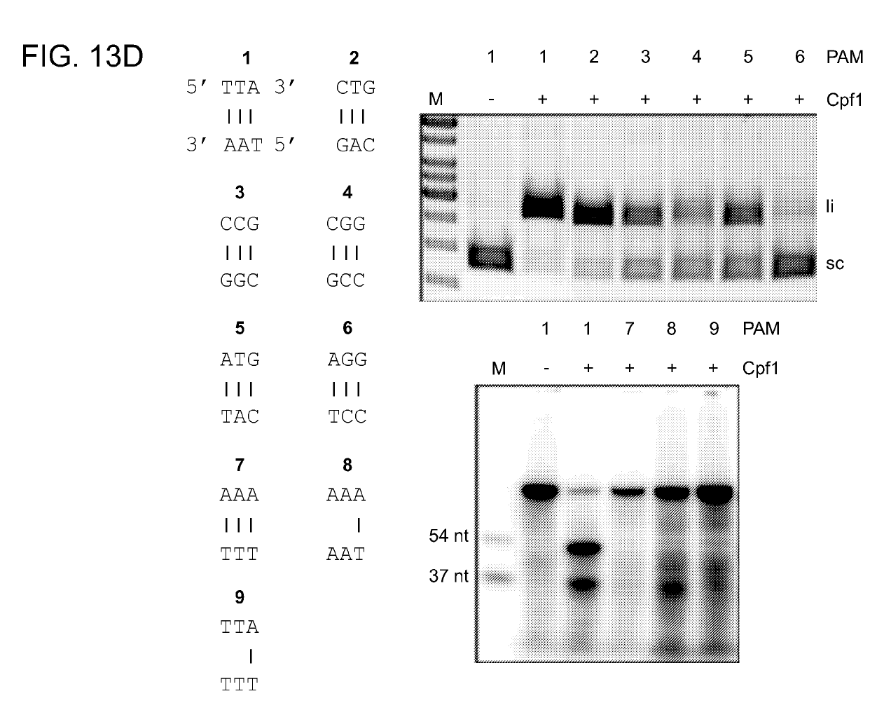

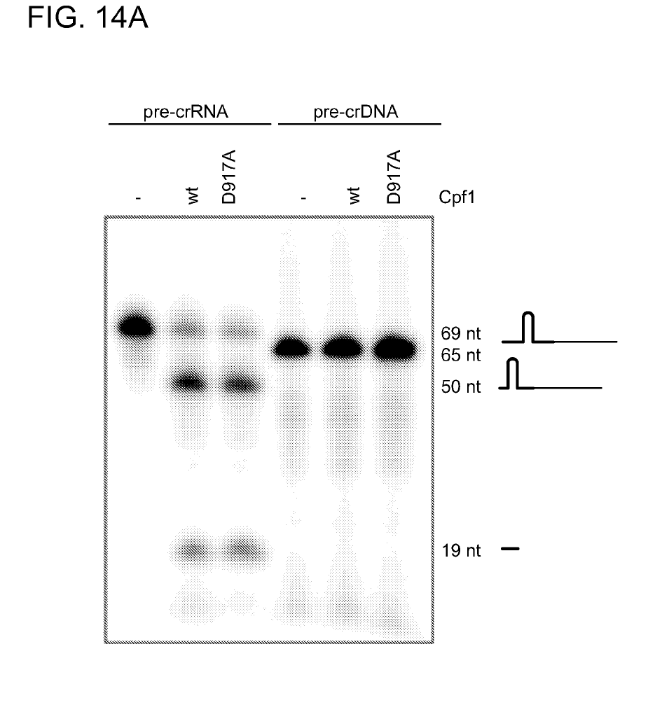

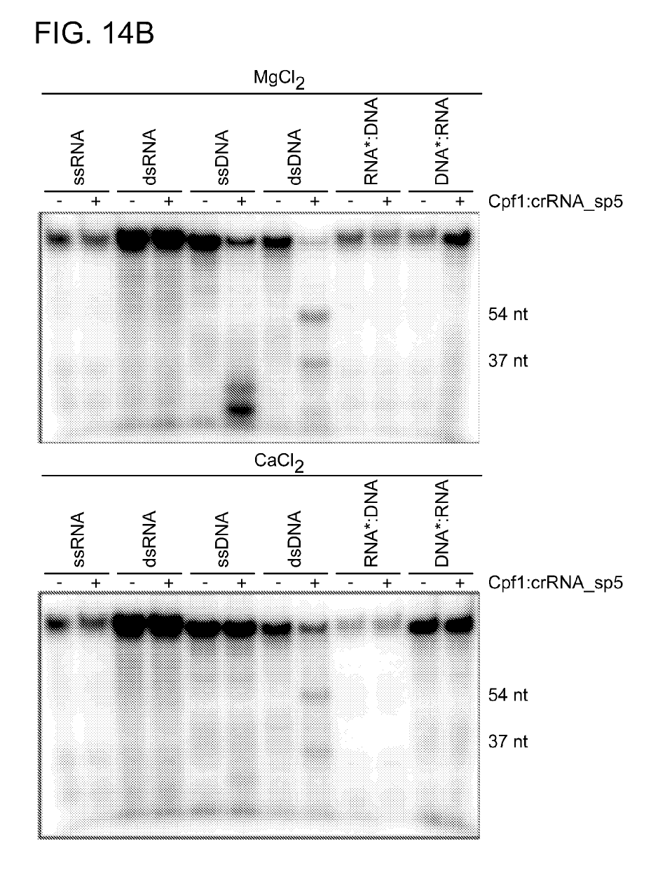

SEQ ID NOs:12, 13, and 14 are exemplary non-target, target DNA and mature crRNA shown in FIG. 3C.

SEQ ID NOs:15 provides an exemplary CRISPR array shown in FIG. 5B.

SEQ ID NOs:16, 17, 18, 19 provide structures various Cpf1 cleavage products which are represented schematically in FIG. 7.

SEQ ID NOs:20-26 represent various repeat variants of pre-crRNA-sp5 (pre-crRNA with spacer 5) with an altered repeat sequence, a destroyed repeat structure, single nucleotide exchanges (1-4) in the RRS and changed loop and stem sizes, as illustrated in FIGS. 9A-9C.

SEQ ID NOs:27, 28, 29, 30, 31, 32, 33, 34 provides RNA constructs shown in FIGS. 11C-11D.

SEQ ID NOs:35, 36, and 37 are sequences from the sequencing analysis illustrated in FIG. 13C.

SEQ ID NO:38 provides the amino acid sequence of Cpf1 encoded by SEQ ID NO:1.

SEQ ID NOs:39-49 are exemplary Protein Transduction Domains that could be used in conjugates.

SEQ ID NO:50 is an exemplary permeant peptide.

SEQ ID NOs:51-171 represent various oligonucleotides used in this study. The invention includes any of the sequences shown in the Sequence Listing and variants thereof as described in further detail in the Detailed Description.

### DETAILED DESCRIPTION

### Terminology

All technical and scientific terms used herein have the same meaning as commonly understood by one of ordinary skill in the art to which this invention belongs, unless the technical or scientific term is defined differently herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Genomic DNA” refers to the DNA of a genome of an organism including, but not limited to, the DNA of the genome of a bacterium, fungus, archea, plant or animal.

“Manipulating” DNA encompasses binding, nicking one strand, or cleaving (i.e., cutting) both strands of the DNA, or encompasses modifying the DNA or a polypeptide associated with the DNA. Manipulating DNA can silence, activate, or modulate (either increase or decrease) the expression of an RNA or polypeptide encoded by the DNA.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e., not include any mismatches.

By “hybridizable” or “complementary” or “substantially complementary” it is meant that a nucleic acid (e.g., RNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³M, less than 10⁻¹⁴ M, or less than 10⁻¹⁵ M. “Affinity” refers to the strength of binding, increased binding affinity being correlated with a lower K_{d}. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee, ebi.Ac.Uk/Tools/msa/muscle, mafft.cbrc/alignment/software [KL: check the website addresses]. See, e.g., Altschul et al. (1990), J. Mol. Biol. 215:403-10. Sequence alignments standard in the art are used according to the invention to determine amino acid residues in a Cpf1 ortholog that “correspond to” amino acid residues in another Cpf1 ortholog. The amino acid residues of Cpf1 orthologs that correspond to amino acid residues of other Cpf1 orthologs appear at the same position in alignments of the sequences.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a site-directed modifying polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca²⁺-calmodulin-dependent protein kinase II-alpha (CamKIM) promoter (see, e.g., Mayford et al. (1996) Proc. Natl. Acad. Sci. USA 93:13250; and Casanova et al. (2001) Genesis 31:37); a CMV enhancer/platelet-derived growth factor-p promoter (see, e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyiirek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., site-directed modifying polypeptide, or Cpf1 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to two components that are defined by structures derived from different sources. For example, where “chimeric” is used in the context of a chimeric polypeptide (e.g., a chimeric Cpf1 protein), the chimeric polypeptide includes amino acid sequences that are derived from different polypeptides. A chimeric polypeptide may comprise either modified or naturally-occurring polypeptide sequences (e.g., a first amino acid sequence from a modified or unmodified Cpf1 protein; and a second amino acid sequence other than the Cpf1 protein). Similarly, “chimeric” in the context of a polynucleotide encoding a chimeric polypeptide includes nucleotide sequences derived from different coding regions (e.g., a first nucleotide sequence encoding a modified or unmodified Cpf1 protein; and a second nucleotide sequence encoding a polypeptide other than a Cpf1 protein).

The term “chimeric polypeptide” refers to a polypeptide which is not naturally occurring, e.g., is made by the artificial combination (i.e., “fusion”) of two otherwise separated segments of amino sequence through human intervention. A polypeptide that comprises a chimeric amino acid sequence is a chimeric polypeptide. Some chimeric polypeptides can be referred to as “fusion variants.”

“Heterologous,” as used herein, means a nucleotide or peptide that is not found in the native nucleic acid or protein, respectively. For example, in a chimeric Cpf1 protein, the RNA-binding domain of a naturally-occurring bacterial Cpf1 polypeptide (or a variant thereof) may be fused to a heterologous polypeptide sequence (i.e., a polypeptide sequence from a protein other than Cpf1 or a polypeptide sequence from another organism). The heterologous polypeptide may exhibit an activity (e.g., enzymatic activity) that will also be exhibited by the chimeric Cpf1 protein (e.g., methyltransferase activity, acetyltransferase activity, kinase activity, ubiquitinating activity, etc.). A heterologous nucleic acid may be linked to a naturally-occurring nucleic acid (or a variant thereof) (e.g., by genetic engineering) to generate a chimeric polynucleotide encoding a chimeric polypeptide. As another example, in a fusion variant Cpf1 site-directed polypeptide, a variant Cpf1 site-directed polypeptide may be fused to a heterologous polypeptide (i.e., a polypeptide other than Cpf1), which exhibits an activity that will also be exhibited by the fusion variant Cpf1 site-directed polypeptide. A heterologous nucleic acid may be linked to a variant Cpf1 site-directed polypeptide (e.g., by genetic engineering) to generate a polynucleotide encoding a fusion variant Cpf1 site-directed polypeptide. “Heterologous,” as used herein, additionally means a nucleotide or polypeptide in a cell that is not its native cell.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence. The term “non-naturally occurring” includes molecules that are markedly different from their naturally occurring counterparts, including chemically modified or mutated molecules.

A “vector” or “expression vector” is a replicon, such as plasmid, phage, virus, or cosmid, to which another DNA segment, i.e., an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

A cell has been “genetically modified” or “transformed” or“transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell.

In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: S0169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian germ cell), by virtue of introduction into a suitable eukaryotic host cell of an exogenous nucleic acid.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-directed modifying polypeptide is used for targeted double-stranded DNA cleavage.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

By “site-directed polypeptide” or “RNA-binding site-directed polypeptide” or “RNA-binding site-directed polypeptide” it is meant a polypeptide that binds RNA and is targeted to a specific DNA sequence. A site-directed polypeptide as described herein is targeted to a specific DNA sequence by the RNA molecule to which it is bound. The RNA molecule comprises a sequence that is complementary to a target sequence within the target DNA, thus targeting the bound polypeptide to a specific location within the target DNA (the target sequence).

The RNA molecule that binds to the site-directed modifying polypeptide and targets the polypeptide to a specific location within the target DNA is referred to herein as the “guide RNA” or “guide RNA polynucleotide” (also referred to herein as a “guide RNA” or “gRNA”). A guide RNA comprises two segments, a “DNA-targeting segment” and a “protein-binding segment.” By “segment” it is meant a segment/section/region of a molecule, e.g., a contiguous stretch of nucleotides in an RNA. As an illustrative, non-limiting example, a protein-binding segment of a guide RNA can comprise base pairs 5-20 of the RNA molecule that is 40 base pairs in length; and the DNA-targeting segment can comprise base pairs 21-40 of the RNA molecule that is 40 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA (the complementary strand of the target DNA) designated the “protospacer-like” sequence herein. The protein-binding segment (or “protein-binding sequence”) interacts with a site-directed modifying polypeptide. When the site-directed modifying polypeptide is a Cpf1 or Cpf1 related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA.

The protein-binding segment of a guide RNA comprises, in part, two complementary stretches of nucleotides that hybridize to one another to form a double stranded RNA duplex (dsRNA duplex).

In some embodiments, a nucleic acid (e.g., a guide RNA, a nucleic acid comprising a nucleotide sequence encoding a guide RNA; a nucleic acid encoding a site-directed polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator-RNA with an aptamer may not be able to bind to the cognate targeter-RNA unless the aptamer is bound by the appropriate drug; (ii) a targeter-RNA with an aptamer may not be able to bind to the cognate activator-RNA unless the aptamer is bound by the appropriate drug; and (iii) a targeter-RNA and an activator-RNA, each comprising a different aptamer that binds a different drug, may not be able to bind to each other unless both drugs are present. As illustrated by these examples, a two-molecule guide RNA can be designed to be inducible.

Examples of aptamers and riboswitches can be found, for example, in: Nakamura et al., Genes Cells. 2012 May; 17(5):344-64; Vavalle et al., Future Cardiol. 2012 May; 8(3):371-82; Citartan et al., Biosens Bioelectron. 2012 Apr. 15; 34(1):1-11; and Liberman et al., Wiley Interdiscip Rev RNA. 2012 May-June; 3(3):369-84; all of which are herein incorporated by reference in their entirety.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “embryonic stem cell” (ESC) is meant a PSC that was isolated from an embryo, typically from the inner cell mass of the blastocyst. ESC lines are listed in the NIH Human Embryonic Stem Cell Registry, e.g., hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g., human, equine, bovine, porcine, canine, feline, rodent, e.g., mice, rats, hamster, primate, etc. (Thomson et al. (1998) Science 282:1145; Thomson et al. (1995) Proc. Natl. Acad. Sci. USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. No. 7,029,913, U.S. Pat. No. 5,843,780, and U.S. Pat. No. 6,200,806, the disclosures of which are incorporated herein by reference. Methods for proliferating hESCs in the undifferentiated form are described in WO 99/20741, WO 01/51616, and WO 03/020920. By “embryonic germ stem cell” (EGSC) or “embryonic germ cell” or “EG cell” is meant a PSC that is derived from germ cells and/or germ cell progenitors, e.g., primordial germ cells, i.e., those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, US Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e., it is no longer undergoing divisions. This quiescent state may be temporary, i.e., reversible, or it may be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA.

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., Harboor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The phrase “consisting essentially of” is meant herein to exclude anything that is not the specified active component or components of a system, or that is not the specified active portion or portions of a molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Genome Editing

Genome editing generally refers to the process of modifying the nucleotide sequence of a genome, preferably in a precise or predetermined manner. Examples of methods of genome editing described herein include methods of using site-directed nucleases to cut DNA at precise target locations in the genome, thereby creating double-strand or single-strand DNA breaks at particular locations within the genome. Such breaks can be and regularly are repaired by natural, endogenous cellular processes such as homology-directed repair (HDR) and non-homologous end-joining (NHEJ), as recently reviewed in Cox et al., Nature Medicine 21(2), 121-31 (2015). NHEJ directly joins the DNA ends resulting from a double-strand break sometimes with the loss or addition of nucleotide sequence which may disrupt or enhance gene expression. HDR utilizes a homologous sequence, or donor sequence, as a template for inserting a defined DNA sequence at the break point. The homologous sequence may be in the endogenous genome, such as a sister chromatid. Alternatively, the donor may be an exogenous nucleic acid such as a plasmid, a single-strand oligonucleotide, a duplex oligonucleotide or a virus, that has regions of high homology with the nuclease-cleaved locus, but which may also contain additional sequence or sequence changes including deletions that can be incorporated into the cleaved target locus. A third repair mechanism is microhomology-mediated end joining (MMEJ), also referred to as “Alternative NHEJ, in which the genetic outcome is similar to NHEJ in that small deletions and insertions can occur at the cleavage site. MMEJ makes use of homologous sequences of a few basepairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961(2015); Mateos-Gomez et al., Nature 518, 254-57 (2015); Ceccaldi et al., Nature 528, 258-62 (2015). In some instances it may be possible to predict likely repair outcomes based on analysis of potential microhomologies at the site of the DNA break.

Each of these genome editing mechanisms can be used to create desired genomic alterations. The first step in the genome editing process is to create typically one or two DNA breaks in the target locus as close as possible to the site of intended mutation. This can achieved via the use of site-directed polypeptides, as described and illustrated herein.

Site-directed polypeptides can introduce double-strand breaks or single-strand breaks in nucleic acid, (e.g., genomic DNA). The double-strand break can stimulate a cell's endogenous DNA-repair pathways (e.g., homology-dependent repair (HDR) and non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage and can lead to disruption or alteration of gene expression. HDR can occur when a homologous repair template, or donor, is available. The homologous donor template comprises sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide or viral nucleic acid. With exogenous donor templates it is common to introduce additional nucleic acid sequence (such as a transgene) or modification (such as a single base change or a deletion) between the flanking regions of homology so additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few basepairs flanking the cleavage site to drive a favored end-joining DNA repair outcome. In some instances it may be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

A. Guide RNA

For further detailed description of Cpf1, see section B below and elsewhere in this specification.

The present disclosure provides a guide RNA that directs the activities of an associated polypeptide (e.g., a site-directed modifying polypeptide) to a specific target sequence within a target DNA. A guide RNA comprises: a first segment (also referred to herein as a “DNA-targeting segment” or a “DNA-targeting sequence”) and a second segment (also referred to herein as a “protein-binding segment” or a “protein-binding sequence”). Both segments described generally below. The guide RNA is also known as a crRNA, and is derived from a pre-crRNA. The pre-crRNA may, but is not required to be, longer than the crRNA.

The DNA-targeting segment of a guide RNA comprises a nucleotide sequence that is complementary to a sequence in a target DNA. In other words, the DNA-targeting segment of a guide RNA interacts with a target DNA in a sequence-specific manner via hybridization (i.e., base pairing). As such, the nucleotide sequence of the DNA-targeting segment may vary and determines the location within the target DNA that the guide RNA and the target DNA will interact. The DNA-targeting segment of a guide RNA can be modified (e.g., by genetic engineering) to hybridize to any desired sequence within a target DNA.

The DNA-targeting segment can have a length of from about 20 nucleotides to about 22 nucleotides. In some cases, the DNA-targeting sequence of the DNA-targeting segment that is complementary to a target sequence of the target DNA is 20 nucleotides, 21 nucleotides, or 22 nucleotides in length

The percent complementarity between the DNA-targeting sequence of the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%) over the 20-22 nucleotides.

The protein-binding segment of a guide RNA interacts with a site-directed modifying polypeptide. The guide RNA guides the bound polypeptide to a specific nucleotide sequence within target DNA via the above mentioned DNA-targeting segment. The protein-binding segment of a guide RNA comprises two stretches of nucleotides that are complementary to one another. The complementary nucleotides of the protein-binding segment hybridize to form a double stranded RNA duplex (dsRNA), i.e., a stem-loop structure. The protein-binding segment of a guide RNA is about 20 (e.g., 19) nucleotides in length, which is comprised of a short sequence of about 4 nucleotides, and a repeat stem loop of about 12 nucleotides.

In vitro cleavage assays show that Cpf1 processes a pre-crRNA consisting of a full-length repeat-spacer, yielding a 19-nt repeat fragment, and a 50-nt repeat-spacer crRNA intermediate (FIG. 2). Only RNAs with full-length repeat sequences were processed, indicating that the RNA cleavage activity is repeat-dependent (FIG. 7). The observed cleavage site is in good agreement with the data obtained by RNA-seq (FIG. 5). The crRNAs produced in vitro represent intermediate forms that undergo further processing at the 5′ and 3′ ends by a nonspecific mechanism in vivo. Cpf1 cleaves pre-crRNA four nucleotides upstream of the stem-loop (FIG. 1). The cleavage site is reminiscent of many Cas6 enzymes and Cas5d, which recognize the hairpin of their respective repeats. Cpf1, however, does not cleave directly at the base of the stem-loop, suggesting that the structure is not the only requirement for processing of pre-crRNA. Northern blot analysis using an inducible Escherichia coli heterologous system also demonstrates processing of pre-crRNA upon Cpf1 expression (FIG. 8), resulting in the expected RNA fragments. To investigate the importance of the repeat and its hairpin structure in successful Cpf1 processing, we designed RNAs with mutations that yield either an altered repeat sequence keeping the stem-loop structure or an unstructured repeat. In contrast to the wild-type RNA substrate containing an intact repeat, none of the mutated RNAs was cleaved by Cpf1 (FIG. 9). We further designed repeat variants with either single nucleotide mutations between the cleavage site and the stem-loop (a region referred to as repeat recognition sequence (RRS)) or different sizes of the loop and stem regions (FIG. 9). Single nucleotide mutations in the RRS yielded repeat variants that were not, or only poorly, cleaved by Cpf1 (FIG. 9), indicating that these residues between the stem and the cleavage site have a role in processing of the substrate. This can be explained by the distinct secondary structure of crRNA in complex with Cpf1, where the RRS folds back to make contacts with the stem-loop. Changes in the loop region of the repeat structure resulted in reduced cleavage activity for a shorter loop, whereas an increased loop length did not influence cleavage (FIG. 9). Extensive contacts of Cpf1 to the stem-loop of the crRNA may explain why alterations of the stem structure yielded non-cleavable substrates. These results highlight the requirement of a stem-loop structure specific in length and sequence for recognition by Cpf1. Thus, the repeat cleavage reaction is highly sequence- and structure-dependent.

Accordingly, in some embodiments, a non-naturally occurring guide RNA is configured to target Cpf1 to a target site on double stranded DNA, wherein the gRNA is at least 69-nt long but no longer than 100 nt. A guide RNA can be configured to target Cpf1 to a target site on double stranded DNA, wherein the gRNA is capable of being cleaved by Cpf1 at 4 nt of upstream of stem-loop of a repeat and/or generating a repeat fragment (e.g., about 19-nt) and a mature form of crRNA which is 42-44 nt long. In some embodiments, gRNA is 42-44 nt long. In some embodiments, gRNA is configured to target Cpf1 to a target site on double stranded DNA and consists essentially of repeat-spacer-repeat.

Nucleic acids encoding gRNAs of the invention, and vectors comprising such nucleic acids are also provided herein.

B. Cpf1

Detection of small RNAs (sRNAs) expressed from a new CRISPR-Cas array led to the discovery of a new system associated with a cas gene called Cpf1 (previous nomenclature Fno) that is distinct from all cas genes identified so far. See FIG. 5A. The Type V-A CRISPR array contains a series of 9 spacer sequences separated by 36-nt repeat sequences. The mature RNAs are composed of repeat sequence in 5′ and spacer sequence in 3′, similar to the repeat-spacer composition of Type I and III systems, but distinct from the spacer-repeat composition of Types II systems. Similar to Type I systems, the repeat forms a hairpin structure located at the 3′ end of the repeat. Neither the presence of an anti-CRISPR repeat nor the expression of a tracrRNA homolog could be detected in the vicinity of the F. novicida Type V-A locus, indicating that Cpf1 uses a distinct mode of crRNA biogenesis compared to the already described mechanisms.

It was investigated whether Cpf1 acts as the single effector enzyme in pre-crRNA processing in type V-A systems. Recombinant F. novicida Cpf1 protein was overexpressed, purified and biochemically characterized. Naturally occurring site-directed modifying polypeptides binding a guide RNA, are thereby directed to a specific sequence within a target DNA, and cleave the target DNA to generate a double strand break. The nucleic acid sequence of the Francisella Cpf1 endonuclease is set out in SEQ ID NO:1. The corresponding amino acid sequence encoded by this nucleotide sequence is provided as SEQ ID NO:38. A site-directed modifying polypeptide comprises three portions, an RNA-binding portion, an RNase activity portion, and a DNase activity portion. In some embodiments, a site-directed modifying polypeptide comprises: (i) an RNA-binding portion that interacts with a guide RNA, wherein the guide RNA comprises a nucleotide sequence that is complementary to a sequence in a target DNA; (ii) an activity portion that exhibits site-directed enzymatic activity (e.g., activity for RNA cleavage), wherein the site of enzymatic activity is determined by the palindromic hairpin structures formed by the repeats of pre-crRNA and cleaves the pre-crRNA 4 nt upstream, the base of the hairpins generating intermediate forms of crRNAs (e.g., composed of repeat-spacer (5′-3′)); and (iii) an activity portion that exhibits site-directed enzymatic activity (e.g., activity for DNA cleavage), wherein the site of enzymatic activity is determined by the guide RNA.

Cpf1 is a monomer with a theoretical molecular weight of 153 kDa. Recombinant F. novicida Cpf1 protein was overexpressed and purified. Size-exclusion chromatography was performed to determine the oligomeric state of the protein. Analysis of the data revealed an apparent molecular weight of 187 kDa, indicating that Cpf1 is a monomer. The monomeric nature is consistent with Cpf1 forming a complex with the guide crRNA to bind and cleave target DNA because if the active protein were a dimer as reported by others, it would probably require a tandem DNA target site, or alternatively, two different crRNAs targeting the top and bottom strand of the DNA.

Cpf1 cleaves pre-crRNA at the level of the repeats. As with all CRISPR-Cas systems, the maturation of crRNAs occurs by a first cleavage taking place at the level of the repeats leading to the formation of intermediate forms of crRNAs that in some systems undergo additional processing/trimming events. Cpf1 differs fundamentally from type II systems in that a complex of Cpf1 and a single RNA, the crRNA, can cleave DNA without the presence of a second RNA (such as the tracrRNA required in type II Cas9 systems). Cpf1 was overexpressed and purified and used in an in vitro cleavage assay with various precursor forms of crRNAs. Only RNAs with full-length repeat sequences were processed, indicating that the RNA cleavage activity of Cpf1 is repeat-dependent. Northern Blot analysis using an inducible E. coli heterologous system also demonstrated processing of a pre-crRNA upon Cpf1 expression.

Cpf1 cleaves pre-crRNA 4 nucleotides upstream of the stem-loop. This is reminiscent to many Cas6 enzymes and Cas5d, which recognize the hairpin of their respective repeats. Cpf1, however, does not cleave directly at the base of the stem-loop, suggesting that the structure is not the only requirement for processing of pre-crRNA. RNAs with mutations that yield either an altered repeat sequence keeping the stem-loop structure or an unstructured repeat were designed. In contrast to wild type RNA substrate containing an intact repeat, none of the mutated RNAs were cleaved by Cpf1, indicating that the repeat cleavage reaction is sequence and structure dependent.

Cpf1 is a metal ion-dependent endoribonuclease. A variety of divalent metal ions were tested in RNA cleavage assays. The activity of Cpf1 in pre-crRNA processing was best when Mg²⁺ was added to the reaction. Supplementation with Ca²⁺, Mn²⁺ and Co²⁺ also mediated cleavage, however not to the level of specificity observed with Mg². This is in contrast to the ion-independent reaction of Cas6 enzymes (Types I and III) or Cas5d (Type I-C). This highlights a novel crRNA biogenesis mechanism in which Cpf1 is a metal-dependent endoribonuclease cleaving pre-crRNA in a sequence and structure specific manner. Thus, Cpf1 can therefore be “ionically modulated” by altering the relative levels of calcium and/or magnesium to which the protein is exposed.

Cpf1 also acts as a DNA endonuclease guided by crRNA to cleave dsDNA site-specifically. Only crRNA complementary to the target mediated Cpf1 DNA cleavage. To further analyze the RNA requirements for this activity, several RNAs containing various structures were constructed. Only RNAs with an intact stem-loop were able to mediate Cpf1 DNA cleavage activity.

DNA cleavage is also metal ion dependent. Remarkably, the studies herein show that in addition to Mg²⁺ and Mn²⁺, which were shown to mediate activity in Cas9, Cpf1 can cleave DNA also in presence of Ca²⁺. To investigate potential differences in cleavage with Mg²⁺ or Ca²⁺, DNA cleavage reactions were performed in the presence of either of these ions. In contrast to a recent publication showing that the HNH motif of Cas9 from Neisseria meningitidis is Ca²⁺ dependent, significant differences in target or non-target strand cleavage efficiency of Cpf1 in the presence of Ca²⁺ or Mg²⁺ were not observed. This indicates the presence of only one catalytic motif in Cpf1 that is responsible for cleaving both DNA strands and can coordinate Mg²⁺ as well as Ca²⁺ ions.

Cpf1 cleaves DNA via a staggered cut that produces a 5 nt 5′ overhang. Cleavage reactions using oligonucleotide duplexes with either radiolabeled target or non-target strand generated products of different sizes, which was confirmed by sequencing of plasmid cleavage products, that demonstrated a staggered cut by Cpf1 producing a 5 nt 5′ overhang.

C. Protospacer-Adjacent Motif (PAM)

Aligning the two predicted protospacer sequences of the F. novicida U112 type V-A CRISPR-Cas revealed a conserved 5′-TTA-3′ sequence located on the non-target strand upstream of the protospacer. To verify the potential PAM, protospacer 5 was cloned without its flanking region yielding a 5′-CTG-3′ sequence. Both plasmids were cleaved equally well by Cpf1, indicating that the second position in this sequence is critical (FIG. 3d, FIG. 14d). Mutagenesis of all three nucleotides followed by DNA cleavage analysis shows that Cpf1 recognizes a PAM, defined as 5′-YTN-3′, upstream of the crRNA-complementary DNA sequence on the non-target strand. This result expands on the already reported 5′-TTN-3′ PAM reported by Zetsche et al. (Cell, 2015, 163:759-771). To analyze strand specificity of PAM recognition, oligonucleotide substrates with either AAN or TTN on both strands were designed. These substrates were not cleaved by Cpf1, indicating that the PAM needs to be double-stranded and is probably recognized on both strands (FIG. 3D, lower panel). Accordingly, in some embodiments, the invention provides a non-naturally occurring guide RNA against a target DNA, said gRNA comprising a repeat (comprising a stem-loop structure) and a spacer, wherein the spacer comprises a sequence complementary to the sequence immediately adjacent upstream to complement of 5′-YTN-3′ on the non-target strand of the target DNA (or identical to the sequence immediately downstream of 5′-YTN-3′ on the non-target strand).

Cpf1 has a seed sequence of eight nucleotides proximal to the PAM. During interference of Type I and II systems the first 8-10 nt of the protospacer are crucial to enable the formation of a stable R-loop. This sequence is called seed sequence. Type II cleavage occurs 3 bp upstream of the PAM within the protospacer. In contrast, the PAM and cleavage site of Cpf1 lie on opposite sides of the protospacer. To analyze the length of the seed sequence, plasmids having single mismatches between spacer and protospacer along the target sequence were constructed. Cpf1 is sensitive to mismatches within the first 8 nucleotides on the PAM proximal side, while four consecutive mismatches are not tolerated. Furthermore, Cpf1 shows sensitivity to mismatches around the cleavage site (position 1-4 on the PAM distal site), however to a lesser extent. These results are in discrepancy to already published data showing a seed sequence of only 3-5 nucleotides PAM proximal, indicating that there might be other factors influencing the specificity, like the base content of the target sequence. These results indicate that Cpf1, similar to Cas9, first recognizes the PAM and then tests crRNA complementarity to the DNA target. Mismatches around the target site might disturb correct positioning of the catalytic residues and therefore reduce cleavage activity. Accordingly, in some embodiments, the invention provides a non-naturally occurring guide RNA, said guide RNA having one or more mutations within 8 PAM-proximal nts in the spacer but no more than 3 consecutive mutations and/or in 1-4 nts of PAM-distal site.

Without wishing to be bound by theory, it is believed that the mechanism of action of DNA targeting can involve one or more of the following activities. crRNA-guided Cpf1 screens the target DNA to identify a PAM. Upon base-pairing between the spacer sequence of crRNA and the protospacer sequence on the target DNA, an R-loop may be formed in parallel crRNA strand pairing. Cpf1 introduces the 5′ overhang double-stranded (ds) breaks in the target DNA at a defined distance, 20-22 nucleotides, from the PAM on the target strand and 15-17 nt from the PAM on the non-target strand. Cpf1 is expected to be dynamic modifying its conformation upon binding to pre-crRNA, and associated to crRNA, upon binding of target DNA and during the cleavage reaction. The nucleolytic activities of Cpf1 require sequence-specific and structure-dependent binding of the nuclease to the hairpin structure formed by the crRNA repeats and to a protospacer-adjacent (PAM) motif on the target DNA.

Cpf1 comprises a dual activity of RNA and DNA cleavage, and uses distinct active domains for each nuclease reaction. To determine the active motifs, mutagenesis of conserved residues along the Cpf1 amino acid sequence was performed. Alanine substitution of residues H843, K852, K869 and F873 had no effect on DNA cleavage activity but showed decreased in vitro RNA cleavage activity. Mutagenesis of D917, E1006 and D1255 in the split RuvC motif resulted in loss of DNA cleavage activity, but did not influence the RNA processing activity of Cpf1, nor did it affect binding affinity to the DNA target. See FIGS. 4D and 13B. To determine the active motifs, mutagenesis of conserved residues along the Cpf1 amino acid sequence were performed. FIG. 4D summarizes mutated residues, which impact one of the two catalytic activities. Alanine substitution of residues H843, K852, K869 and F873 had no effect on DNA cleavage activity (FIG. 4A, upper panel), but showed decreased in vitro RNA cleavage activity (FIG. 4A, middle panel). To further confirm their involvement in RNA processing in vivo, a heterologous E. coli assay co-expressing pre-crRNA (repeat-spacer-repeat) and Cpf1 or a variant thereof was set up. Northern Blot analysis was done with total RNA extracted after induced expression (FIG. 4A, lower panel). It seems that in the presence of Cpf1, crRNA was protected from degradation and therefore more abundant. Expression of Cpf1_wt results in the production of a distinct band of around 65 nt, which corresponds to a mature crRNA formed by two cleavage events within the repeats. In presence of Cpf1_H843A, this band was not present; however, two additional longer transcripts appeared due to a changed processing by this mutant, already seen in vitro (FIG. 4A, middle panel). Mutants K852A and K869A also showed the production of the 65 nt fragment, although with less intensity compared to the wild type and in addition to the two products of longer sizes. In vitro, these mutants showed almost no RNA processing. RNA-binding experiments with Cpf1 (K852A) and Cpf1 (K869A) (FIG. 12C) indicated a slightly higher affinity for RNA than wild-type Cpf1, which may explain the cleavage products observed in vivo. The residual activity of these Cpf1 mutants produces processed RNA, which is likely to be bound tighter to the protein and therefore better protected from degradation. Cpf1 (F873A) had reduced RNA cleavage activity in vitro, which could not be detected in vivo. Mutation of the aforementioned residues did not negatively affect RNA binding (FIG. 12C), indicating that the identified residues of Cpf1 are potentially responsible for RNA cleavage. Analysis of the co-crystal structure of Lachnospiraceae bacterium Cpf1 revealed that the identified residues are located in close proximity to the 5′ of the processed crRNA (Dong et al. (2016) Nature, 532(7600):522-6). Mutagenesis of D917, E1 006 and D1255 in the split RuvC motif resulted in loss of DNA cleavage activity (FIG. 4D, upper panel) (see also Zetsche et al. (2015) Cell, 163:759-771), but did not influence the RNA processing activity of Cpf1 (FIG. 4B, lower panel), nor did it affect binding affinity to the DNA target (FIG. 12B).

Cpf1 mutants display metal ion dependent differences in DNA cleavage. While screening for active site residues, significant differences in DNA cleavage for some mutants was observed, dependent on the metal ion present in the reaction. Mutants E920A, Y1024A, and D1227A showed no DNA cleavage in the presence of Ca²⁺, but wild type activity when Mg²⁺ was present. Mutating residue E1028 also leads to loss of Ca²⁺ dependent cleavage and additionally decreases cleavage of the non-target strand in the presence of Mg²⁺, indicative of an involvement in non-target strand cleavage. In contrast, mutation of residues H922 and Y925 resulted in drastically decreased cleavage of the target strand in the presence of Ca²⁺. These mutants showed wild type levels of DNA cleavage activity in the presence of Mg²⁺. This suggests an involvement in Ca²⁺ coordination and target strand cleavage. Cpf1 can therefore be “ionically modulated” by altering the relative levels of calcium and/or magnesium to which the protein is exposed. Structural modifications can also be used to further modulate Cpf1. By inactivating the endonuclease activity of Cpf1 through mutations affecting the enzymatic activity, the protein can also be used to bind sequence-specifically without cleaving the DNA.

Two aspartates (D917, D1255) and one glutamate (E1006) form the catalytic site of Cpf1, which is in good agreement with other RuvC/RNaseH motifs. These kinds of catalytic motifs generally employ a two-metal-ion mechanism for DNA cleavage. Enzymes with a two-metal-ion mechanism are more stringent in the choice of the metal ion, with mostly a preference for Mg²⁺. In contrast, enzymes using a one-metal-ion mechanism for cleavage, like HNH nucleases, can be more flexible in their choice of metal ions. For example, Kpnl cleaves DNA with high fidelity in the presence of Ca²⁺, but more unspecifically in the presence of Mg²⁺. Cpf1 may also represent a new type of DNA-nuclease using two-metal-ion catalysis with the ability to utilize Mg²⁺ or Ca²⁺ ions.

Cpf1 is an enzyme with dual nucleolytic activity against RNA and DNA. Cpf1 is an enzyme that cleaves RNA in a highly sequence and structure dependent manner, and also performs specific DNA cleavage only in presence of the produced guide RNA. In context of CRISPR immunity, type V-A is the most efficient system described so far, utilizing only one enzyme, Cpf1, to process crRNA and to use this RNA to specifically target invading DNA. Cpf1 differs fundamentally from type II systems in that a complex of Cpf1 and a single RNA, the crRNA, can cleave DNA without the presence of a second RNA (such as the tracrRNA required in type II Cas9 systems).

Cpf1 can also be used to form a chimeric binding protein in which other domains and activities are introduced. By way of illustration, a Fokl domain can be fused to a Cpf1 protein, which can contain a catalytically active endonuclease domain, or a Fokl domain can be fused to a Cpf1 protein, which has been modified to render the Cpf1 endonuclease domain inactive. Other domains that can be fused to make chimeric proteins with Cpf1 including transcriptional modulators, epigenetic modifiers, tags and other labels or imaging agents, histones, and/or other modalities known in the art that modulate or modify the structure or activity of gene sequences.

Based on the sequence, and with reference to the structural specificity of binding of Cpf1 to the hairpin structures of crRNA forms, Cpf1 orthologues can be identified and characterized based on sequence similarities to the present system, as has been described with type II systems for example. For example, orthologs of Cpf1 include F. novicida U112, Prevotella albensis, Acidaminococcus sp. BV3L6, Eubacterium eligens CAG:72, Butyrivibrio fibrisolvens, Smithella sp. SCADC, Flavobacterium sp. 316, Porphyromonas crevioricanis, or Bacteroidetes oral taxon 274.

Exemplary Site-Directed Modifying Polypeptides

The invention provides an isolated, e.g., purified, non-naturally occurring Cpf1 polypeptide which comprises an amino acid sequence having at least about 75%, at least about 80%, at least about 85%, at least about 90%, at least about 95%, at least about 99%, or 100%, amino acid sequence identity to the sequence of SEQ ID NO:38 or any of amino acid sequences of SEQ ID NO:2-10. In some embodiments, the Cpf1 polypeptide is selected from the group selected from the following species: Fno, Fal, Asp, Eel, Bfi, SSp, Fsp, cPcr, and Bcr. In some embodiments, such a side-directed modifying polypeptide retains a) the capability of biding to a targeted site and, optionally, b) retains its activity. In some embodiments, the activity being retained is endoribonuclease and/or endonuclease activity. In certain embodiments, wherein the endonuclease activity does not require tracrRNA. In certain embodiments, the polypeptide is capable of processing pre-crRNA into mature forms of crRNA that direct target-specific binding of Cpf1 to target DNA.

In some embodiments, the RNase and/or DNase activity of the site-directed modifying polypeptide is altered relative to the wild type. The invention also provided a purified or isolated RNase domain of Cpf1, for example, comprising mutations in H843, K852, K869 or F873. The invention further provides purified or isolated DNase domain of Cpf1, for example, comprising mutations in D917, E1006 and/or D1255. The invention also provide a mutated domain or Cpf1 polypeptide, active in a monomeric form.

Additionally, the invention provides isolated DNA encoding the site-directed modifying of the invention, including the Cpf1 polypeptide, its mutated form or altered forms, or one of its nuclease active domains.

Nucleic Acid Modifications

In some embodiments, polynucleotides introduced into cells comprise one or more modifications which can be used, for example, to enhance activity, stability or specificity, alter delivery, reduce innate immune responses in host cells, or for other enhancements, as further described herein and known in the art.

In certain embodiments, modified polynucleotides are used in the CRISPR-Cas system, in which case the guide RNAs and/or a DNA or an RNA encoding a Cas endonuclease introduced into a cell can be modified, as described and illustrated below. Such modified polynucleotides can be used in the CRISPR-Cas system to edit any one or more genomic loci.

Using the CRISPR-Cas system for purposes of nonlimiting illustrations of such uses, modifications of guide RNAs can be used to enhance the formation or stability of the CRISPR-Cas genome editing complex comprising guide RNAs and a Cas endonuclease such as Cpf1. Modifications of guide RNAs can also or alternatively be used to enhance the initiation, stability or kinetics of interactions between the genome editing complex with the target sequence in the genome, which can be used for example to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half life in the cell to be increased. Modifications enhancing guide RNA half life can be particularly useful in embodiments in which a Cas endonuclease such as a Cpf1 is introduced into the cell to be edited via an RNA that needs to be translated in order to generate Cpf1 endonuclease, since increasing the half of guide RNAs introduced at the same time as the RNA encoding the endonuclease can be used to increase the time that the guide RNAs and the encoded Cas endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half life of the RNA and/or the elicitation of cytokines or other factors associated with immune responses.

One or more types of modifications can also be made to RNAs encoding an endonuclease such as Cpf1 that are introduced into a cell, including, without limitation, modifications that enhance the stability of the RNA (such as by decreasing its degradation by RNases present in the cell), modifications that enhance translation of the resulting product (i.e., the endonuclease), and/or modifications that decrease the likelihood or degree to which the RNAs introduced into cells elicit innate immune responses.

Combinations of modifications, such as the foregoing and others, can likewise be used. In the case of CRISPR-Cas, for example, one or more types of modifications can be made to guide RNAs (including those exemplified above), and/or one or more types of modifications can be made to RNAs encoding Cas endonuclease (including those exemplified above).

By way of illustration, guide RNAs used in the CRISPR-Cas system, or other smaller RNAs can be readily synthesized by chemical means, enabling a number of modifications to be readily incorporated, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating chemically-modified RNAs of greater length is to produce two or more molecules that are ligated together. Much longer RNAs, such as those encoding a Cpf1 endonuclease, are more readily generated enzymatically. While fewer types of modifications are generally available for use in enzymatically produced RNAs, there are still modifications that can be used to, e.g., enhance stability, reduced the likelihood or degree of innate immune response, and/or enhance other attributes, as described further below and in the art; and new types of modifications are regularly being developed.

By way of illustration of various types of modifications, especially those used frequently with smaller chemically synthesized RNAs, modifications can comprise one or more nucleotides modified at the 2′ position of the sugar, in some embodiments a 2′-O-alkyl, 2′-O-alkyl-O-alkyl or 2′-fluoro-modified nucleotide. In some embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, —N(CH₃)—O—CH₂ (known as a methylene(methylimino) or MMI backbone), CH₂—O—N(CH₃)—CH₂, CH₂—N(CH₃)—N(CH₃)—CH₂ and O—N(CH₃)— CH₂—CH₂ backbones; amide backbones [see De Mesmaeker et al, Ace. Chem. Res., 28:366-374 (1995)]; morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Braasch and David Corey, Biochemistry, 41(14): 4503-4510 (2002); Genesis, Volume 30, Issue 3, (2001); Heasman, Dev. Biol., 243: 209-214 (2002); Nasevicius et al., Nat. Genet., 26:216-220 (2000); Lacerra et al., Proc. Natl. Acad. Sci., 97: 9591-9596 (2000); and U.S. Pat. No. 5,034,506, issued Jul. 23, 1991.

Cyclohexenyl nucleic acid oligonucleotide mimetics are described in Wang et al., J. Am. Chem. Soc., 122: 8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃, OCH₃O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some embodiments, a modification includes 2′-methoxyethoxy (2′-O—CH₂CH₂OCH₃, also known as 2′-O-(2-methoxyethyl)) (Martin et al, Helv. Chim. Acta, 1995, 78, 486). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262. Further teaching of PNA compounds can be found in Nielsen et al, Science, 254: 1497-1500 (1991).

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, pp 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by 0.6-1.2 degrees C. (Sanghvi, Y. S., in Crooke, S. T. and Lebleu, B., eds., Antisense Research and Applications, CRC Press, Boca Raton, 1993, pp. 276-278) and are embodiments of base substitutions.

Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandte Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and —O-6 substituted purines, comprising 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, ‘Antisense Research and Applications’, CRC Press, Boca Raton, 1993, pp. 276-278) and are embodiments of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and US Patent Application Publication 20030158403.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, the guide RNAs and/or mRNA (or DNA) encoding an endonuclease such as Cpf1 are chemically linked to one or more moieties or conjugates that enhance the activity, cellular distribution, or cellular uptake of the oligonucleotide. Such moieties comprise but are not limited to, lipid moieties such as a cholesterol moiety [Letsinger et al., Proc. Natl. Acad. Sci. USA, 86: 6553-6556 (1989)]; cholic acid [Manoharan et al., Bioorg. Med. Chem. Let., 4: 1053-1060 (1994)]; a thioether, e.g., hexyl-S-tritylthiol [Manoharan et al, Ann. N. Y. Acad. Sci., 660: 306-309 (1992) and Manoharan et al., Bioorg. Med. Chem. Let., 3: 2765-2770 (1993)]; a thiocholesterol [Oberhauser et al., Nucl. Acids Res., 20: 533-538 (1992)]; an aliphatic chain, e.g., dodecandiol or undecyl residues [Kabanov et al., FEBS Lett., 259: 327-330 (1990) and Svinarchuk et al., Biochimie, 75: 49-54 (1993)]; a phospholipid, e.g., di-hexadecyl-rac-glycerol or triethylammonium 1,2-di-O-hexadecyl-rac-glycero-3-H-phosphonate [Manoharan et al., Tetrahedron Lett., 36: 3651-3654 (1995) and Shea et al., Nucl. Acids Res., 18: 3777-3783 (1990)]; a polyamine or a polyethylene glycol chain [Mancharan et al., Nucleosides & Nucleotides, 14: 969-973 (1995)]; adamantane acetic acid [Manoharan et al., Tetrahedron Lett., 36: 3651-3654 (1995)]; a palmityl moiety [(Mishra et al., Biochim. Biophys. Acta, 1264: 229-237 (1995)]; or an octadecylamine or hexylamino-carbonyl-t oxycholesterol moiety [Crooke et al., J. Pharmacol. Exp. Ther., 277: 923-937 (1996)]. See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., Protein Pept Lett. 21(10):1025-30 (2014). Other systems known in the art and regularly developed can be used to target biomolecules of use in the present case and/or complexes thereof to particular target cells of interest.

These targeting moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium l,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Longer polynucleotides that are less amenable to chemical synthesis and are typically produced by enzymatic synthesis can also be modified by various means. Such modifications can include, for example, the introduction of certain nucleotide analogs, the incorporation of particular sequences or other moieties at the 5′ or 3′ ends of molecules, and other modifications. By way of illustration, the mRNA encoding Cpf1 is approximately 4 kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that is often observed in cells following introduction of exogenous RNAs, particularly longer RNAs such as that encoding Cpf1.

Numerous such modifications have been described in the art, such as polyA tails, 5′ cap analogs (e.g., Anti Reverse Cap Analog (ARCA) or m7G(5′)ppp(5′)G (mCAP)), modified 5′ or 3′ untranslated regions (UTRs), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

There are numerous commercial suppliers of modified RNAs, including for example, TriLink Biotech, AxoLabs, Bio-Synthesis Inc., Dharmacon and many others. As described by TriLink, for example, 5-Methyl-CTP can be used to impart desirable characteristics such as increased nuclease stability, increased translation or reduced interaction of innate immune receptors with in vitro transcribed RNA. 5′-Methylcytidine-5′-Triphosphate (5-Methyl-CTP), N6-Methyl-ATP, as well as Pseudo-UTP and 2-Thio-UTP, have also been shown to reduce innate immune stimulation in culture and in vivo while enhancing translation as illustrated in publications by Kormann et al. and Warren et al. referred to below.

It has been shown that chemically modified mRNA delivered in vivo can be used to achieve improved therapeutic effects; see, e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-Methyl-A, 2-Thio-U and 5-Methyl-C, it was found substituting just one quarter of the uridine and cytidine residues with 2-Thio-U and 5-Methyl-C respectively, resulted in a significant decrease in toll-like receptor (TLR) mediated recognition of the mRNA in mice. By reducing the activation of the innate immune system, these modifications can therefore be used to effectively increase the stability and longevity of the mRNA in vivo; see, e.g., Kormann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7(5):618-30 (2010). Such modified mRNAs that act as primary reprogramming proteins can be an efficient means of reprogramming multiple human cell types. Such cells are referred to as induced pluripotency stem cells (iPSCs), and it was found that enzymatically synthesized RNA incorporating 5-Methyl-CTP, Pseudo-UTP and an Anti Reverse Cap Analog (ARCA) could be used to effectively evade the cell's antiviral response; see, e.g., Warren et al., supra.

Other modifications of polynucleotides described in the art include, for example, the use of polyA tails, the addition of 5′ cap analogs (such as m7G(5′)ppp(5′)G (mCAP)), modifications of 5′ or 3′ untranslated regions (UTRs), or treatment with phosphatase to remove 5′ terminal phosphates—and new approaches are regularly being developed.

A number of compositions and techniques applicable to the generation of modified RNAs for use herein have been developed in connection with the modification of RNA interference (RNAi), including small-interfering RNAs (siRNAs). siRNAs present particular challenges in vivo because their effects on gene silencing via mRNA interference are generally transient, which can require repeat administration. In addition, siRNAs are double-stranded RNAs (dsRNA) and mammalian cells have immune responses that have evolved to detect and neutralize dsRNA, which is often a by-product of viral infection. Thus, there are mammalian enzymes such as PKR (dsRNA-responsive kinase), and potentially retinoic acid-inducible gene I (RIG-I), that can mediate cellular responses to dsRNA, as well as Toll-like receptors (such as TLR3, TLR7 and TLR8) that can trigger the induction of cytokines in response to such molecules; see, e.g., the reviews by Angart et al., Pharmaceuticals (Basel) 6(4): 440-468 (2013); Kanasty et al., Molecular Therapy 20(3): 513-524 (2012); Burnett et al., Biotechnol J. 6(9):1130-46 (2011); Judge and MacLachlan, Hum Gene Ther 19(2):111-24 (2008); and references cited therein.

A large variety of modifications have been developed and applied to enhance RNA stability, reduce innate immune responses, and/or achieve other benefits that can be useful in connection with the introduction of polynucleotides into human cells as described herein; see, e.g., the reviews by Whitehead K A et al., Annual Review of Chemical and Biomolecular Engineering, 2: 77-96 (2011); Gaglione and Messere, Mini Rev Med Chem, 10(7):578-95 (2010); Chernolovskaya et al, Curr Opin Mol Ther., 12(2):158-67 (2010); Deleavey et al., Curr Protoc Nucleic Acid Chem Chapter 16:Unit 16.3 (2009); Behlke, Oligonucleotides 18(4):305-19 (2008); Fucini et al., Nucleic Acid Ther 22(3): 205-210 (2012); Bremsen et al., Front Genet 3:154 (2012).

As noted above, there are a number of commercial suppliers of modified RNAs, many of which have specialized in modifications designed to improve the effectiveness of siRNAs. A variety of approaches are offered based on various findings reported in the literature. For example, Dharmacon notes that replacement of a non-bridging oxygen with sulfur (phosphorothioate, PS) has been extensively used to improve nuclease resistance of siRNAs, as reported by Kole, Nature Reviews Drug Discovery 11:125-140 (2012). Modifications of the 2′-position of the ribose have been reported to improve nuclease resistance of the internucleotide phosphate bond while increasing duplex stability (Tm), which has also been shown to provide protection from immune activation. A combination of moderate PS backbone modifications with small, well-tolerated 2′-substitutions (2′-O-Methyl, 2′-Fluoro, 2′-Hydro) has been associated with highly stable siRNAs for applications in vivo, as reported by Soutschek et al. Nature 432:173-178 (2004); and 2′-O-Methyl modifications have been reported to be effective in improving stability as reported by Volkov, Oligonucleotides 19:191-202 (2009). With respect to decreasing the induction of innate immune responses, modifying specific sequences with 2′-O-Methyl, 2′-Fluoro, 2′-Hydro have been reported to reduce TLR7/TLR8 interaction while generally preserving silencing activity; see, e.g., Judge et al., Mol. Ther. 13:494-505 (2006); and Cekaite et al., J. Mol. Biol. 365:90-108 (2007). Additional modifications, such as 2-thiouracil, pseudouracil, 5-methylcytosine, 5-methyluracil, and N6-methyladenosine have also been shown to minimize the immune effects mediated by TLR3, TLR7, and TLR8; see, e.g., Kariko, K. et al., Immunity 23:165-175 (2005).

As is also known in the art, and commercially available, a number of conjugates can be applied to polynucleotides such as RNAs for use herein that can enhance their delivery and/or uptake by cells, including for example, cholesterol, tocopherol and folic acid, lipids, peptides, polymers, linkers and aptamers; see, e.g., the review by Winkler, Ther. Deliv. 4:791-809 (2013), and references cited therein.

Mimetics

A nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units, which gives PNA an amide containing backbone. The heterocyclic base moieties are bound directly or indirectly to aza nitrogen atoms of the amide portion of the backbone. Representative US patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are nonionic mimics of oligonucleotides, which are less likely to form undesired interactions with cellular proteins (Dwaine A. Braasch and David R. Corey, Biochemistry, 2002, 41(14), 45034510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 85958602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂-), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Modified Sugar Moieties

A nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)CH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy 2′-O—CH₂CH₂OCH₃, also known as -2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O—CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

Base Modifications and Substitutions

A nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

“Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

Conjugates

Another possible modification of a nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 36513654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of an exogenous polypeptide (e.g., a site-directed modifying polypeptide). In some embodiments, a PTD is covalently linked to the carboxyl terminus of an exogenous polypeptide (e.g., a site-directed modifying polypeptide). In some embodiments, a PTD is covalently linked to a nucleic acid (e.g., a guide RNA, a polynucleotide encoding a guide RNA, a polynucleotide encoding a site-directed modifying polypeptide, etc.). Exemplary PTDs include but are not limited to a minimal undecapeptide protein transduction domain (corresponding to residues 47-57 of HIV-1 TAT comprising YGRKKRRQRRR (SEQ ID NO:39); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); GVVTLNSAGYLLGKINLKALAALAKKIL (SEQ ID NO:40); KALAWEAKLAKALAKALAKHLAKALAKALKCEA (SEQ ID NO:41); and RQIKIWFQNRRMKWKK (SEQ ID NO:42). Exemplary PTDs include but are not limited to, YGRKKRRQRRR(SEQ ID NO:43); RKKRRQRRR (SEQ ID NO:44); an arginine homopolymer of from 3 arginine residues to 50 arginine residues; Exemplary PTD domain amino acid sequences include, but are not limited to, any of the following: YGRKKRRQRRR (SEQ ID NO:45); RKKRRQRR (SEQ ID NO:46); YARAAARQARA (SEQ ID NO:47); THRLPRRRRRR (SEQ ID NO:48); and GGRRARRRRRR (SEQ ID NO:49). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

Nucleic Acids Encoding a Guide RNA and/or a Site-Directed Modifying Polypeptide

The present disclosure provides a nucleic acid comprising a nucleotide sequence encoding a guide RNA and/or a site-directed modifying polypeptide. In some embodiments, a guide RNA-encoding nucleic acid is an expression vector, e.g., a recombinant expression vector.

In some embodiments, a method involves contacting a target DNA or introducing into a cell (or a population of cells) one or more nucleic acids comprising nucleotide sequences encoding a guide RNA and/or a site-directed modifying polypeptide. In some embodiments a cell comprising a target DNA is in vitro. In some embodiments a cell comprising a target DNA is in vivo. Suitable nucleic acids comprising nucleotide sequences encoding a guide RNA and/or a site-directed modifying polypeptide include expression vectors, where an expression vector comprising a nucleotide sequence encoding a guide RNA and/or a site-directed modifying polypeptide is a “recombinant expression vector.”

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell. Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding a guide RNA and/or a site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a guide RNA and/or a site-directed modifying polypeptide is operably linked to multiple control elements that allow expression of the nucleotide sequence encoding a guide RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, green fluorescent protein, etc.) that are fused to the site-directed modifying polypeptide, thus resulting in a chimeric polypeptide.

In some embodiments, a nucleotide sequence encoding a guide RNA and/or a site-directed modifying polypeptide is operably linked to an inducible promoter. In some embodiments, a nucleotide sequence encoding a guide RNA and/or a site-directed modifying polypeptide is operably linked to a constitutive promoter.

Methods of introducing a nucleic acid into a host cell are known in the art, and any known method can be used to introduce a nucleic acid (e.g., an expression construct) into a cell. Suitable methods include, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: S0169-409X(12)00283-9. doi: 10.1016/j.addr.2012.09.023), and the like.

Chimeric Polypeptides

The present disclosure provides a chimeric site-directed modifying polypeptide. A chimeric site-directed modifying polypeptide interacts with (e.g., binds to) a guide RNA (described above). The guide RNA guides the chimeric site-directed modifying polypeptide to a target sequence within target DNA (e.g., a chromosomal sequence or an extrachromosomal sequence, e.g., an episomal sequence, a minicircle sequence, a mitochondrial sequence, a chloroplast sequence, etc.). A chimeric site-directed modifying polypeptide modifies target DNA (e.g., cleavage or methylation of target DNA) and/or a polypeptide associated with target DNA (e.g., methylation or acetylation of a histone tail).

A chimeric site-directed modifying polypeptide modifies target DNA (e.g., cleavage or methylation of target DNA) and/or a polypeptide associated with target DNA (e.g., methylation or acetylation of a histone tail). A chimeric site-directed modifying polypeptide is also referred to herein as a “chimeric site-directed polypeptide” or a “chimeric RNA binding site-directed modifying polypeptide.”

A chimeric site-directed modifying polypeptide comprises two portions, an RNA-binding portion and an activity portion. A chimeric site-directed modifying polypeptide comprises amino acid sequences that are derived from at least two different polypeptides. A chimeric site-directed modifying polypeptide can comprise modified and/or naturally occurring polypeptide sequences (e.g., a first amino acid sequence from a modified or unmodified Cpf1 protein; and a second amino acid sequence other than the Cpf1 protein).

RNA-Binding Portion

In some cases, the RNA-binding portion of a chimeric site-directed modifying polypeptide is a naturally occurring polypeptide. In other cases, the RNA-binding portion of a chimeric site-directed modifying polypeptide is not a naturally occurring molecule (modified, e.g., mutation, deletion, insertion). Naturally occurring RNA-binding portions of interest are derived from site-directed modifying polypeptides known in the art. For example, FIG. 1 is a naturally occurring Cpf1 endonuclease that can be used as a site-directed modifying polypeptide. In some cases, the RNA-binding portion of a chimeric site-directed modifying polypeptide comprises an amino acid sequence having at least about 75%, at least about 80%, at least about 85%, at least about 90%, at least about 95%, at least about 98%, at least about 99%, or 100%, amino acid sequence identity to the RNA-binding portion of a polypeptide set forth in FIG. 1.

Activity Portion

In addition to the RNA-binding portion, the chimeric site-directed modifying polypeptide comprises an “activity portion.” In some embodiments, the activity portion of a chimeric site-directed modifying polypeptide comprises the naturally-occurring activity portion of a site-directed modifying polypeptide (e.g., Cpf1 endonuclease). In other embodiments, the activity portion of a subject chimeric site-directed modifying polypeptide comprises a modified amino acid sequence (e.g., substitution, deletion, insertion) of a naturally-occurring activity portion of a site-directed modifying polypeptide. Naturally-occurring activity portions of interest are derived from site-directed modifying polypeptides known in the art. For example, FIG. 1 is a naturally occurring Cpf1 endonucleases that can be used as a site-directed modifying polypeptide. The activity portion of a chimeric site-directed modifying polypeptide is variable and may comprise any heterologous polypeptide sequence that may be useful in the methods disclosed herein. In some embodiments, the activity portion of a site-directed modifying polypeptide comprises a portion of a Cpf1 ortholog that is at least 90% identical to activity portion amino acids of FIG. 1. In some embodiments, a chimeric site-directed modifying polypeptide comprises: (i) an RNA-binding portion that interacts with a guide RNA, wherein the guide RNA comprises a nucleotide sequence that is complementary to a sequence in a target DNA; (ii) an activity portion that exhibits site-directed enzymatic activity (e.g., activity for RNA cleavage), wherein the site of enzymatic activity is determined by the palindromic hairpin structures formed by the repeats of pre-crRNA and cleaves the pre-crRNA 4 nt upstream of the hairpins generating intermediate forms of crRNAs composed of repeat spacer (5′-3′); and (iii) an activity portion that exhibits site-directed enzymatic activity (e.g., activity for DNA cleavage), wherein the site of enzymatic activity is determined by the guide RNA.

Exemplary Chimeric Site-Directed Modifying Polypeptides

In some embodiments, the activity portion of the chimeric site-directed modifying polypeptide comprises a modified form of the Cpf1 protein, including modified forms of any of the Cpf1 orthologs. In some instances, the modified form of the Cpf1 protein comprises an amino acid change (e.g., deletion, insertion, or substitution) that reduces the naturally occurring nuclease activity of the Cpf1 protein. For example, in some instances, the modified form of the Cpf1 protein has less than 50%, less than 40%, less than 30%, less than 20%, less than 10%, less than 5%, or less than 1% of the nuclease activity of the corresponding wild-type Cpf1 polypeptide. In some cases, the modified form of the Cpf1 polypeptide has no substantial nuclease activity.

In some cases, the chimeric site-directed modifying polypeptide comprises an amino acid sequence having at least about 75%, at least about 80%, at least about 85%, at least about 90%, at least about 95%, at least about 99% or 100% amino acid sequence identity to FIG. 1, or to the corresponding portions in any of the amino acid sequences set forth in FIG. 1. In some embodiments, the activity portion of the site-directed modifying polypeptide comprises a heterologous polypeptide that has DNA-modifying activity and/or transcription factor activity and/or DNA-associated polypeptide-modifying activity. In some cases, a heterologous polypeptide replaces a portion of the Cpf1 polypeptide that provides nuclease activity. In other embodiments, a site-directed modifying polypeptide comprises both a portion of the Cpf1 polypeptide that normally provides nuclease activity (and that portion can be fully active or can instead be modified to have less than 100% of the corresponding wild-type activity) and a heterologous polypeptide. In other words, in some cases, a chimeric site-directed modifying polypeptide is a fusion polypeptide comprising both the portion of the Cpf1 polypeptide that normally provides nuclease activity and the heterologous polypeptide. In other cases, a chimeric site-directed modifying polypeptide is a fusion polypeptide comprising a modified variant of the activity portion of the Cpf1 polypeptide (e.g., amino acid change, deletion, insertion) and a heterologous polypeptide. In yet other cases, a chimeric site-directed modifying polypeptide is a fusion polypeptide comprising a heterologous polypeptide and the RNA-binding portion of a naturally occurring or a modified site-directed modifying polypeptide.

For example, in a chimeric Cpf1 protein, a naturally occurring (or modified, e.g., mutation, deletion, insertion) Cpf1 polypeptide may be fused to a heterologous polypeptide sequence (i.e., a polypeptide sequence from a protein other than Cpf1 or a polypeptide sequence from another organism). The heterologous polypeptide sequence may exhibit an activity (e.g., enzymatic activity) that will also be exhibited by the chimeric Cpf1 protein (e.g., methyltransferase activity, acetyltransferase activity, kinase activity, ubiquitinating activity, etc.). A heterologous nucleic acid sequence may be linked to another nucleic acid sequence (e.g., by genetic engineering) to generate a chimeric nucleotide sequence encoding a chimeric polypeptide. In some embodiments, a chimeric Cpf1 polypeptide is generated by fusing a Cpf1 polypeptide (e.g., wild type Cpf1 or a Cpf1 variant, e.g., a Cpf1 with reduced or inactivated nuclease activity) with a heterologous sequence that provides for subcellular localization (e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, the heterologous sequence can provide a tag for ease of tracking or purification (e.g., a fluorescent protein, e.g., green fluorescent protein (GFP), YFP, RFP, CFP, mCherry, tdTomato, and the like; a HIS tag, e.g., a 6×His tag; a hemagglutinin (HA) tag; a FLAG tag; a Myc tag; and the like). In some embodiments, the heterologous sequence can provide for increased or decreased stability. In some embodiments, the heterologous sequence can provide a binding domain (e.g., to provide the ability of a chimeric Cpf1 polypeptide to bind to another protein of interest, e.g., a DNA or histone modifying protein, a transcription factor or transcription repressor, a recruiting protein, etc.).

Nucleic Acid Encoding a Chimeric Site-Directed Modifying Polypeptide

The present disclosure provides a nucleic acid comprising a nucleotide sequence encoding a chimeric site-directed modifying polypeptide. In some embodiments, the nucleic acid comprising a nucleotide sequence encoding a chimeric site-directed modifying polypeptide is an expression vector, e.g., a recombinant expression vector.

In some embodiments, a method involves contacting a target DNA or introducing into a cell (or a population of cells) one or more nucleic acids comprising a chimeric site-directed modifying polypeptide. Suitable nucleic acids comprising nucleotide sequences encoding a chimeric site-directed modifying polypeptide include expression vectors, where an expression vector comprising a nucleotide sequence encoding a chimeric site-directed modifying polypeptide is a “recombinant expression vector.”

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding a chimeric site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a chimeric site-directed modifying polypeptide is operably linked to multiple control elements that allow expression of the nucleotide sequence encoding a chimeric site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-l. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin (HA) tag, a fluorescent protein (e.g., a green fluorescent protein; a yellow fluorescent protein, etc.), etc.) that are fused to the chimeric site-directed modifying polypeptide.

In some embodiments, a nucleotide sequence encoding a chimeric site-directed modifying polypeptide is operably linked to an inducible promoter (e.g., heat shock promoter, Tetracycline-regulated promoter, Steroid-regulated promoter, Metal-regulated promoter, estrogen receptor-regulated promoter, etc.). In some embodiments, a nucleotide sequence encoding a chimeric site-directed modifying polypeptide is operably linked to a spatially restricted and/or temporally restricted promoter (e.g., a tissue specific promoter, a cell type specific promoter, etc.). In some embodiments, a nucleotide sequence encoding a chimeric site-directed modifying polypeptide is operably linked to a constitutive promoter.

Methods of introducing a nucleic acid into a host cell are known in the art, and any known method can be used to introduce a nucleic acid (e.g., an expression construct) into a stem cell or progenitor cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

Methods

The present disclosure provides methods for modifying a target DNA and/or a target DNA-associated polypeptide. Generally, a method involves contacting a target DNA with a complex (a “targeting complex”), which complex comprises a guide RNA and a site-directed modifying polypeptide.

As discussed above, a guide RNA and a site-directed modifying polypeptide form a complex. The guide RNA provides target specificity to the complex by comprising a nucleotide sequence that is complementary to a sequence of a target DNA. The site-directed modifying polypeptide of the complex provides the site-specific activity. In some embodiments, a complex modifies a target DNA, leading to, for example, DNA cleavage, DNA methylation, DNA damage, DNA repair, etc. In other embodiments, a complex modifies a target polypeptide associated with target DNA (e.g., a histone, a DNA-binding protein, etc.), leading to, for example, histone methylation, histone acetylation, histone ubiquitination, and the like. The target DNA may be, for example, naked DNA in vitro, chromosomal DNA in cells in vitro, chromosomal DNA in cells in vivo, etc.

In some cases, different Cpf1 proteins (i.e., Cpf1 proteins from various species) may be advantageous to use in the various provided methods in order to capitalize on various enzymatic characteristics of the different Cpf1 proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.). The method of processing guide crRNA, wherein the method comprises contacting a longer form crRNA with a Cpf1 polypeptide under conditions that allow Cpf1 to cleave the guide crRNA into smaller fragments, at least one of which is capable of directing Cpf1 to a target site, said method being performed in the absence of Cas9 or tracrRNA.

Cpf1 proteins from various species may require different PAM sequences in the target DNA. Thus, for a particular Cpf1 protein of choice, the PAM sequence requirement may be different than the PAM sequence described above.

Exemplary methods provided that take advantage of characteristics of Cpf1 orthologs include the following.

The nuclease activity cleaves target DNA to produce double strand breaks. These breaks are then repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor polynucleotide. In some cases, a donor polynucleotide is introduced into a cell. The modifications of the target DNA due to NHEJ and/or homology-directed repair lead to, for example, gene correction, gene replacement, gene tagging, transgene insertion, nucleotide deletion, gene disruption, gene mutation, sequence replacement, etc. Accordingly, cleavage of DNA by a site-directed modifying polypeptide may be used to delete nucleic acid material from a target DNA sequence (e.g., to disrupt a gene that makes cells susceptible to infection (e.g., the CCRS or CXCR4 gene, which makes T cells susceptible to HIV infection, to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material into a locus of choice in the target DNA.

Alternatively, if a guide RNA and a site-directed modifying polypeptide are coadministered to cells with a donor polynucleotide sequence that includes at least a segment with homology to the target DNA sequence, the subject methods may be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a guide RNA and a site-directed modifying polypeptide is useful in any in vitro or in vivo application in which it is desirable to modify DNA in a site-specific, i.e., “targeted”, way, for example gene knock-out, gene knock-in, gene editing, gene tagging, sequence replacement, etc., as used in, for example, gene therapy, e.g., to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

In some embodiments, the site-directed modifying polypeptide comprises a modified form of the Cpf1 protein. In some instances, the modified form of the Cpf1 protein comprises an amino acid change (e.g., deletion, insertion, or substitution) that reduces the naturally occurring nuclease activity of the Cpf1 protein. For example, in some instances, the modified form of the Cpf1 protein has less than 50%, less than 40%, less than 30%, less than 20%, less than 10%, less than 5%, or less than 1% of the nuclease activity of the corresponding wild-type Cpf1 polypeptide. In some cases, the modified form of the Cpf1 polypeptide has no substantial nuclease activity. When a site-directed modifying polypeptide is a modified form of the Cpf1 polypeptide that has no substantial nuclease activity, it can be referred to as “dCpf1.”

In some embodiments, the site-directed modifying polypeptide comprises a heterologous sequence (e.g., a fusion). In some embodiments, a heterologous sequence can provide for subcellular localization of the site-directed modifying polypeptide (e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, a heterologous sequence can provide a tag for ease of tracking or purification (e.g., a fluorescent protein, e.g., green fluorescent protein (GFP), YFP, RFP, CFP, mCherry, tdTomato, and the like; a his tag, e.g., a 6×His tag; a hemagglutinin (HA) tag; a FLAG tag; a Myc tag; and the like). In some embodiments, the heterologous sequence can provide for increased or decreased stability.

In some embodiments, a site-directed modifying polypeptide can be codon-optimized. This type of optimization is known in the art and entails the mutation of foreign-derived DNA to mimic the codon preferences of the intended host organism or cell while encoding the same protein. Thus, the codons are changed, but the encoded protein remains unchanged. For example, if the intended target cell were a human cell, a human codon-optimized Cpf1 (or variant, e.g., enzymatically inactive variant) would be a suitable site-directed modifying polypeptide. Any suitable site-directed modifying polypeptide (e.g., any Cpf1 such as the sequence set forth in FIG. 1) can be codon optimized. As another non-limiting example, if the intended host cell were a mouse cell, than a mouse codon-optimized Cpf1 (or variant, e.g., enzymatically inactive variant) would be a suitable site-directed modifying polypeptide. While codon optimization is not required, it is acceptable and may be preferable in certain cases.

In some embodiments, a guide RNA and a site-directed modifying polypeptide are used as an inducible system for shutting off gene expression in bacterial cells. In some cases, nucleic acids encoding an appropriate guide RNA and/or an appropriate site-directed polypeptide are incorporated into the chromosome of a target cell and are under control of an inducible promoter. When the guide RNA and/or the site-directed polypeptide are induced, the target DNA is cleaved (or otherwise modified) at the location of interest (e.g., a target gene on a separate plasmid), when both the guide RNA and the site-directed modifying polypeptide are present and form a complex. As such, in some cases, bacterial expression strains are engineered to include nucleic acid sequences encoding an appropriate site-directed modifying polypeptide in the bacterial genome and/or an appropriate guide RNA on a plasmid (e.g., under control of an inducible promoter), allowing experiments in which the expression of any targeted gene (expressed from a separate plasmid introduced into the strain) could be controlled by inducing expression of the guide RNA and the site-directed polypeptide.

In some cases, the site-directed modifying polypeptide has enzymatic activity that modifies target DNA in ways other than introducing double strand breaks. Enzymatic activity of interest that may be used to modify target DNA (e.g., by fusing a heterologous polypeptide with enzymatic activity to a site-directed modifying polypeptide, thereby generating a chimeric site-directed modifying polypeptide) includes, but is not limited methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). Methylation and demethylation is recognized in the art as an important mode of epigenetic gene regulation while DNA damage and repair activity is essential for cell survival and for proper genome maintenance in response to environmental stresses.

As such, the methods herein find use in the epigenetic modification of target DNA and may be employed to control epigenetic modification of target DNA at any location in a target DNA by genetically engineering the desired complementary nucleic acid sequence into the DNA-targeting segment of a guide RNA. The methods herein also find use in the intentional and controlled damage of DNA at any desired location within the target DNA. The methods herein also find use in the sequence-specific and controlled repair of DNA at any desired location within the target DNA. Methods to target DNA-modifying enzymatic activities to specific locations in target DNA find use in both research and clinical applications.

In some cases, the site-directed modifying polypeptide has activity that modulates the transcription of target DNA (e.g., in the case of a chimeric site-directed modifying polypeptide, etc.). In some cases, a chimeric site-directed modifying polypeptides comprising a heterologous polypeptide that exhibits the ability to increase or decrease transcription (e.g., transcriptional activator or transcription repressor polypeptides) is used to increase or decrease the transcription of target DNA at a specific location in a target DNA, which is guided by the DNA-targeting segment of the guide RNA. Examples of source polypeptides for providing a chimeric site-directed modifying polypeptide with transcription modulatory activity include, but are not limited to light-inducible transcription regulators, small molecule/drug-responsive transcription regulators, transcription factors, transcription repressors, etc. In some cases, the method is used to control the expression of a targeted coding-RNA (protein-encoding gene) and/or a targeted non-coding RNA (e.g., tRNA, rRNA, snoRNA, siRNA, miRNA, long ncRNA, etc.). In some cases, the site-directed modifying polypeptide has enzymatic activity that modifies a polypeptide associated with DNA (e.g., histone). In some embodiments, the enzymatic activity is methyltransferase activity, demethylase activity, acetyltransferase activity, deacetylase activity, kinase activity, phosphatase activity, ubiquitin ligase activity (i.e., ubiquitination activity), deubiquitinating activity, adenylation activity, deadenylation activity, SUMOylating activity, deSUMOylating activity, ribosylation activity, deribosylation activity, myristoylation activity, demyristoylation activity glycosylation activity (e.g., from GIcNAc transferase) or deglycosylation activity. The enzymatic activities listed herein catalyze covalent modifications to proteins. Such modifications are known in the art to alter the stability or activity of the target protein (e.g., phosphorylation due to kinase activity can stimulate or silence protein activity depending on the target protein). Of particular interest as protein targets are histones. Histone proteins are known in the art to bind DNA and form complexes known as nucleosomes. Histones can be modified (e.g., by methylation, acetylation, ubiquitination, phosphorylation) to elicit structural changes in the surrounding DNA, thus controlling the accessibility of potentially large portions of DNA to interacting factors such as transcription factors, polymerases and the like. A single histone can be modified in many different ways and in many different combinations (e.g., trimethylation of lysine 27 of histone 3, H3K27, is associated with DNA regions of repressed transcription while trimethylation of lysine 4 of histone 3, H3K4, is associated with DNA regions of active transcription). Thus, a site-directed modifying polypeptide with histone-modifying activity finds use in the site specific control of DNA structure and can be used to alter the histone modification pattern in a selected region of target DNA. Such methods find use in both research and clinical applications.

In some embodiments, multiple guide RNAs are used simultaneously to simultaneously modify different locations on the same target DNA or on different target DNAs. In some embodiments, two or more guide RNAs target the same gene or transcript or locus. In some embodiments, two or more guide RNAs target different unrelated loci. In some embodiments, two or more guide RNAs target different, but related loci.

In some cases, the site-directed modifying polypeptide is provided directly as a protein. As one non-limiting example, fungi (e.g., yeast) can be transformed with exogenous protein and/or nucleic acid using spheroplast transformation (see Kawai et al., Bioeng Bugs. 2010 November-December; 1(6):395-403: “Transformation of Saccharomyces cerevisiae and other fungi: methods and possible underlying mechanism”; and Tanka et al., Nature. 2004 Mar. 18; 428(6980):323-8: “Conformational variations in an infectious protein determine prion strain differences”; both of which are herein incorporated by reference in their entirety). Thus, a site-directed modifying polypeptide (e.g., Cpf1) can be incorporated into a spheroplast (with or without nucleic acid encoding a guide RNA and with or without a donor polynucleotide) and the spheroplast can be used to introduce the content into a yeast cell. A site-directed modifying polypeptide can be introduced into a cell (provided to the cell) by any convenient method; such methods are known to those of ordinary skill in the art. As another non-limiting example, a site-directed modifying polypeptide can be injected directly into a cell (e.g., with or without nucleic acid encoding a guide RNA and with or without a donor polynucleotide), e.g., a cell of a zebrafish embryo, the pronucleus of a fertilized mouse oocyte, etc.

Target Cells of Interest

In some of the above applications, the methods may be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual). Because the guide RNA provide specificity by hybridizing to target DNA, a mitotic and/or post-mitotic cell of interest in the disclosed methods may include a cell from any organism (e.g., a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal, a cell from a rodent, a cell from a primate, a cell from a human, etc.).

Any type of cell may be of interest (e.g., a stem cell, e.g., an embryonic stem (ES) cell, an induced pluripotent stem (iPS) cell, a germ cell; a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. Target cells are in many embodiments unicellular organisms, or are grown in culture.

If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

Nucleic Acids Encoding a Guide RNA and/or a Site-Directed Modifying Polypeptide

In some embodiments, a method involves contacting a target DNA or introducing into a cell (or a population of cells) one or more nucleic acids comprising nucleotide sequences encoding a guide RNA and/or a site-directed modifying polypeptide and/or a donor polynucleotide. Suitable nucleic acids comprising nucleotide sequences encoding a guide RNA and/or a site-directed modifying polypeptide include expression vectors, where an expression vector comprising a nucleotide sequence encoding a guide RNA and/or a site-directed modifying polypeptide is a “recombinant expression vector.”

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

In some embodiments, a nucleotide sequence encoding a guide RNA and/or a site-directed modifying polypeptide is operably linked to a control element, e.g., a transcriptional control element, such as a promoter. The transcriptional control element may be functional in either a eukaryotic cell, e.g., a mammalian cell, or a prokaryotic cell (e.g., bacterial or archaeal cell). In some embodiments, a nucleotide sequence encoding a guide RNA and/or a site-directed modifying polypeptide is operably linked to multiple control elements that allow expression of the nucleotide sequence encoding a guide RNA and/or a site-directed modifying polypeptide in both prokaryotic and eukaryotic cells.

Depending on the host/vector system utilized, any of a number of suitable transcription and translation control elements, including constitutive and inducible promoters, transcription enhancer elements, transcription terminators, etc. may be used in the expression vector (e.g., U6 promoter, H1 promoter, etc.; see above) (see e.g., Bitter et al. (1987) Methods in Enzymology, 153:516-544).

In some embodiments, a guide RNA and/or a site-directed modifying polypeptide can be provided as RNA. In such cases, the guide RNA and/or the RNA encoding the site-directed modifying polypeptide can be produced by direct chemical synthesis or may be transcribed in vitro from a DNA encoding the guide RNA. Methods of synthesizing RNA from a DNA template are well known in the art. In some cases, the guide RNA and/or the RNA encoding the site-directed modifying polypeptide will be synthesized in vitro using an RNA polymerase enzyme (e.g., T7 polymerase, T3 polymerase, SP6 polymerase, etc.). Once synthesized, the RNA may directly contact a target DNA or may be introduced into a cell by any of the well-known techniques for introducing nucleic acids into cells (e.g., microinjection, electroporation, transfection, etc.).

Nucleotides encoding a guide RNA (introduced either as DNA or RNA) and/or a site-directed modifying polypeptide (introduced as DNA or RNA) and/or a donor polynucleotide may be provided to the cells using well-developed transfection techniques; see, e.g., Angel and Yanik (2010) PLoS ONE 5(7): e 11756, and the commercially available TransMessenger@ reagents from Qiagen, Stemfect™ RNA Transfection Kit from Stemgent, and TranslT®-mRNA Transfection Kit from Mims Bio. See also Beumer et al. (2008) Efficient gene targeting in Drosophila by direct embryo injection with zinc-finger nucleases. PNAS 105(50):19821-19826. Alternatively, nucleic acids encoding a guide RNA and/or a site-directed modifying polypeptide and/or a chimeric site-directed modifying polypeptide and/or a donor polynucleotide may be provided on DNA vectors. Many vectors, e.g., plasmids, cosmids, minicircles, phage, viruses, etc., useful for transferring nucleic acids into target cells are available. The vectors comprising the nucleic acid(s) may be maintained episomally, e.g., as plasmids, minicircle DNAs, viruses such cytomegalovirus, adenovirus, etc., or they may be integrated into the target cell genome, through homologous recombination or random integration, e.g., retrovirus-derived vectors such as MMLV, HIV-1, ALV, etc.

Vectors may be provided directly to the cells. In other words, the cells are contacted with vectors comprising the nucleic acid encoding guide RNA and/or a site-directed modifying polypeptide and/or a chimeric site-directed modifying polypeptide and/or a donor polynucleotide such that the vectors are taken up by the cells. Methods for contacting cells with nucleic acid vectors that are plasmids, including electroporation, calcium chloride transfection, microinjection, and lipofection are well known in the art. For viral vector delivery, the cells are contacted with viral particles comprising the nucleic acid encoding a guide RNA and/or a site-directed modifying polypeptide and/or a chimeric site-directed modifying polypeptide and/or a donor polynucleotide. Retroviruses, for example, lentiviruses, are particularly suitable to the method of the invention. Commonly used retroviral vectors are “defective”, i.e., unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid encoding the reprogramming factors into packaging cell lines and of collecting the viral particles that are generated by the packaging lines are well known in the art. Nucleic acids can also be introduced by direct micro-injection (e.g., injection of RNA into a zebrafish embryo).

Vectors used for providing the nucleic acids encoding guide RNA and/or a site-directed modifying polypeptide and/or a chimeric site-directed modifying polypeptide and/or a donor polynucleotide to the cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CMV-13-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing a guide RNA and/or a site-directed modifying polypeptide and/or a chimeric site-directed modifying polypeptide and/or a donor polynucleotide to the cells may include nucleic acid sequences that encode for selectable markers in the target cells, so as to identify cells that have taken up the guide RNA and/or a site-directed modifying polypeptide and/or a chimeric site-directed modifying polypeptide and/or a donor polynucleotide.

A guide RNA and/or a site-directed modifying polypeptide and/or a chimeric site-directed modifying polypeptide may instead be used to contact DNA or introduced into cells as RNA. Methods of introducing RNA into cells are known in the art and may include, for example, direct injection, transfection, or any other method used for the introduction of DNA. A site-directed modifying polypeptide may instead be provided to cells as a polypeptide. Such a polypeptide may optionally be fused to a polypeptide domain that increases solubility of the product. The domain may be linked to the polypeptide through a defined protease cleavage site, e.g., a TEV sequence, which is cleaved by TEV protease. The linker may also include one or more flexible sequences, e.g., from 1 to 10 glycine residues. In some embodiments, the cleavage of the fusion protein is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally or alternatively, the site-directed modifying polypeptide may be fused to a polypeptide permeant domain to promote uptake by the cell. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO:50). As another example, the permeant peptide comprises the HIV-1 tat basic region amino acid sequence, which may include, for example, amino acids 49-57 of naturally occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A. 2000 Nov. 21; 97(24):13003-8; published US Patent Application Publications Nos. 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

A site-directed modifying polypeptide may be produced in vitro or by eukaryotic cells or by prokaryotic cells, and it may be further processed by unfolding, e.g., heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine.

Also included in the invention are guide RNAs and site-directed modifying polypeptides that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable as a therapeutic agent. Analogs of such polypeptides include those containing residues other than naturally occurring L-amino acids, e.g., D-amino acids or non-naturally occurring synthetic amino acids. D-amino acids may be substituted for some or all of the amino acid residues. The site-directed modifying polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The site-directed modifying polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. To induce DNA cleavage and recombination, or any desired modification to a target DNA, or any desired modification to a polypeptide associated with target DNA, the guide RNA and/or the site-directed modifying polypeptide and/or the donor polynucleotide, whether they be introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the cells one or more times, e.g., one time, twice, three times, or more than three times, and the cells allowed to incubate with the agent(s) for some amount of time following each contacting event e.g., 16-24 hours, after which time the media is replaced with fresh media and the cells are cultured further. In cases in which two or more different targeting complexes are provided to the cell (e.g., two different guide RNAs that are complementary to different sequences within the same or different target DNA), the complexes may be provided simultaneously (e.g., as two polypeptides and/or nucleic acids), or delivered simultaneously. Alternatively, they may be provided consecutively, e.g., the targeting complex being provided first, followed by the second targeting complex, etc. or vice versa.

Typically, an effective amount of the guide RNA and/or site-directed modifying polypeptide and/or donor polynucleotide is provided to the target DNA or cells to induce target modification. An effective amount of the guide RNA and/or site-directed modifying polypeptide and/or donor polynucleotide is the amount to induce a 2-fold increase or more in the amount of target modification observed between two homologous sequences relative to a negative control, e.g., a cell contacted with an empty vector or irrelevant polypeptide. That is to say, an effective amount or dose of the guide RNA and/or site-directed modifying polypeptide and/or donor polynucleotide will induce a 2-fold increase, a 3-fold increase, a 4-fold increase or more in the amount of target modification observed at a target DNA region, in some instances a 5-fold increase, a 6-fold increase or more, sometimes a 7-fold or 8-fold increase or more in the amount of recombination observed, e.g., an increase of 10-fold, 50-fold, or 100-fold or more, in some instances, an increase of 200-fold, 500-fold, 700-fold, or 1000-fold or more, e.g., a 5000-fold, or 10,000-fold increase in the amount of recombination observed. The amount of target modification may be measured by any convenient method. For example, a silent reporter construct comprising complementary sequence to the targeting segment (targeting sequence) of the guide RNA flanked by repeat sequences that, when recombined, will reconstitute a nucleic acid encoding an active reporter may be cotransfected into the cells, and the amount of reporter protein assessed after contact with the guide RNA and/or site-directed modifying polypeptide and/or donor polynucleotide, e.g., 2 hours, 4 hours, 8 hours, 12 hours, 24 hours, 36 hours, 48 hours, 72 hours or more after contact with the guide RNA and/or site-directed modifying polypeptide and/or donor polynucleotide. As another, more sensitivity assay, for example, the extent of recombination at a genomic DNA region of interest comprising target DNA sequences may be assessed by PCR or Southern hybridization of the region after contact with a guide RNA and/or site-directed modifying polypeptide and/or donor polynucleotide, e.g., 2 hours, 4 hours, 8 hours, 12 hours, 24 hours, 36 hours, 48 hours, 72 hours or more after contact with the guide RNA and/or site-directed modifying polypeptide and/or donor polynucleotide.

Contacting the cells with a guide RNA and/or site-directed modifying polypeptide and/or donor polynucleotide may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g., penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are typically permissive of nonhomologous end joining and homology-directed repair. In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide. The donor sequence may comprise certain sequence differences as compared to the genomic sequence, e.g., restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and 0-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a guide RNA and/or site-directed modifying polypeptide and/or donor polynucleotide.

Following the methods described above, a DNA region of interest may be cleaved and modified, i.e., “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g., magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e., grown under conditions that promote their proliferation. Culture medium may be liquid or semi-solid, e.g., containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g., penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g., to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g., mouse, rat, guinea pig, hamster, lagomorpha (e.g., rabbit), etc.) may be used for experimental investigations.

Cells may be provided to the subject alone or with a suitable substrate or matrix, e.g., to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the site of injury, include, e.g., through an Ommaya reservoir, e.g., for intrathecal delivery (see e.g., U.S. Pat. Nos. 5,222,982 and 5,385,582, incorporated herein by reference); by bolus injection, e.g., by a syringe, e.g., into a joint; by continuous infusion, e.g., by cannulation, e.g., with convection (see e.g., US Application No. 20070254842, incorporated herein by reference); or by implanting a device upon which the cells have been reversibly affixed (see e.g., US Application Nos. 20080081064 and 20090196903, incorporated herein by reference). Cells may also be introduced into an embryo (e.g., a blastocyst) for the purpose of generating a transgenic animal (e.g., a transgenic mouse).

Multiplex Gene Editing

The well-studied Types I, II and Ill CRISPR-Cas systems involve a set of distinct Cas proteins for production of mature crRNAs and interference with invading nucleic acids. In Types I and III, Cas6 or Cas5d cleave pre-crRNA. The matured crRNAs then guide a complex of Cas proteins (Cascade-Cas3, Type I; Csm or Cmr, Type III) to target and cleave invading DNA or RNA. In Type II, RNase III cleaves pre-crRNA base-paired with tracrRNA in the presence of Cas9. The mature tracrRNA:crRNA duplex guides Cas9 to cleave target DNA.

On the other hand, Type V-A Cpf1 is a dual-nuclease in crRNA biogenesis and interference. Cpf1 cleaves pre-crRNA 4 nt upstream of a hairpin structure formed within the repeats to generate intermediate crRNAs. Cpf1 guided by mature repeat-spacer crRNAs introduces double-stranded breaks in target DNA. Thus, Cpf1 is therefore an ideal protein to perform multiplexing because it processes the RNA and cleaves the DNA.

Multiplexing means editing the DNA multiple times in multiple locations.

For multiplexing with Cas9, for example, one needs multiple guide RNAs provided exogenously or expressed independently, endogenously within the cell or system. However, for Cpf1, one only needs one Cpf1 enzyme and one repeat-spacer array under the control of one promoter. Cpf1 then cleaves the pre-crRNA to produce the single guide RNAs that can then target Cpf1 to the genome. One advantage of the present described system is that all the crRNAs also called guide RNAs are present in the same cell, which greatly increases the proportion of cells in which many, most or all of the intended multiplex editing occurs, and greatly decreases the proportion of cells in which only one or a limited number of the intended multiplex editing events occur. Furthermore, the location and structure of the crRNAs elements within the pre-crRNA will impact the endonuclease activity of Cpf1. Consequently, it is contemplated here that structure, whether repeat-spacer or spacer-repeat, length or location of repeats, nature of the stem-loop, chemical modifications to, intervening sequences or chemical structures between, or order of crRNA sequences in a heterologous pre-crRNA molecule, or other factors can be modulated or manipulated to modify the endonuclease activity at each of the sites specified by crRNA spacer sequences in the heterologuous pre-crRNA.

Additional aspects of the invention derive from multiplex editing in the context of a Cpf1 or other type V-A endonuclease that cleaves double stranded DNA in a manner that leaves a 5′ overhang at the cleaved ends. Each cleavage site is directed by a unique gRNA sequence. pre-Consequently the resultant 5′ overhang is a sequence of 5 nucleotides that is relatively unique and specific to the particular gRNA or crRNA specifying the cleavage site. The relative uniqueness of the 5′ overhang is expected to be 4e5, or occurring once every 1024 cleavage sites (assuming random variation in nucleotides in the genome). In a setting where more than one gRNA (or more than one gRNA sequence in a crRNA) is employed for multiplex editing, the resultant 5′ overhang sequences will be more likely to re-anneal with the intended partner cleavage sites, rather than with a heterologous end, as would occur in the formation of chromosomal translocations. Consequently the use of Cpf1 may be a preferred method for multiplex genome editing to improve gene disruption at multiple loci and reduce the occurrence of chromosomal translocations during multiplex editing. It is understood that certain cell types may harbor endogenous single strand DNA exonuclease activity, such that a 5′ single strand DNA overhang may be partially or fully cleaved resulting in no 5′ overhang or a partial 5′ overhang. It is anticipated that this system of single strand DNA exonuclease activity or other cellular systems that regulate the presence or activity of non-homologous 5′ overhang sequences, may have kinetic or physiologic characteristics that can be manipulated or exploited, for example by physiologic or pharmacologic or other means, to reduce the likelihood of heterologous end joining and resultant chromosomal translocations.

A non-limiting example of a multiplexing method is a method for editing a gene at multiple locations in a cell consisting essentially of: i) introducing a Cpf1 polypeptide or a nucleic acid encoding a Cpf1 polypeptide into the cell; and ii) introducing a single heterologous nucleic acid comprising one or more pre-crRNAs either as RNA or encoded as DNA under the control of one promoter into the cell, each pre-crRNA comprising a repeat-spacer array, wherein the spacer comprises a nucleic acid sequence that is complementary to a target sequence in the DNA and the repeat comprises a stem-loop structure, wherein the Cpf1 polypeptide cleaves the pre-crRNA(s) upstream of the stem-loop structure to generate two or more intermediate crRNAs, wherein the two or more intermediate crRNAs are processed into two or more mature crRNAs, and wherein each two or more mature crRNAs guides the Cpf1 polypeptide to effect two or more double-strand breaks (DSBs) into the DNA. For example, the method may further comprise introducing into the cell one or more polynucleotide donor templates. The one or more polynucleotide donor templates may be linked to the pre-crRNA. The DNA is repaired at each of the two or more DSBs by either homology directed repair, non-homologous end joining, or microhomology-mediated end joining, or other biological process. The DNA is corrected at each of the two or more DSBs by either deletion, insertion, or replacement of the DNA. Alternatively, if a DNase-deficient Cpf1 polypeptide fused to a dimeric FOK1 nuclease or other biologically active moiety or moieties are employed to so affect a biological process in a site specific manner, the modified Cpf1 polypeptide can be directed to the specific sites in the DNA by co-administration of a single heterologous pre-crRNA, or a single heterologous nucleic acid under the control of one promoter.

An example of a multiplexing composition is a composition for editing a gene at multiple locations in a cell consisting essentially of: i) a Cpf1 polypeptide or a nucleic acid encoding a Cpf1 polypeptide; and ii) a single heterologous nucleic acid comprising pre-crRNA under the control of one promoter into the cell, pre-crRNA comprising a repeat-spacer array, wherein the spacer comprises a nucleic acid sequence that is complementary to a target sequence in the DNA and the repeat comprises a stem-loop structure. The composition may further comprise one or more polynucleotide donor templates. The one or more polynucleotide donor templates may be linked to the pre-crRNA.

An additional aspect of the present invention derives from multiplex editing in the context of a Cpf1 or other type V-A endonuclease that cleaves double stranded DNA in a manner that leaves a 5′ overhang at the cleaved ends. Each cleavage site is directed by a unique gRNA sequence or a unique sequence within the CRISPR array (pre crRNA) that is subsequently processed into gRNA by Cpf1. Consequently, the resultant 5′ overhang is a sequence of 5 nucleotides that is relatively unique and specific to the particular gRNA or crRNA specifying the cleavage site. The relative uniqueness of the 5′ overhang is expected to be 4 to the power of 5, or in other words, occurring once every 1024 cleavage sites (assuming random variation in nucleotides in the genome). In a setting where more than one gRNA (or more than one gRNA sequence in a crRNA) is employed for multiplex editing, the resultant 5′ overhang sequences will be more likely to reanneal with the original partner cleavage sites, rather than with a heterologous end as would occur in the formation of chromosomal translocations. Consequently the use of Cpf1 may be a preferred method for multiplex genome editing to improve gene disruption at multiple loci and reduce the occurrence of chromosomal translocations during multiplex editing. It is understood that certain cell types may harbor endogenous single strand DNA exonuclease activity, such that a 5′ single strand DNA overhang may be partially or fully cleaved resulting in no 5′ overhang or a partial 5′ overhang. It is anticipated that this system of single strand DNA exonuclease activity or other cellular systems that regulate the presence or activity of non-homologous 5′ overhang sequences, may have kinetic or physiologic characteristics that can be manipulated or exploited, for example by physiologic or pharmacologic or other means, to reduce the likelihood of heterologous end joining and resultant chromosomal translocations.

Additional Methods

The invention includes a method for processing pre-crRNA into mature crRNA by a Cpf1 polypeptide in a manner that renders the mature crRNA available for directing the Cpf1 DNA endonuclease activity. In some embodiments of the method, the Cpf1 polypeptide is more readily complexed with the mature crRNA, and thus more readily available for directing DNA endonuclease activity as a consequence of this crRNA being processed by the same Cpf1 polypeptide from the pre-crRNA. In some embodiments of the method, the Cpf1 polypeptide is able to cleave, isolate or purify one or more mature crRNAs from a modified pre-crRNA oligonucleotide sequence in which heterologous sequences are incorporated 5′ or 3′ to one or more crRNA sequences within a RNA oligonucleotide or DNA expression construct. In some embodiments of the method, the heterologous sequences can be incorporated to modify the stability, half life, expression level or timing, interaction with the Cpf1 polypeptide or target DNA sequence, or any other physical or biochemical characteristics known in the art. In some embodiments of the method, the pre-crRNA sequence is modified to provide for differential regulation of two or more mature crRNA sequences within the pre-crRNA sequence, to differentially modify the stability, half life, expression level or timing, interaction with the Cpf1 polypeptide or target DNA sequence, or any other physical or biochemical characteristics.

The invention also includes a method for targeting, editing or manipulating DNA in a cell comprising linking an intact or partially or fully deficient Cpf1 polypeptide or pre-crRNA or crRNA moiety, to a dimeric FOK1 nuclease to direct endonuclease cleavage, as directed to one or more specific DNA target sites by one or more crRNA molecules. In some embodiments, the Cpf1 polypeptide linked with a dimeric FOK1 nuclease is introduced into the cell together with a heterologous pre-crRNAs either as RNA or encoded as DNA and under the control of one promoter into the cell, pre-crRNA comprising a repeat-spacer array, wherein the spacer comprises a nucleic acid sequence that is complementary to a target sequence in the DNA and the repeat comprises a stem-loop structure, wherein the Cpf1 polypeptide cleaves the pre-crRNAs upstream of the stem-loop structures of the repeat to generate two or more intermediate crRNAs.

The invention includes a method for targeting, editing or manipulating DNA in a cell comprising linking an intact or partially or fully deficient Cpf1 polypeptide or pre-crRNA or crRNA moiety, to a donor single or double strand DNA donor template to facilitate homologous recombination of exogenous DNA sequences, as directed to one or more specific DNA target sites by one or more crRNA molecules.

Also, the invention includes a method for targeting, editing or manipulating DNA in a cell comprising linking an intact or partially or fully deficient Cpf1 polypeptide or pre-crRNA or crRNA moiety, to a transcriptional activator or repressor, or epigenetic modifier such as a methylase, demethylase, acetylase, or deacetylase, or signaling or detection, to facilitate the modulation of expression or signaling, detection or activation, as directed to one or more specific DNA target sites by one or more crRNA molecules.

The invention includes a method for directing a polynucleotide donor template to the specific site of gene editing comprising linking the polynucleotide donor template to a crRNA or a guide RNA. In some embodiments, the polynucleotide donor template is single stranded. In some embodiments, the polynucleotide donor template is double stranded. The polynucleotide donor template may be linked to a crRNA or a guide RNA by any means known in the art, such as an ionic bond, a covalent bond, or a chemical linker. In some embodiments, the polynucleotide donor template remains linked to the crRNA or within the guide RNA. In some embodiments, Cpf1 cleaves the pre-crRNA, or guide RNA, thus liberating the polynucleotide donor template to facilitate homology directed repair. The invention also includes a composition comprising a polynucleotide donor template linked to a crRNA or a guide RNA.

The invention also includes a method for targeting, editing or manipulating DNA in a cell comprising linking a pre-crRNA or crRNA or guide RNA to a donor single or double strand polynucleotide donor template such that the donor template is cleaved from the pre-crRNA or crRNA or guide RNA by a Cpf1 polypeptide, thus facilitating homology directed repair by the donor template, as directed to one or more specific DNA target sites by one or more guide RNA or crRNA molecules

Guide RNA polynucleotides (RNA or DNA) and/or Cpf1 polynucleotides (RNA or DNA) can be delivered by viral or non-viral delivery vehicles known in the art.

Polynucleotides may be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

A recombinant adeno-associated virus (AAV) vector may be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, WO 01/83692.

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. No. 5,786,211; U.S. Pat. No. 5,871,982; and U.S. Pat. No. 6,258,595.

AAV vector serotypes can be matched to target cell types. For example, the following exemplary cell types transduced by the indicated AAV serotypes among others.

| Tissue/Cell Type | Serotype |
|---|---|
| Liver | AAV8, AAV9 |
| Skeletal muscle | AAV1, AAV7, AAV6, AAV8, AAV9 |
| Central nervous system | AAV5, AAV1, AAV4 |
| RPE | AAV5, AAV4 |
| Photoreceptor cells | AAV5 |
| Lung | AAV9 |
| Heart | AAV8 |
| Pancreas | AAV8 |
| Kidney | AAV2 |

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the disclosure, the guide RNA and/or site-directed modifying polypeptide and/or donor polynucleotide are employed to modify cellular DNA in vivo, again for purposes such as gene therapy, e.g., to treat a disease or as an antiviral, antipathogenic, or anticancer therapeutic, for the production of genetically modified organisms in agriculture, or for biological research. In these in vivo embodiments, a guide RNA and/or site-directed modifying polypeptide and/or donor polynucleotide are administered directly to the individual. A guide RNA and/or site-directed modifying polypeptide and/or donor polynucleotide may be administered by any of a number of well-known methods in the art for the administration of peptides, small molecules and nucleic acids to a subject. A guide RNA and/or site-directed modifying polypeptide and/or donor polynucleotide can be incorporated into a variety of formulations. More particularly, a guide RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a guide RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the US Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a guide RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the BBB entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g., through an Ommaya reservoir (see e.g., U.S. Pat. Nos. 5,222,982 and 5,385,582, incorporated herein by reference); by bolus injection, e.g., by a syringe, e.g., intravitreally or intracranially; by continuous infusion, e.g., by cannulation, e.g., with convection (see e.g., US Application No. 20070254842, incorporated here by reference); or by implanting a device upon which the agent has been reversibly affixed (see e.g., US Application Nos. 20080081064 and 20090196903, incorporated herein by reference).

Typically, an effective amount of a guide RNA and/or site-directed modifying polypeptide and/or donor polynucleotide are provided. As discussed above with regard to ex vivo methods, an effective amount or effective dose of a guide RNA and/or site-directed modifying polypeptide and/or donor polynucleotide in vivo is the amount to induce a 2 fold increase or more in the amount of recombination observed between two homologous sequences relative to a negative control, e.g., a cell contacted with an empty vector or irrelevant polypeptide. The amount of recombination may be measured by any convenient method, e.g., as described above and known in the art. The calculation of the effective amount or effective dose of a guide RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally-administered dose, given the greater body of fluid into which the therapeutic composition is being administered. Similarly, compositions, which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a guide RNA and/or site-directed modifying polypeptide and/or donor polynucleotide may be obtained from a suitable commercial source. As a general proposition, the total pharmaceutically effective amount of a guide RNA and/or site-directed modifying polypeptide and/or donor polynucleotide administered parenterally per dose will be in a range that can be measured by a dose response curve.

Therapies based on a guide RNA and/or site-directed modifying polypeptide and/or donor polynucleotides, i.e., preparations of a guide RNA and/or site-directed modifying polypeptide and/or donor polynucleotide to be used for therapeutic administration, must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The therapies based on a guide RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized. The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions that are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Genetically Modified Host Cells

The present disclosure provides genetically modified host cells, including isolated genetically modified host cells, where a genetically modified host cell comprises (has been genetically modified with: 1) an exogenous guide RNA; 2) an exogenous nucleic acid comprising a nucleotide sequence encoding a guide RNA; 3) an exogenous site-directed modifying polypeptide (e.g., a naturally occurring Cpf1; a modified, i.e., mutated or variant, Cpf1; a chimeric Cpf1; etc.); 4) an exogenous nucleic acid comprising a nucleotide sequence encoding a site-directed modifying polypeptide; or 5) any combination of the above. A genetically modified cell is generated by genetically modifying a host cell with, for example: 1) an exogenous guide RNA; 2) an exogenous nucleic acid comprising a nucleotide sequence encoding a guide RNA; 3) an exogenous site-directed modifying polypeptide; 4) an exogenous nucleic acid comprising a nucleotide sequence encoding a site-directed modifying polypeptide; or 5) any combination of the above.).

All cells suitable to be a target cell are also suitable to be a genetically modified host cell. For example, a genetically modified host cells of interest can be a cell from any organism (e.g., a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc.

In some embodiments, a genetically modified host cell has been genetically modified with an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.). The DNA of a genetically modified host cell can be targeted for modification by introducing into the cell a guide RNA (or a DNA encoding a guide RNA, which determines the genomic location/sequence to be modified) and optionally a donor nucleic acid. In some embodiments, the nucleotide sequence encoding a site-directed modifying polypeptide is operably linked to an inducible promoter (e.g., heat shock promoter, Tetracycline-regulated promoter, Steroid-regulated promoter, Metal-regulated promoter, estrogen receptor-regulated promoter, etc.). In some embodiments, the nucleotide sequence encoding a site-directed modifying polypeptide is operably linked to a spatially restricted and/or temporally restricted promoter (e.g., a tissue specific promoter, a cell type specific promoter, etc.). In some embodiments, the nucleotide sequence encoding a site-directed modifying polypeptide is operably linked to a constitutive promoter.

In some embodiments, a genetically modified host cell is in vitro. In some embodiments, a genetically modified host cell is in vivo. In some embodiments, a genetically modified host cell is a prokaryotic cell or is derived from a prokaryotic cell. In some embodiments, a genetically modified host cell is a bacterial cell or is derived from a bacterial cell. In some embodiments, a genetically modified host cell is an archaeal cell or is derived from an archaeal cell. In some embodiments, a genetically modified host cell is a eukaryotic cell or is derived from a eukaryotic cell. In some embodiments, a genetically modified host cell is a plant cell or is derived from a plant cell. In some embodiments, a genetically modified host cell is an animal cell or is derived from an animal cell. In some embodiments, a genetically modified host cell is an invertebrate cell or is derived from an invertebrate cell. In some embodiments, a genetically modified host cell is a vertebrate cell or is derived from a vertebrate cell. In some embodiments, a genetically modified host cell is a mammalian cell or is derived from a mammalian cell. In some embodiments, a genetically modified host cell is a rodent cell or is derived from a rodent cell. In some embodiments, a genetically modified host cell is a human cell or is derived from a human cell.

The present disclosure further provides progeny of a genetically modified cell, where the progeny can comprise the same exogenous nucleic acid or polypeptide as the genetically modified cell from which it was derived. The present disclosure further provides a composition comprising a genetically modified host cell.

Genetically Modified Stem Cells and Genetically Modified Progenitor Cells

In some embodiments, a genetically modified host cell is a genetically modified stem cell or progenitor cell. Suitable host cells include, e.g., stem cells (adult stem cells, embryonic stem cells, iPS cells, etc.) and progenitor cells (e.g., cardiac progenitor cells, neural progenitor cells, etc.). Suitable host cells include mammalian stem cells and progenitor cells, including, e.g., rodent stem cells, rodent progenitor cells, human stem cells, human progenitor cells, etc. Suitable host cells include in vitro host cells, e.g., isolated host cells.

In some embodiments, a genetically modified host cell comprises an exogenous guide RNA nucleic acid. In some embodiments, a genetically modified host cell comprises an exogenous nucleic acid comprising a nucleotide sequence encoding a guide RNA. In some embodiments, a genetically modified host cell comprises an exogenous site-directed modifying polypeptide (e.g., a naturally occurring Cpf1; a modified, i.e., mutated or variant, Cpf1; a chimeric Cpf1; etc.). In some embodiments, a genetically modified host cell comprises an exogenous nucleic acid comprising a nucleotide sequence encoding a site-directed modifying polypeptide. In some embodiments, a genetically modified host cell comprises exogenous nucleic acid comprising a nucleotide sequence encoding 1) a guide RNA and 2) a site-directed modifying polypeptide.

In some cases, the site-directed modifying polypeptide comprises an amino acid sequence having at least about 75%, at least about 80%, at least about 85%, at least about 90%, at least about 95%, at least about 99%, or 100% amino acid sequence identity to any of the sequences in FIG. 1, or an active portion thereof which is at least 100, 150, 200, 300, 350, 400, or 500 amino acids long. In some embodiments, the active portion is the RNase domain. In other embodiments, the active portion is the DNase domain.

Compositions

The present disclosure provides a composition comprising a guide RNA and/or a site-directed modifying polypeptide. In some cases, the site-directed modifying polypeptide is a chimeric polypeptide. A composition is useful for carrying out a method of the present disclosure, e.g., a method for site-specific modification of a target DNA; a method for site-specific modification of a polypeptide associated with a target DNA; etc.

Compositions Comprising a Guide RNA

The present disclosure provides a composition comprising a guide RNA. The composition can comprise, in addition to the guide RNA, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), MES sodium salt, 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; and the like. For example, in some cases, a composition comprises a guide RNA and a buffer for stabilizing nucleic acids.

In some embodiments, a guide RNA present in a composition is pure, e.g., at least about 75%, at least about 80%, at least about 85%, at least about 90%, at least about 95%, at least about 98%, at least about 99%, or more than 99% pure, where “% purity” means that guide RNA is the recited percent free from other macromolecules, or contaminants that may be present during the production of the guide RNA.

Compositions Comprising a Chimeric Polypeptide

The present disclosure provides a composition a chimeric polypeptide. The composition can comprise, in addition to the guide RNA, one or more of: a salt, e.g., NaCl, MgCl₂, KCl, MgSO₄, etc.; a buffering agent, e.g., a Tris buffer, HEPES, MES, MES sodium salt, MOPS, TAPS, etc.; a solubilizing agent; a detergent, e.g., a non-ionic detergent such as Tween-20, etc.; a protease inhibitor; a reducing agent (e.g., dithiothreitol); and the like.

In some embodiments, a chimeric polypeptide present in a composition is pure, e.g., at least about 75%, at least about 80%, at least about 85%, at least about 90%, at least about 95%, at least about 98%, at least about 99%, or more than 99% pure, where “% purity” means that the site-directed modifying polypeptide is the recited percent free from other proteins, other macromolecules, or contaminants that may be present during the production of the chimeric polypeptide.

Compositions Comprising a Guide RNA and a Site-Directed Modifying Polypeptide

The present disclosure provides a composition comprising: (i) a guide RNA or a DNA polynucleotide encoding the same; and ii) a site-directed modifying polypeptide, or a polynucleotide encoding the same. In some cases, the site-directed modifying polypeptide is a chimeric site-directed modifying polypeptide. In other cases, the site-directed modifying polypeptide is a naturally occurring site-directed modifying polypeptide. In some instances, the site-directed modifying polypeptide exhibits enzymatic activity that modifies a target DNA. In other cases, the site-directed modifying polypeptide exhibits enzymatic activity that modifies a polypeptide that is associated with a target DNA. In still other cases, the site-directed modifying polypeptide modulates transcription of the target DNA.

The present disclosure provides a composition comprising: (i) a guide RNA, as described above, or a DNA polynucleotide encoding the same, the guide RNA comprising: (a) a first segment comprising a nucleotide sequence that is complementary to a sequence in a target DNA; and (b) a second segment that interacts with a site-directed modifying polypeptide; and (ii) the site-directed modifying polypeptide, or a polynucleotide encoding the same, the site-directed modifying polypeptide comprising: (a) an RNA-binding portion that interacts with the guide RNA; and (b) an activity portion that exhibits site-directed enzymatic activity, wherein the site of enzymatic activity is determined by the guide RNA.

In some instances, a composition comprises: (i) a guide RNA, the guide RNA comprising: (a) a first segment comprising a nucleotide sequence that is complementary to a sequence in a target DNA; and (b) a second segment that interacts with a site-directed modifying polypeptide; and (ii) the site-directed modifying polypeptide, the site-directed modifying polypeptide comprising: (a) an RNA-binding portion that interacts with the guide RNA; and (b) an activity portion that exhibits site-directed enzymatic activity, wherein the site of enzymatic activity is determined by the guide RNA.

In other embodiments, a composition comprises: (i) a polynucleotide encoding a guide RNA, the guide RNA comprising: (a) a first segment comprising a nucleotide sequence that is complementary to a sequence in a target DNA; and (b) a second segment that interacts with a site-directed modifying polypeptide; and (ii) a polynucleotide encoding the site-directed modifying polypeptide, the site-directed modifying polypeptide comprising: (a) an RNA-binding portion that interacts with the guide RNA; and (b) an activity portion that exhibits site-directed enzymatic activity, wherein the site of enzymatic activity is determined by the guide RNA.

The present disclosure provides a composition comprising: (i) a guide RNA, or a DNA polynucleotide encoding the same, the guide RNA comprising: (a) a first segment comprising a nucleotide sequence that is complementary to a sequence in a target DNA; and (b) a second segment that interacts with a site-directed modifying polypeptide; and (ii) the site-directed modifying polypeptide, or a polynucleotide encoding the same, the site-directed modifying polypeptide comprising: (a) an RNA-binding portion that interacts with the guide RNA; and (b) an activity portion that modulates transcription within the target DNA, wherein the site of modulated transcription within the target DNA is determined by the guide RNA.

For example, in some cases, a composition comprises: (i) a guide RNA, the guide RNA comprising: (a) a first segment comprising a nucleotide sequence that is complementary to a sequence in a target DNA; and (b) a second segment that interacts with a site-directed modifying polypeptide; and (ii) the site-directed modifying polypeptide, the site-directed modifying polypeptide comprising: (a) an RNA-binding portion that interacts with the guide RNA; and (b) an activity portion that modulates transcription within the target DNA, wherein the site of modulated transcription within the target DNA is determined by the guide RNA.

As another example, in some cases, a composition comprises: (i) a DNA polynucleotide encoding a guide RNA, the guide RNA comprising: (a) a first segment comprising a nucleotide sequence that is complementary to a sequence in a target DNA; and (b) a second segment that interacts with a site-directed modifying polypeptide; and (ii) a polynucleotide encoding the site-directed modifying polypeptide, the site-directed modifying polypeptide comprising: (a) an RNA-binding portion that interacts with the guide RNA; and (b) an activity portion that modulates transcription within the target DNA, wherein the site of modulated transcription within the target DNA is determined by the guide RNA. A composition can comprise, in addition to i) a guide RNA, or a DNA polynucleotide encoding the same; and ii) a site-directed modifying polypeptide, or a polynucleotide encoding the same, one or more of: a salt, e.g., NaCl, MgCl₂, KCl, MgSO₄, etc.; a buffering agent, e.g., a Tris buffer, HEPES, MES, MES sodium salt, MOPS, TAPS, etc.; a solubilizing agent; a detergent, e.g., a non-ionic detergent such as Tween-20, etc.; a protease inhibitor; a reducing agent (e.g., dithiothreitol); and the like.

In some cases, the components of the composition are individually pure, e.g., each of the components is at least about 75%, at least about 80%, at least about 90%, at least about 95%, at least about 98%, at least about 99%, or at least 99%, pure. In some cases, the individual components of a composition are pure before being added to the composition.

For example, in some embodiments, a site-directed modifying polypeptide present in a composition is pure, e.g., at least about 75%, at least about 80%, at least about 85%, at least about 90%, at least about 95%, at least about 98%, at least about 99%, or more than 99% pure, where “% purity” means that the site-directed modifying polypeptide is the recited percent free from other proteins (e.g., proteins other than the site-directed modifying polypeptide), other macromolecules, or contaminants that may be present during the production of the site-directed modifying polypeptide.

Kits

The present disclosure provides kits for carrying out a method. A kit can include one or more of: a site-directed modifying polypeptide; a nucleic acid comprising a nucleotide encoding a site-directed modifying polypeptide; a guide RNA; a nucleic acid comprising a nucleotide sequence encoding a guide RNA. A kit may comprise a complex that comprises two or more of: a site-directed modifying polypeptide; a nucleic acid comprising a nucleotide encoding a site-directed modifying polypeptide; a guide RNA; a nucleic acid comprising a nucleotide sequence encoding a guide RNA. In some embodiments, a kit comprises a site-directed modifying polypeptide, or a polynucleotide encoding the same. In some embodiments, the site-directed modifying polypeptide comprises: (a) an RNA-binding portion that interacts with the guide RNA; and (b) an activity portion that modulates transcription within the target DNA, wherein the guide RNA determines the site of modulated transcription within the target DNA. In some cases, the activity portion of the site-directed modifying polypeptide exhibits reduced or inactivated nuclease activity. In some cases, the site-directed modifying polypeptide is a chimeric site-directed modifying polypeptide.

In some embodiments, a kit comprises: a site-directed modifying polypeptide, or a polynucleotide encoding the same, and a reagent for reconstituting and/or diluting the site-directed modifying polypeptide. In other embodiments, a kit comprises a nucleic acid (e.g., DNA, RNA) comprising a nucleotide encoding a site-directed modifying polypeptide. In some embodiments, a kit comprises: a nucleic acid (e.g., DNA, RNA) comprising a nucleotide encoding a site-directed modifying polypeptide; and a reagent for reconstituting and/or diluting the site-directed modifying polypeptide.

A kit comprising a site-directed modifying polypeptide, or a polynucleotide encoding the same, can further include one or more additional reagents, where such additional reagents can be selected from: a buffer for introducing the site-directed modifying polypeptide into a cell; a wash buffer; a control reagent; a control expression vector or RNA polynucleotide; a reagent for in vitro production of the site-directed modifying polypeptide from DNA, and the like. In some cases, the site-directed modifying polypeptide included in a kit is a chimeric site-directed modifying polypeptide, as described above.

In some embodiments, a kit comprises a guide RNA, or a DNA polynucleotide encoding the same, the guide RNA comprising: (a) a first segment comprising a nucleotide sequence that is complementary to a sequence in a target DNA; and (b) a second segment that interacts with a site-directed modifying polypeptide. In some embodiments, a kit comprises: (i) a guide RNA, or a DNA polynucleotide encoding the same, the guide RNA comprising: (a) a first segment comprising a nucleotide sequence that is complementary to a sequence in a target DNA; and (b) a second segment that interacts with a site-directed modifying polypeptide; and (ii) a site-directed modifying polypeptide, or a polynucleotide encoding the same, the site-directed modifying polypeptide comprising: (a) an RNA-binding portion that interacts with the guide RNA; and (b) an activity portion that exhibits site-directed enzymatic activity, wherein the site of enzymatic activity is determined by the guide RNA. In some embodiments, the activity portion of the site-directed modifying polypeptide does not exhibit enzymatic activity (comprises an inactivated nuclease, e.g., via mutation). In some cases, the kit comprises a guide RNA and a site-directed modifying polypeptide. In other cases, the kit comprises: (i) a nucleic acid comprising a nucleotide sequence encoding a guide RNA; and (ii) a nucleic acid comprising a nucleotide sequence encoding site-directed modifying polypeptide. As another example, a kit can include: (i) a guide RNA, or a DNA polynucleotide encoding the same, comprising: (a) a first segment comprising a nucleotide sequence that is complementary to a sequence in a target DNA; and (b) a second segment that interacts with a site-directed modifying polypeptide; and (ii) the site-directed modifying polypeptide, or a polynucleotide encoding the same, comprising: (a) an RNA-binding portion that interacts with the guide RNA; and (b) an activity portion that that modulates transcription within the target DNA, wherein the site of modulated transcription within the target DNA is determined by the guide RNA In some cases, the kit comprises: (i) a guide RNA; and a site-directed modifying polypeptide. In other cases, the kit comprises: (i) a nucleic acid comprising a nucleotide sequence encoding a guide RNA; and (ii) a nucleic acid comprising a nucleotide sequence encoding site-directed modifying polypeptide. The present disclosure provides a kit comprising: (1) a recombinant expression vector comprising (i) a nucleotide sequence encoding a guide RNA, wherein the guide RNA comprises: (a) a first segment comprising a nucleotide sequence that is complementary to a sequence in a target DNA; and (b) a second segment that interacts with a site-directed modifying polypeptide; and (ii) a nucleotide sequence encoding the site-directed modifying polypeptide, wherein the site-directed modifying polypeptide comprises: (a) an RNA-binding portion that interacts with the guide RNA; and (b) an activity portion that exhibits site-directed enzymatic activity, wherein the site of enzymatic activity is determined by the guide RNA; and (2) a reagent for reconstitution and/or dilution of the expression vector.

The present disclosure provides a kit comprising: (1) a recombinant expression vector comprising: (i) a nucleotide sequence encoding a guide RNA, wherein the guide RNA comprises: (a) a first segment comprising a nucleotide sequence that is complementary to a sequence in a target DNA; and (b) a second segment that interacts with a site-directed modifying polypeptide; and (ii) a nucleotide sequence encoding the site-directed modifying polypeptide, wherein the site-directed modifying polypeptide comprises: (a) an RNA-binding portion that interacts with the guide RNA; and (b) an activity portion that modulates transcription within the target DNA, wherein the site of modulated transcription within the target DNA is determined by the guide RNA; and (2) a reagent for reconstitution and/or dilution of the recombinant expression vector.

The present disclosure provides a kit comprising: (1) a recombinant expression vector comprising a nucleic acid comprising a nucleotide sequence that encodes a DNA targeting RNA comprising: (i) a first segment comprising a nucleotide sequence that is complementary to a sequence in a target DNA; and (ii) a second segment that interacts with a site-directed modifying polypeptide; and (2) a reagent for reconstitution and/or dilution of the recombinant expression vector. In some embodiments of this kit, the kit comprises: a recombinant expression vector comprising a nucleotide sequence that encodes a site-directed modifying polypeptide, wherein the site-directed modifying polypeptide comprises: (a) an RNA-binding portion that interacts with the guide RNA; and (b) an activity portion that exhibits site-directed enzymatic activity, wherein the site of enzymatic activity is determined by the guide RNA. In other embodiments of this kit, the kit comprises: a recombinant expression vector comprising a nucleotide sequence that encodes a site-directed modifying polypeptide, wherein the site-directed modifying polypeptide comprises: (a) an RNA-binding portion that interacts with the guide RNA; and (b) an activity portion that modulates transcription within the target DNA, wherein the site of modulated transcription within the target DNA is determined by the guide RNA.

In some embodiments of any of the above kits, the kit comprises a single-molecule guide RNA. In some embodiments of any of the above kits, the kit comprises two or more single-molecule guide RNAs. In some embodiments of any of the above kits, a guide RNA (e.g., including two or more guide RNAs) can be provided as an array (e.g., an array of RNA molecules, an array of DNA molecules encoding the guide RNA(s), etc.). Such kits can be useful, for example, for use in conjunction with the above described genetically modified host cells that comprise a site-directed modifying polypeptide. In some embodiments of any of the above kits, the kit further comprises a donor polynucleotide to effect the desired genetic modification. Components of a kit can be in separate containers; or can be combined in a single container.

In some cases, a kit further comprises one or more variant Cpf1 site-directed polypeptides that exhibit reduced endodeoxyribonuclease activity relative to wild-type Cpf1.

In some cases, a kit further comprises one or more nucleic acids comprising a nucleotide sequence encoding a variant Cpf1 site-directed polypeptide that exhibits reduced endodeoxyribonuclease activity relative to wild-type Cpf1.

Any of the above-described kits can further include one or more additional reagents, where such additional reagents can be selected from: a dilution buffer; a reconstitution solution; a wash buffer; a control reagent; a control expression vector or RNA polynucleotide; a reagent for in vitro production of the site-directed modifying polypeptide from DNA, and the like.

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

Non-Human Genetically Modified Organisms

In some embodiments, a genetically modified host cell has been genetically modified with an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.). If such a cell is a eukaryotic single-cell organism, then the modified cell can be considered a genetically modified organism. In some embodiments, the non-human genetically modified organism is a Cpf1 transgenic multicellular organism.

In some embodiments, a genetically modified non-human host cell (e.g., a cell that has been genetically modified with an exogenous nucleic acid comprising a nucleotide sequence encoding a site-directed modifying polypeptide, e.g., a naturally occurring Cpf1; a modified, i.e., mutated or variant, Cpf1; a chimeric Cpf1; etc.) can generate a genetically modified nonhuman organism (e.g., a mouse, a fish, a frog, a fly, a worm, etc.). For example, if the genetically modified host cell is a pluripotent stem cell (i.e., PSC) or a germ cell (e.g., sperm, oocyte, etc.), an entire genetically modified organism can be derived from the genetically modified host cell. In some embodiments, the genetically modified host cell is a pluripotent stem cell (e.g., ESC, iPSC, pluripotent plant stem cell, etc.) or a germ cell (e.g., sperm cell, oocyte, etc.), either in vivo or in vitro that can give rise to a genetically modified organism. In some embodiments the genetically modified host cell is a vertebrate PSC (e.g., ESC, iPSC, etc.) and is used to generate a genetically modified organism (e.g., by injecting a PSC into a blastocyst to produce a chimeric/mosaic animal, which could then be mated to generate non-chimeric/non-mosaic genetically modified organisms; grafting in the case of plants; etc.). Any convenient method/protocol for producing a genetically modified organism, including the methods described herein, is suitable for producing a genetically modified host cell comprising an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.). Methods of producing genetically modified organisms are known in the art. For example, see Cho et al., Curr Protoc Cell Biol. 2009 March; Chapter 19:Unit 19.11: Generation of transgenic mice; Gama et al., Brain Struct Funct. 2010 March; 214(2-3):91-109. Epub 2009 Nov. 25: Animal transgenesis: an overview; Husaini et al., GM Crops. 2011 June-December; 2(3):150-62. Epub 2011 June 1: Approaches for gene targeting and targeted gene expression in plants.

In some embodiments, a genetically modified organism comprises a target cell for methods of the invention, and thus can be considered a source for target cells. For example, if a genetically modified cell comprising an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.) is used to generate a genetically modified organism, then the cells of the genetically modified organism comprise the exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.). In some such embodiments, the DNA of a cell or cells of the genetically modified organism can be targeted for modification by introducing into the cell or cells a guide RNA (or a DNA encoding a guide RNA) and optionally a donor nucleic acid. For example, the introduction of a guide RNA (or a DNA encoding a guide RNA) into a subset of cells (e.g., brain cells, intestinal cells, kidney cells, lung cells, blood cells, etc.) of the genetically modified organism can target the DNA of such cells for modification, the genomic location of which will depend on the DNA-targeting sequence of the introduced guide RNA.

In some embodiments, a genetically modified organism is a source of target cells for methods of the invention. For example, a genetically modified organism comprising cells that are genetically modified with an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.) can provide a source of genetically modified cells, for example PSCs (e.g., ESCs, iPSCs, sperm, oocytes, etc.), neurons, progenitor cells, cardiomyocytes, etc.

In some embodiments, a genetically modified cell is a PSC comprising an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.). As such, the PSC can be a target cell such that the DNA of the PSC can be targeted for modification by introducing into the PSC a guide RNA (or a DNA encoding a guide RNA) and optionally a donor nucleic acid, and the genomic location of the modification will depend on the DNA-targeting sequence of the introduced guide RNA. Thus, in some embodiments, the methods described herein can be used to modify the DNA (e.g., delete and/or replace any desired genomic location) of PSCs derived from a genetically modified organism. Such modified PSCs can then be used to generate organisms having both (i) an exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.) and (ii) a DNA modification that was introduced into the PSC.

An exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.) can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters (e.g., CMV promoter), inducible promoters (e.g., heat shock promoter, Tetracycline-regulated promoter, Steroid-regulated promoter, Metal-regulated promoter, estrogen receptor-regulated promoter, etc.), spatially restricted and/or temporally restricted promoters (e.g., a tissue specific promoter, a cell type specific promoter, etc.), etc.

A genetically modified organism (e.g., an organism whose cells comprise a nucleotide sequence encoding a site-directed modifying polypeptide, e.g., a naturally occurring Cpf1; a modified, i.e., mutated or variant, Cpf1; a chimeric Cpf1; etc.) can be any organism including for example, a plant; algae; an invertebrate (e.g., a cnidarian, an echinoderm, a worm, a fly, etc.); a vertebrate (e.g., a fish (e.g., zebrafish, puffer fish, gold fish, etc.), an amphibian (e.g., salamander, frog, etc.), a reptile, a bird, a mammal, etc.); an ungulate (e.g., a goat, a pig, a sheep, a cow, etc.); a rodent (e.g., a mouse, a rat, a hamster, a guinea pig); a lagomorpha (e.g., a rabbit); etc.

In some cases, the site-directed modifying polypeptide comprises an amino acid sequence having at least about 75%, at least about 80%, at least about 85%, at least about 90%, at least about 95%, at least about 99%, or 100% amino acid sequence identity to any one of SEQ ID NOs:2-. 10

Transgenic Non-Human Animals

As described above, in some embodiments, a nucleic acid (e.g., a nucleotide sequence encoding a site-directed modifying polypeptide, e.g., a naturally occurring Cpf1; a modified, i.e., mutated or variant, Cpf1; a chimeric Cpf1; etc.) or a recombinant expression vector is used as a transgene to generate a transgenic animal that produces a site-directed modifying polypeptide. Thus, the present disclosure further provides a transgenic non-human animal, which animal comprises a transgene comprising a nucleic acid comprising a nucleotide sequence encoding a site-directed modifying polypeptide, e.g., a naturally occurring Cpf1; a modified, i.e., mutated or variant, Cpf1; a chimeric Cpf1; etc., as described above. In some embodiments, the genome of the transgenic non-human animal comprises a nucleotide sequence encoding a site-directed modifying polypeptide. In some embodiments, the transgenic non-human animal is homozygous for the genetic modification. In some embodiments, the transgenic non-human animal is heterozygous for the genetic modification. In some embodiments, the transgenic non-human animal is a vertebrate, for example, a fish (e.g., zebra fish, gold fish, puffer fish, cave fish, etc.), an amphibian (frog, salamander, etc.), a bird (e.g., chicken, turkey, etc.), a reptile (e.g., snake, lizard, etc.), a mammal (e.g., an ungulate, e.g., a pig, a cow, a goat, a sheep, etc.; a lagomorph (e.g., a rabbit); a rodent (e.g., a rat, a mouse); a nonhuman primate; etc.), etc.

An exogenous nucleic acid comprising a nucleotide sequence encoding a site-directed modifying polypeptide (e.g., a naturally occurring Cpf1; a modified, i.e., mutated or variant, Cpf1; a chimeric Cpf1; etc.) can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters (e.g., CMV promoter), inducible promoters (e.g., heat shock promoter, Tetracycline-regulated promoter, Steroid-regulated promoter, Metal-regulated promoter, estrogen receptor-regulated promoter, etc.), spatially restricted and/or temporally restricted promoters (e.g., a tissue specific promoter, a cell type specific promoter, etc.), etc.

Transgenic Plants

As described above, in some embodiments, a nucleic acid (e.g., a nucleotide sequence encoding a site-directed modifying polypeptide, e.g., a naturally occurring Cpf1; a modified, i.e., mutated or variant, Cpf1; a chimeric Cpf1; etc.) or a recombinant expression vector is used as a transgene to generate a transgenic plant that produces a site-directed modifying polypeptide. Thus, the present disclosure further provides a transgenic plant, which plant comprises a transgene comprising a nucleic acid comprising a nucleotide sequence encoding site-directed modifying polypeptide, e.g., a naturally occurring Cpf1; a modified, i.e., mutated or variant, Cpf1; a chimeric Cpf1; etc., as described above. In some embodiments, the genome of the transgenic plant comprises a nucleic acid. In some embodiments, the transgenic plant is homozygous for the genetic modification. In some embodiments, the transgenic plant is heterozygous for the genetic modification.

Methods of introducing exogenous nucleic acids into plant cells are well known in the art. Such plant cells are considered “transformed,” as defined above. Suitable methods include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e., in vitro, ex vivo, or in vivo). Transformation methods based upon the soil bacterium Agrobacterium tumefaciens are particularly useful for introducing an exogenous nucleic acid molecule into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993).

Microprojectile-mediated transformation also can be used to produce a transgenic plant. This method, first described by Klein et al. (Nature 327:70-73 (1987)), relies on microprojectiles such as gold or tungsten that are coated with the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid may be introduced into a plant in a manner such that the nucleic acid is able to enter a plant cell(s), e.g., via an in vivo or ex vivo protocol. By “in vivo,” it is meant in the nucleic acid is administered to a living body of a plant e.g., infiltration. By “ex vivo” it is meant that cells or explants are modified outside of the plant, and then such cells or organs are regenerated to a plant. A number of vectors suitable for stable transformation of plant cells or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucl Acid Res. 12: 8711-8721, Klee (1985) Bio/Technolo 3: 637-642. Alternatively, non-Ti vectors can be used to transfer the DNA into plants and cells by using free DNA delivery techniques. By using these methods transgenic plants such as wheat, rice (Christou (1991) Bio/Technology 9:957-9 and 4462) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol 102: 1077-1084; Vasil (1993) Bio/Technolo 10: 667-674; Wan and Lemeaux (1994) Plant Physiol 104: 37-48 and for Agrobacterium-mediated DNA transfer (Ishida et al. (1996) Nature Biotech 14: 745-750). Exemplary methods for introduction of DNA into chloroplasts are biolistic bombardment, polyethylene glycol transformation of protoplasts, and microinjection (Daniell et al. Nat. Biotechnol 16:345-348, 1998; Staub et al. Nat. Biotechnol 18: 333-338, 2000; O'Neill et al. Plant J. 3:729-738, 1993; Knoblauch et al. Nat. Biotechnol 17: 906-909; U.S. Pat. Nos. 5,451,513, 5,545,817, 5,545,818, and 5,576,198; in Intl. Application No. WO 95/16783; and in Boynton et al., Methods in Enzymology 217: 510-536 (1993), Svab et al., Proc. Natl. Acad. Sci. USA 90: 913-917 (1993), and McBride et al., Proc. Nati. Acad. Sci. USA 91: 7301-7305 (1994)). Any vector suitable for the methods of biolistic bombardment, polyethylene glycol transformation of protoplasts and microinjection will be suitable as a targeting vector for chloroplast transformation. Any double stranded DNA vector may be used as a transformation vector, especially when the method of introduction does not utilize Agrobacterium.

Plants, which can be genetically modified, include grains, forage crops, fruits, vegetables, oil seed crops, palms, forestry, and vines. Specific examples of plants which can be modified follow: maize, banana, peanut, field peas, sunflower, tomato, canola, tobacco, wheat, barley, oats, potato, soybeans, cotton, carnations, sorghum, lupin and rice.

Also provided by the disclosure are transformed plant cells, tissues, plants and products that contain the transformed plant cells. A feature of the transformed cells, and tissues and products that include the same is the presence of a nucleic acid integrated into the genome, and production by plant cells of a site-directed modifying polypeptide, e.g., a naturally occurring Cpf1; a modified, i.e., mutated or variant, Cpf1; a chimeric Cpf1; etc. Recombinant plant cells of the present invention are useful as populations of recombinant cells, or as a tissue, seed, whole plant, stem, fruit, leaf, root, flower, stem, tuber, grain, animal feed, a field of plants, and the like.

A nucleic acid comprising a nucleotide sequence encoding a site-directed modifying polypeptide (e.g., a naturally occurring Cpf1; a modified, i.e., mutated or variant, Cpf1; a chimeric Cpf1; etc.) can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters, inducible promoters, spatially restricted and/or temporally restricted promoters, etc.

The present disclosure provides methods of modulating transcription of a target nucleic acid in a host cell. The methods generally involve contacting the target nucleic acid with an enzymatically inactive Cpf1 polypeptide and a guide RNA. The methods are useful in a variety of applications, which are also provided.

A transcriptional modulation method of the present disclosure overcomes some of the drawbacks of methods involving RNAi. A transcriptional modulation method of the present disclosure finds use in a wide variety of applications, including research applications, drug discovery (e.g., high throughput screening), target validation, industrial applications (e.g., crop engineering; microbial engineering, etc.), diagnostic applications, therapeutic applications, and imaging techniques.

Methods of Modulating Transcription

The present disclosure provides a method of selectively modulating transcription of a target DNA in a host cell. The method generally involves: a) introducing into the host cell: i) a guide RNA, or a nucleic acid comprising a nucleotide sequence encoding the guide RNA; and ii) a variant Cpf1 site-directed polypeptide (“variant Cpf1 polypeptide”), or a nucleic acid comprising a nucleotide sequence encoding the variant Cpf1 polypeptide, where the variant Cpf1 polypeptide exhibits reduced endodeoxyribonuclease activity.

The guide RNA (also referred to herein as “guide RNA”; or “gRNA”) comprises: i) a first segment comprising a nucleotide sequence that is complementary to a target sequence in a target DNA; ii) a second segment that interacts with a site-directed polypeptide; and iii) a transcriptional terminator. The first segment, comprising a nucleotide sequence that is complementary to a target sequence in a target DNA, is referred to herein as a “targeting segment”. The second segment, which interacts with a site-directed polypeptide, is also referred to herein as a “protein-binding sequence” or “dCpf1-binding hairpin,” or “dCpf1 handle.” By “segment” it is meant a segment/section/region of a molecule, e.g., a contiguous stretch of nucleotides in an RNA. The definition of “segment,” unless otherwise specifically defined in a particular context, is not limited to a specific number of total base pairs, and may include regions of RNA molecules that are of any total length and may or may not include regions with complementarity to other molecules. The variant Cpf1 site-directed polypeptide comprises: i) an RNA-binding portion that interacts with the guide RNA; and an activity portion that exhibits reduced endodeoxyribonuclease activity.

The guide RNA and the variant Cpf1 polypeptide form a complex in the host cell; the complex selectively modulates transcription of a target DNA in the host cell.

In some cases, a transcription modulation method of the present disclosure provides for selective modulation (e.g., reduction or increase) of a target nucleic acid in a host cell. For example, “selective” reduction of transcription of a target nucleic acid reduces transcription of the target nucleic acid by at least about 10%, at least about 20%, at least about 30%, at least about 40%, at least about 50%, at least about 60%, at least about 70%, at least about 80%, at least about 90%, or greater than 90%, compared to the level of transcription of the target nucleic acid in the absence of a guide RNA/variant Cpf1 polypeptide complex. Selective reduction of transcription of a target nucleic acid reduces transcription of the target nucleic acid, but does not substantially reduce transcription of a non-target nucleic acid, e.g., transcription of a non-target nucleic acid is reduced, if at all, by less than 10% compared to the level of transcription of the non-target nucleic acid in the absence of the guide RNA/variant Cpf1 polypeptide complex.

Increased Transcription

“Selective” increased transcription of a target DNA can increase transcription of the target DNA by at least about 1.1 fold (e.g., at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, or at least about 20-fold) compared to the level of transcription of the target DNA in the absence of a guide RNA/variant Cpf1 polypeptide complex. Selective increase of transcription of a target DNA increases transcription of the target DNA, but does not substantially increase transcription of a non-target DNA, e.g., transcription of a non-target DNA is increased, if at all, by less than about 5-fold (e.g., less than about 4-fold, less than about 3-fold, less than about 2-fold, less than about 1.8-fold, less than about 1.6-fold, less than about 1.4-fold, less than about 1.2-fold, or less than about 1.1-fold) compared to the level of transcription of the non-targeted DNA in the absence of the guide RNA/variant Cpf1 polypeptide complex.

As a non-limiting example, increased transcription can be achieved by fusing dCpf1 to a heterologous sequence. Suitable fusion partners include, but are not limited to, a polypeptide that provides an activity that indirectly increases transcription by acting directly on the target DNA or on a polypeptide (e.g., a histone or other DNA-binding protein) associated with the target DNA. Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

Additional suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.).

A non-limiting example of a method using a dCpf1 fusion protein to increase transcription in a prokaryote includes a modification of the bacterial one-hybrid (B1H) or two-hybrid (B2H) system. In the B1H system, a DNA binding domain (BD) is fused to a bacterial transcription activation domain (AD, e.g., the alpha subunit of the Escherichia coli RNA polymerase (RNAPa)). Thus, a dCpf1 can be fused to a heterologous sequence comprising an AD. When the dCpf1 fusion protein arrives at the upstream region of a promoter (targeted there by the guide RNA) the AD (e.g., RNAPa) of the dCpf1 fusion protein recruits the RNAP holoenzyme, leading to transcription activation. In the B2H system, the BD is not directly fused to the AD; instead, their interaction is mediated by a protein-protein interaction (e.g., GAL11P-GAL4 interaction). To modify such a system for use in the methods, dCpf1 can be fused to a first protein sequence that provides for protein-protein interaction (e.g., the yeast GAL11P and/or GAL4 protein) and RNAa can be fused to a second protein sequence that completes the protein-protein interaction (e.g., GAL4 if GALl 1P is fused to dCpf1, GALl 1P if GAL4 is fused to dCpf1, etc.). The binding affinity between GAL11P and GAL4 increases the efficiency of binding and transcription firing rate.

A non-limiting example of a method using a dCpf1 fusion protein to increase transcription in eukaryotes includes fusion of dCpf1 to an activation domain (AD) (e.g., GAL4, herpesvirus activation protein VP16 or VP64, human nuclear factor NF-κB p65 subunit, etc.). To render the system inducible, expression of the dCpf1 fusion protein can be controlled by an inducible promoter (e.g., Tet-ON, Tet-OFF, etc.). The guide RNA can be design to target known transcription response elements (e.g., promoters, enhancers, etc.), known upstream activating sequences (UAS), sequences of unknown or known function that are suspected of being able to control expression of the target DNA, etc.

Additional Fusion Partners

Non-limiting examples of fusion partners to accomplish increased or decreased transcription include, but are not limited to, transcription activator and transcription repressor domains (e.g., the Kriippel associated box (KRAB or SKD); the Mad mSIN3 interaction domain (SID); the ERF repressor domain (ERD), etc.). In some such cases, the dCpf1 fusion protein is targeted by the guide RNA to a specific location (i.e., sequence) in the target DNA and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some embodiments, the heterologous sequence can be fused to the C-terminus of the dCpf1 polypeptide. In some embodiments, the heterologous sequence can be fused to the N-terminus of the dCpf1 polypeptide. In some embodiments, the heterologous sequence can be fused to an internal portion (i.e., a portion other than the N- or C-terminus) of the dCpf1 polypeptide.

The biological effects of a method using a dCpf1 fusion protein can be detected by any convenient method (e.g., gene expression assays; chromatin-based assays, e.g., Chromatin immunoPrecipitation (ChiP), Chromatin in vivo Assay (CiA), etc.).

In some cases, a method involves use of two or more different guide RNAs. For example, two different guide RNAs can be used in a single host cell, where the two different guide RNAs target two different target sequences in the same target nucleic acid.

Thus, for example, a transcriptional modulation method can further comprise introducing into the host cell a second guide RNA, or a nucleic acid comprising a nucleotide sequence encoding the second guide RNA, where the second guide RNA comprises: i) a first segment comprising a nucleotide sequence that is complementary to a second target sequence in the target DNA; ii) a second segment that interacts with the site-directed polypeptide; and iii) a transcriptional terminator. In some cases, use of two different guide RNAs targeting two different targeting sequences in the same target nucleic acid provides for increased modulation (e.g., reduction or increase) in transcription of the target nucleic acid.

As another example, two different guide RNAs can be used in a single host cell, where the two different guide RNAs target two different target nucleic acids. Thus, for example, a transcriptional modulation method can further comprise introducing into the host cell a second guide RNA, or a nucleic acid comprising a nucleotide sequence encoding the second guide RNA, where the second guide RNA comprises: i) a first segment comprising a nucleotide sequence that is complementary to a target sequence in at least a second target DNA; ii) a second segment that interacts with the site-directed polypeptide; and iii) a transcriptional terminator.

In some embodiments, a nucleic acid (e.g., a guide RNA, e.g., a single-molecule guide RNA; a donor polynucleotide; a nucleic acid encoding a site-directed modifying polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m⁷G)); a 3′ polyadenylated tail (i.e., a 3′ poly(A) tail); a riboswitch sequence or an aptamer sequence (e.g., to allow for regulated stability and/or regulated accessibility by proteins and/or protein complexes); a terminator sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

DNA-Targeting Segment

The DNA-targeting segment (or “DNA-targeting sequence”) of a guide RNA comprises a nucleotide sequence that is complementary to a specific sequence within a target DNA (the complementary strand of the target DNA).

In other words, the DNA-targeting segment of a guide RNA interacts with a target DNA in a sequence-specific manner via hybridization (i.e., base pairing). As such, the nucleotide sequence of the DNA-targeting segment may vary and determines the location within the target DNA that the guide RNA and the target DNA will interact. The DNA-targeting segment of a guide RNA can be modified (e.g., by genetic engineering) to hybridize to any desired sequence within a target DNA.

Stability Control Sequence (e.g., Transcriptional Terminator Segment)

A stability control sequence influences the stability of an RNA (e.g., a guide RNA,). One example of a suitable stability control sequence is a transcriptional terminator segment (i.e., a transcription termination sequence). A transcriptional terminator segment of a guide RNA can have a total length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the transcriptional terminator segment can have a length of from about 15 nucleotides (nt) to about 80 nt, from about 15 nt to about 50 nt, from about 15 nt to about 40 nt, from about 15 nt to about 30 nt or from about 15 nt to about 25 nt.

In some cases, the transcription termination sequence is one that is functional in a eukaryotic cell. In some cases, the transcription termination sequence is one that is functional in a prokaryotic cell.

Nucleotide sequences that can be included in a stability control sequence (e.g., transcriptional termination segment, or in any segment of the guide RNA to provide for increased stability) include, for example, a Rho-independent trp termination site.

Additional Sequences

In some embodiments, a guide RNA comprises at least one additional segment at either the 5′ or 3′ end. For example, a suitable additional segment can comprise a 5′ cap (e.g., a 7-methylguanylate cap (m⁷G)); a 3′ polyadenylated tail (i.e., a 3′ poly(A) tail); a riboswitch sequence (e.g., to allow for regulated stability and/or regulated accessibility by proteins and protein complexes); a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability; and combinations thereof.

Multiple Simultaneous Guide RNAs

In some embodiments, multiple guide RNAs are used simultaneously in the same cell to simultaneously modulate transcription at different locations on the same target DNA or on different target DNAs. In some embodiments, two or more guide RNAs target the same gene or transcript or locus. In some embodiments, two or more guide RNAs target different unrelated loci. In some embodiments, two or more guide RNAs target different, but related loci.

Because the guide RNAs are small and robust they can be simultaneously present on the same expression vector and can even be under the same transcriptional control if so desired. In some embodiments, two or more (e.g., 3 or more, 4 or more, 5 or more, 10 or more, 15 or more, 20 or more, 25 or more, 30 or more, 35 or more, 40 or more, 45 or more, or 50 or more) guide RNAs are simultaneously expressed in a target cell (from the same or different vectors). In some cases, multiple guide RNAs can be encoded in an array mimicking naturally occurring CRISPR arrays of targeter RNAs. The targeting segments are encoded as approximately 30 nucleotide long sequences (can be about 16 to about 100 nt) and are separated by CRISPR repeat sequences. The array may be introduced into a cell by DNAs encoding the RNAs or as RNAs.

To express multiple guide RNAs, an artificial RNA processing system mediated by the Csy4 endoribonuclease can be used. For example, multiple guide RNAs can be concatenated into a tandem array on a precursor transcript (e.g., expressed from a U6 promoter), and separated by Csy4-specific RNA sequence. Co-expressed Csy4 protein cleaves the precursor transcript into multiple guide RNAs. Advantages for using an RNA processing system include: first, there is no need to use multiple promoters; second, since all guide RNAs are processed from a precursor transcript, their concentrations are normalized for similar dCpf1-binding.

Csy4 is a small endoribonuclease (RNase) protein derived from bacteria Pseudomonas aeruginosa. Csy4 specifically recognizes a minimal 17-bp RNA hairpin, and exhibits rapid (<1 min) and highly efficient (>99.9%) RNA cleavage. Unlike most RNases, the cleaved RNA fragment remains stable and functionally active. The Csy4-based RNA cleavage can be repurposed into an artificial RNA processing system. In this system, the 17-bp RNA hairpins are inserted between multiple RNA fragments that are transcribed as a precursor transcript from a single promoter. Co-expression of Csy4 is effective in generating individual RNA fragments.

Site-Directed Polypeptide

As noted above, a guide RNA and a variant Cpf1 site-directed polypeptide form a complex. The guide RNA provides target specificity to the complex by comprising a nucleotide sequence that is complementary to a sequence of a target DNA. The variant Cpf1 site-directed polypeptide has reduced endodeoxyribonuclease activity. For example, a variant Cpf1 site-directed polypeptide suitable for use in a transcription modulation method of the present disclosure exhibits less than about 20%, less than about 15%, less than about 10%, less than about 5%, less than about 1%, or less than about 0.1%, of the endodeoxyribonuclease activity of a wild-type Cpf1 polypeptide, e.g., a wild-type Cpf1 polypeptide comprising an amino acid sequence set out in FIG. 1. In some embodiments, the variant Cpf1 site-directed polypeptide has substantially no detectable endodeoxyribonuclease activity. In some embodiments when a site-directed polypeptide has reduced catalytic activity, the polypeptide can still bind to target DNA in a site-specific manner (because it is still guided to a target DNA sequence by a guide RNA) as long as it retains the ability to interact with the guide RNA.

In some cases, a suitable variant Cpf1 site-directed polypeptide comprises an amino acid sequence having at least about 75%, at least about 80%, at least about 85%, at least about 90%, at least about 95%, at least about 99% or 100% amino acid sequence identity to FIG. 1.

In some cases, the variant Cpf1 site-directed polypeptide is a nickase that can cleave the complementary strand of the target DNA but has reduced ability to cleave the non-complementary strand of the target DNA.

In some cases, the variant Cpf1 site-directed polypeptide in a nickase that can cleave the non-complementary strand of the target DNA but has reduced ability to cleave the complementary strand of the target DNA.

In some cases, the variant Cpf1 site-directed polypeptide has a reduced ability to cleave both the complementary and the non-complementary strands of the target DNA. For example, alanine substitutions are contemplated.

In some cases, the variant Cpf1 site-directed polypeptide is a fusion polypeptide (a “variant Cpf1 fusion polypeptide”), i.e., a fusion polypeptide comprising: i) a variant Cpf1 site-directed polypeptide; and ii) a covalently linked heterologous polypeptide (also referred to as a “fusion partner”).

The heterologous polypeptide may exhibit an activity (e.g., enzymatic activity) that will also be exhibited by the variant Cpf1 fusion polypeptide (e.g., methyltransferase activity, acetyltransferase activity, kinase activity, ubiquitinating activity, etc.). A heterologous nucleic acid sequence may be linked to another nucleic acid sequence (e.g., by genetic engineering) to generate a chimeric nucleotide sequence encoding a chimeric polypeptide. In some embodiments, a variant Cpf1 fusion polypeptide is generated by fusing a variant Cpf1 polypeptide with a heterologous sequence that provides for subcellular localization (i.e., the heterologous sequence is a subcellular localization sequence, e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some embodiments, the heterologous sequence can provide for increased or decreased stability (i.e., the heterologous sequence is a stability control peptide, e.g., a degron, which in some cases is controllable (e.g., a temperature sensitive or drug controllable degron sequence, see below). In some embodiments, the heterologous sequence can provide for increased or decreased transcription from the target DNA (i.e., the heterologous sequence is a transcription modulation sequence, e.g., a transcription factor/activator or a fragment thereof, a protein or fragment thereof that recruits a transcription factor/activator, a transcription repressor or a fragment thereof, a protein or fragment thereof that recruits a transcription repressor, a small molecule/drug-responsive transcription regulator, etc.). In some embodiments, the heterologous sequence can provide a binding domain (i.e., the heterologous sequence is a protein binding sequence, e.g., to provide the ability of a chimeric dCpf1 polypeptide to bind to another protein of interest, e.g., a DNA or histone modifying protein, a transcription factor or transcription repressor, a recruiting protein, etc.).

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant Cpf1 polypeptide with controllable stability such that the variant Cpf1 polypeptide can be turned “on” (i.e., stable) or “off” (i.e., unstable, degraded) depending on the desired conditions. For example, if the degron is a temperature sensitive degron, the variant Cpf1 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off”, degraded) above the threshold temperature. As another example, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-i, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well characterized and tested in both cells and animals. Thus, fusing Cpf1 to a degron sequence produces a “tunable” and “inducible” Cpf1 polypeptide. Any of the fusion partners described herein can be used in any desirable combination. As one non-limiting example to illustrate this point, a Cpf1 fusion protein can comprise a YFP sequence for detection, a degron sequence for stability, and transcription activator sequence to increase transcription of the target DNA. Furthermore, the number of fusion partners that can be used in a Cpf1 fusion protein is unlimited. In some cases, a Cpf1 fusion protein comprises one or more (e.g., two or more, three or more, four or more, or five or more) heterologous sequences.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, any of which can be directed at modifying the DNA directly (e.g., methylation of DNA) or at modifying a DNA-associated polypeptide (e.g., a histone or DNA binding protein). Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pil 1/Aby 1, etc.).

In some embodiments, a site-directed modifying polypeptide can be codon-optimized. This type of optimization is known in the art and entails the mutation of foreign-derived DNA to mimic the codon preferences of the intended host organism or cell while encoding the same protein. Thus, the codons are changed, but the encoded protein remains unchanged. For example, if the intended target cell were a human cell, a human codon-optimized dCpf1 (or dCpf1 variant) would be a suitable site-directed modifying polypeptide. As another non-limiting example, if the intended host cell were a mouse cell, than a mouse codon-optimized Cpf1 (or variant, e.g., enzymatically inactive variant) would be a suitable Cpf1 site-directed polypeptide. While codon optimization is not required, it is acceptable and may be preferable in certain cases.

Polyadenylation signals can also be chosen to optimize expression in the intended host.

Host Cells

A method of the present disclosure to modulate transcription may be employed to induce transcriptional modulation in mitotic or post-mitotic cells in vivo and/or ex vivo and/or in vitro. Because the guide RNA provides specificity by hybridizing to target DNA, a mitotic and/or post-mitotic cell can be any of a variety of host cell, where suitable host cells include, but are not limited to, a bacterial cell; an archaeal cell; a single-celled eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell; an animal cell; a cell from an invertebrate animal (e.g., an insect, a cnidarian, an echinoderm, a nematode, etc.); a eukaryotic parasite (e.g., a malarial parasite, e.g., Plasmodium fakiparum; a helminth; etc.); a cell from a vertebrate animal (e.g., fish, amphibian, reptile, bird, mammal); a mammalian cell, e.g., a rodent cell, a human cell, a non-human primate cell, etc. Suitable host cells include naturally occurring cells; genetically modified cells (e.g., cells genetically modified in a laboratory, e.g., by the “hand of man”); and cells manipulated in vitro in any way. In some cases, a host cell is isolated.

Any type of cell may be of interest (e.g., a stem cell, e.g., an embryonic stem (ES) cell, an induced pluripotent stem (iPS) cell, a germ cell; a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures include cultures that may have been passaged 0 times, 1 time, 2 times, 4 times, 5 times, 10 times, or 15 times, but not enough times go through the crisis stage. Primary cell lines can be are maintained for fewer than 10 passages in vitro. Target cells are in many embodiments unicellular organisms, or are grown in culture.

If the cells are primary cells, such cells may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, e.g., from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% dimethyl sulfoxide (DMSO), 50% serum, 40% buffered medium, or some other such solution as is commonly used in the art to preserve cells at such freezing temperatures, and thawed in a manner as commonly known in the art for thawing frozen cultured cells.

Introducing Nucleic Acid into a Host Cell

A guide RNA, or a nucleic acid comprising a nucleotide sequence encoding same, can be introduced into a host cell by any of a variety of well-known methods. Similarly, where a method involves introducing into a host cell a nucleic acid comprising a nucleotide sequence encoding a variant Cpf1 site-directed polypeptide, such a nucleic acid can be introduced into a host cell by any of a variety of well-known methods.

Methods of introducing a nucleic acid into a host cell are known in the art, and any known method can be used to introduce a nucleic acid (e.g., an expression construct) into a stem cell or progenitor cell. Suitable methods include, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

Nucleic Acids

The present disclosure provides an isolated nucleic acid comprising a nucleotide sequence encoding a guide RNA. In some cases, a nucleic acid also comprises a nucleotide sequence encoding a variant Cpf1 site-directed polypeptide.

In some embodiments, a method involves introducing into a host cell (or a population of host cells) one or more nucleic acids comprising nucleotide sequences encoding a guide RNA and/or a variant Cpf1 site-directed polypeptide. In some embodiments a cell comprising a target DNA is in vitro. In some embodiments a cell comprising a target DNA is in vivo. Suitable nucleic acids comprising nucleotide sequences encoding a guide RNA and/or a site-directed polypeptide include expression vectors, where an expression vector comprising a nucleotide sequence encoding a guide RNA and/or a site-directed polypeptide is a “recombinant expression vector.”

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc. Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683-690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding a guide RNA and/or a variant Cpf1 site-directed polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a guide RNA and/or a variant Cpf1 site-directed polypeptide is operably linked to multiple control elements that allow expression of the nucleotide sequence encoding a guide RNA and/or a variant Cpf1 site-directed polypeptide in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca²⁺-calmodulin-dependent protein kinase II-alpha (CamKlla) promoter (see, e.g., Mayford et al. (1996) Proc. Natl. Acad. Sci. USA 93:13250; and Casanova et al. (2001) Genesis 31:37); a CMV enhancer/platelet-derived growth factor-0 promoter (see, e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyilrek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

Libraries

The present disclosure provides a library of guide RNAs. The present disclosure provides a library of nucleic acids comprising nucleotides encoding guide RNAs. A library of nucleic acids comprising nucleotides encoding guide RNAs can comprises a library of recombinant expression vectors comprising nucleotides encoding the guide RNAs.

A library can comprise from about 10 individual members to about 10¹³ individual members; e.g., a library can comprise from about 10 individual members to about 10² individual members, from about 10² individual members to about 10³ individual members, from about 10³ individual members to about 10⁵ individual members, from about 10⁵ individual members to about 10⁷ individual members, from about 10⁷ individual members to about 10⁹ individual members, or from about 10⁹ individual members to about 10¹² individual members.

An “individual member” of a library differs from other members of the library in the nucleotide sequence of the DNA targeting segment of the guide RNA. Thus, e.g., each individual member of a library can comprise the same or substantially the same nucleotide sequence of the protein-binding segment as all other members of the library; and can comprise the same or substantially the same nucleotide sequence of the transcriptional termination segment as all other members of the library; but differs from other members of the library in the nucleotide sequence of the DNA targeting segment of the guide RNA. In this way, the library can comprise members that bind to different target nucleic acids.

Uses

A method for modulating transcription according to the present disclosure finds use in a variety of applications, which are also provided. Applications include research applications; diagnostic applications; industrial applications; and treatment applications.

Research applications include, e.g., determining the effect of reducing or increasing transcription of a target nucleic acid on, e.g., development, metabolism, expression of a downstream gene, and the like.

High through-put genomic analysis can be carried out using a transcription modulation method, in which only the DNA-targeting segment of the guide RNA needs to be varied, while the protein-binding segment and the transcription termination segment can (in some cases) be held constant. A library (e.g., a library) comprising a plurality of nucleic acids used in the genomic analysis would include: a promoter operably linked to a guide RNA-encoding nucleotide sequence, where each nucleic acid would include a common protein-binding segment, a different DNA-targeting segment, and a common transcription termination segment. A chip could contain over 5×10⁴ unique guide RNAs. Applications would include large-scale phenotyping, gene-to-function mapping, and meta-genomic analysis.

The methods disclosed herein find use in the field of metabolic engineering. Because transcription levels can be efficiently and predictably controlled by designing an appropriate guide RNA, as disclosed herein, the activity of metabolic pathways (e.g., biosynthetic pathways) can be precisely controlled and tuned by controlling the level of specific enzymes (e.g., via increased or decreased transcription) within a metabolic pathway of interest. Metabolic pathways of interest include those used for chemical (fine chemicals, fuel, antibiotics, toxins, agonists, antagonists, etc.) and/or drug production.

Biosynthetic pathways of interest include but are not limited to (1) the mevalonate pathway (e.g., HMG-CoA reductase pathway) (converts acetyl-CoA to dimethylallyl pyrophosphate (DMAPP) and isopentenyl pyrophosphate (IPP), which are used for the biosynthesis of a wide variety of biomolecules including terpenoids/isoprenoids), (2) the non-mevalonate pathway (i.e., the “2-C-methyl-D-erythritol 4-phosphate/1-deoxy-D-xylulose 5-phosphate pathway” or “MEP/DOXP pathway” or “DXP pathway”)(also produces DMAPP and IPP, instead by converting pyruvate and glyceraldehyde 3-phosphate into DMAPP and IPP via an alternative pathway to the mevalonate pathway), (3) the polyketide synthesis pathway (produces a variety of polyketides via a variety of polyketide synthase enzymes. Polyketides include naturally occurring small molecules used for chemotherapy (e. g., tetracyclin, and macrolides) and industrially important polyketides include rapamycin (immunosuppressant), erythromycin (antibiotic), lovastatin (anticholesterol drug), and epothilone B (anticancer drug)), (4) fatty acid synthesis pathways, (5) the DAHP (3-deoxy-D-arabino-heptulosonate 7-phosphate) synthesis pathway, (6) pathways that produce potential biofuels (such as short-chain alcohols and alkane, fatty acid methyl esters and fatty alcohols, isoprenoids, etc.), etc.

Networks and Cascades

The methods disclosed herein can be used to design integrated networks (i.e., a cascade or cascades) of control. For example, a guide RNA/variant Cpf1 site-directed polypeptide may be used to control (i.e., modulate, e.g., increase, decrease) the expression of another DNA-targeting RNA or another variant Cpf1 site-directed polypeptide. For example, a first guide RNA may be designed to target the modulation of transcription of a second chimeric dCpf1 polypeptide with a function that is different than the first variant Cpf1 site-directed polypeptide (e.g., methyltransferase activity, demethylase activity, acetyltansferase activity, deacetylase activity, etc.). In addition, because different dCpf1 proteins (e.g., derived from different species) may require a different Cpf1 handle (i.e., protein binding segment), the second chimeric dCpf1 polypeptide can be derived from a different species than the first dCpf1 polypeptide above. Thus, in some cases, the second chimeric dCpf1 polypeptide can be selected such that it may not interact with the first guide RNA. In other cases, the second chimeric dCpf1 polypeptide can be selected such that it does interact with the first guide RNA. In some such cases, the activities of the two (or more) dCpf1 proteins may compete (e.g., if the polypeptides have opposing activities) or may synergize (e.g., if the polypeptides have similar or synergistic activities). Likewise, as noted above, any of the complexes (i.e., guide RNA/dCpf1 polypeptide) in the network can be designed to control other guide RNAs or dCpf1 polypeptides. Because a guide RNA and variant Cpf1 site-directed polypeptide can be targeted to any desired DNA sequence, the methods described herein can be used to control and regulate the expression of any desired target. The integrated networks (i.e., cascades of interactions) that can be designed range from very simple to very complex, and are without limit.

In a network wherein two or more components (e.g., guide RNAs or dCpf1 polypeptides) are each under regulatory control of another guide RNA/dCpf1 polypeptide complex, the level of expression of one component of the network may affect the level of expression (e.g., may increase or decrease the expression) of another component of the network. Through this mechanism, the expression of one component may affect the expression of a different component in the same network, and the network may include a mix of components that increase the expression of other components, as well as components that decrease the expression of other components. As would be readily understood by one of skill in the art, the above examples whereby the level of expression of one component may affect the level of expression of one or more different component(s) are for illustrative purposes, and are not limiting. An additional layer of complexity may be optionally introduced into a network when one or more components are modified (as described above) to be manipulable (i.e., under experimental control, e.g., temperature control; drug control, i.e., drug inducible control; light control; etc.).

As one non-limiting example, a first guide RNA can bind to the promoter of a second guide RNA, which controls the expression of a target therapeutic/metabolic gene. In such a case, conditional expression of the first guide RNA indirectly activates the therapeutic/metabolic gene. RNA cascades of this type are useful, for example, for easily converting a repressor into an activator, and can be used to control the logics or dynamics of expression of a target gene.

A transcription modulation method can also be used for drug discovery and target validation.

Various aspects of the invention make use of the following materials and methods and are illustrated by the following non-limiting examples.

### EXAMPLES

Cpf1 is a Single CRISPR-Associated Protein that Carries Both RNA- and DNA-Cleaving Activities

The intracellular human pathogen Francisella novicida U112 was previously analysed by small RNA (sRNA) sequencing. Identified were sRNAs expressed from two CRISPR-Cas loci (FIG. 5). In addition to the Type II-B locus, sRNAs expressed from a CRISPR-Cas locus that resembled the minimal architecture of Type II systems were detected, but lacked a cas9 gene. FTN_1397 located upstream of the cas1-cas2-cas4 genes was identified as a cas gene encoding a protein distinct in sequence from known Cas proteins, and was later named cpf1 (cas gene of Pasteurella, Francisella). This system was recently classified as a Type V-A system belonging to class 2 of the CRISPR-Cas systems. The Type V CRISPR array contained a series of 9 spacer sequences separated by 36-nt repeat sequences. The mature RNAs were composed of repeat sequence in 5′ and spacer sequence in 3′, similar to the repeat-spacer composition of Type I and III systems, but distinct from the spacer-repeat composition of Type II systems (FIG. 5). Similar to the Type I system, the repeat formed a hairpin structure located at the 3′ end of the repeat. Neither the presence of an anti-CRISPR repeat nor the expression of a tracrRNA homolog could be detected in the vicinity of the F. novicida Type V-A locus, indicating that Cpf1 uses a distinct mode of crRNA biogenesis compared to the already described mechanisms. Possible transcription of shorter pre-crRNA fragments from within the CRISPR array were undetectable, as already reported for a Type II-C system.

Investigated next was whether Cpf1 might act as the single effector enzyme in pre-crRNA processing in Type V-A systems. Recombinant F. novicida Cpf1 protein was overexpressed and purified. Size-exclusion chromatography was performed to determine the oligomeric state of the protein. In contrast to the recently reported formation of Cpf1 dimers in solution, analysis of our data revealed a molecular weight of 187 kDa (FIG. 6), indicating that Cpf1 is a monomer. In vitro cleavage assays show that Cpf1 processed RNA consisting of a full-length repeat-spacer, yielding a 19 nt repeat fragment and a 50 nt repeat-spacer crRNA (FIG. 1). Only RNAs with full-length repeat sequences were processed, indicating that the RNA cleavage activity of Cpf1 is repeat-dependent (FIG. 6). Northern Blot analysis using an inducible E. coli heterologous system also demonstrated processing of a pre-crRNA upon Cpf1 expression (FIG. 8), resulting in the expected RNA fragments. Cpf1 cleaved pre-crRNA 4 nucleotides upstream of the stem-loop (FIG. 2). This was reminiscent to many Cas6 enzymes and Cas5d, which recognize the hairpin of their respective repeats. Cpf1, however, did not cleave directly at the base of the stem-loop, suggesting that the structure is not the only requirement for processing of pre-crRNA. RNAs with mutations that yield either an altered repeat sequence keeping the stem-loop structure or an unstructured repeat were designed. In contrast to wild type RNA substrate containing an intact repeat, none of the mutated RNAs were cleaved by Cpf1 (FIG. 9), indicating that the repeat cleavage reaction is sequence and structure dependent.

To determine the ion-dependency of Cpf1 processing activity, a variety of divalent metal ions were tested in RNA cleavage assays. The activity of Cpf1 in pre-crRNA processing was best when Mg²⁺ was added to the reaction (FIG. 10A). Supplementation with Ca²⁺, Mn²⁺ and Co²⁺ also mediated cleavage, however not to the level of specificity observed with Mg²⁺. This was in contrast to the ion-independent reaction of Cas6 enzymes (Types I and III) or Cas5d (Type I-C). Thus, this study highlights a novel crRNA biogenesis mechanism in which Cpf1 is a metal-dependent endoribonuclease cleaving pre-crRNA in a sequence and structure specific manner. Bioinformatic analyses indicate that Type V-A may be an ancestral version of Type II systems and may have evolved from Type I systems through transposition events. The finding that Cpf1 functions as the endoribonuclease of Type V-A systems together with the repeat-spacer composition of mature crRNAs and the requirement for a hairpin structure provides evidence to support this hypothesis.

As part of a minimal CRISPR-Cas system, Cpf1 is likely responsible for DNA interference, similarly to Cas9. As reported recently by us and others, Cpf1 acts as a DNA endonuclease guided by crRNA to cleave dsDNA site-specifically. The DNA cleavage specificity of Cpf1 on plasmid and oligonucleotides containing protospacer 5 using crRNA containing either spacer 4 or spacer 5 was investigated. Only crRNA complementary to the target mediated Cpf1 DNA cleavage (FIGS. 3A and 3B). To further analyse the RNA requirements for this activity, several RNAs containing various structures were constructed (RNAs 1-8, FIG. 11). Only RNAs with an intact stem-loop were able to mediate Cpf1 DNA cleavage activity (RNA 3-7, FIGS. 11A and 11B). Surprisingly, the RNA with a spacer-repeat arrangement also mediated cleavage activity, albeit with less efficiency than the wild type. The RNA processing activity of Cpf1 was highly dependent on the repeat sequence (FIG. 9), however a similar RNA resulted in residual DNA cleavage activity (RNA 7, FIG. 11). This might have been due to the 3′ end nucleotide of the repeat, which was not mutated and was recently reported to be critical. Because Cpf1 can process pre-crRNA, it is not surprising that RNAs with the full-length repeat-spacer (RNA4 and RNA6, FIG. 9) mediated similar cleavage activities as the mature crRNA form. The RNA containing the full-length repeat-spacer resulted in most efficient DNA binding and nuclease activity of Cpf1 (compare RNA4 to RNA3 and RNA6, FIG. 12A and FIG. 11B). The processed form of crRNA (RNA3, FIG. 11) was constructed based on sRNA sequencing results (FIG. 5) before knowing the exact RNA processing of Cpf1, which resulted in a 2 nt shorter 5′ end (FIG. 2). Processing of RNA6 (repeat-spacer-repeat, FIG. 11) resulted in a RNA containing processed repeat-full-length spacer-I19 nt repeat. It is likely that both RNAs did not lead to the ideal conformational changes of Cpf1 upon their binding to mediate full DNA targeting activity. Best binding activities were achieved when RNA4 was used (FIG. 12A). Therefore, RNA4 was chosen for further characterization.

A split RuvC motif was reported to be responsible for DNA cleavage activity of Cpf1. The metal ion dependency of DNA cleavage was investigated. Remarkably, it was observed that in addition to Mg²⁺ and Mn²⁺, which were shown to mediate activity in Cas9, Cpf1 cleaved DNA in the presence of Ca²⁺ (FIG. 10B). To investigate potential differences in cleavage with Mg²+ or Ca²+, DNA cleavage reactions in the presence of either of these ions (FIG. 3, FIG. 13) were performed. In contrast to a recent publication showing that the HNH motif of Cas9 from Neisseria meningitidis is Ca²+ dependent, significant differences in target or non-target strand cleavage efficiency of Cpf1 in the presence of Ca²⁺or Mg²⁺ (FIG. 3B; FIG. 13B) were not observed. This indicated the presence of only one catalytic motif in Cpf1 that is responsible for cleaving both DNA strands, and can coordinate Mg²⁺ as well as Ca²⁺ ions.

Cleavage reactions using oligonucleotide duplexes with either radiolabeled target or non-target strand generated products of different sizes (FIG. 3B, FIG. 13B). This observation was confirmed by sequencing of plasmid cleavage products (FIGS. 13A and 13C), that demonstrated a staggered cut by Cpf1 producing a 5 nt 5′ overhang, as reported recently.

Aligning the two predicted protospacer sequences of the F. novicida U112 type V-A CRISPR-Cas revealed a conserved 5′-TTA-3′ sequence located on the non-target strand upstream of the protospacer. To verify the potential PAM, protospacer 5 was cloned without its flanking region yielding a 5′-CTG-3′ sequence. Both plasmids were cleaved equally well by Cpf1, indicating that the second position in this sequence is critical (FIG. 3d, FIG. 14d). Mutagenesis of all three nucleotides followed by DNA cleavage analysis shows that Cpf1 recognizes a PAM, defined as 5′-YTN-3′, upstream of the crRNA-complementary DNA sequence on the non-target strand. This result expands on the already reported 5′-TTN-3′ PAM reported by Zetsche et al. (Cell, 2015, 163:759-771). To analyze strand specificity of PAM recognition, oligonucleotide substrates with either AAN or TTN on both strands were designed. These substrates were not cleaved by Cpf1, indicating that the PAM needs to be double-stranded and is probably recognized on both strands (FIG. 3D, lower panel). Cpf1 has a seed sequence of eight nucleotides proximal to the PAM. During interference of Type I and II systems the first 8-10 nt of the protospacer are crucial to enable the formation of a stable R-loop. This sequence is called seed sequence. Type II cleavage occurs 3 bp upstream of the PAM within the protospacer. In contrast, the PAM and cleavage site of Cpf1 lie on opposite sides of the protospacer. To analyze the length of the seed sequence, plasmids having single mismatches between spacer and protospacer along the target sequence were constructed. Cpf1 is sensitive to mismatches within the first 8 nucleotides on the PAM proximal side, while four consecutive mismatches are not tolerated. Furthermore, Cpf1 shows sensitivity to mismatches around the cleavage site (position 1-4 on the PAM distal site), however to a lesser extent. These results are in discrepancy to already published data showing a seed sequence of only 3-5 nucleotides PAM proximal, indicating that there might be other factors influencing the specificity, like the base content of the target sequence. These results indicate that Cpf1, similar to Cas9, first recognizes the PAM and then tests crRNA complementarity to the DNA target. Mismatches around the target site might disturb correct positioning of the catalytic residues and therefore reduce cleavage activity.

Cpf1 comprises a dual activity of RNA and DNA cleavage, and uses distinct active domains for each nuclease reaction. To determine the active motifs, mutagenesis of conserved residues along the Cpf1 amino acid sequence was performed. Alanine substitution of residues H843, K852, K869 and F873 had no effect on DNA cleavage activity but showed decreased in vitro RNA cleavage activity. Mutagenesis of D917, E1006 and D1255 in the split RuvC motif resulted in loss of DNA cleavage activity, but did not influence the RNA processing activity of Cpf1, nor did it affect binding affinity to the DNA target. See FIGS. 4D and 13B. To determine the active motifs, mutagenesis of conserved residues along the Cpf1 amino acid sequence were performed. FIG. 4D summarizes mutated residues, which impact one of the two catalytic activities. Alanine substitution of residues H843, K852, K869 and F873 had no effect on DNA cleavage activity (FIG. 4A, upper panel), but showed decreased in vitro RNA cleavage activity (FIG. 4A, middle panel). To further confirm their involvement in RNA processing in vivo, a heterologous E. coli assay co-expressing pre-crRNA (repeat-spacer-repeat) and Cpf1 or a variant thereof was set up. Northern Blot analysis was done with total RNA extracted after induced expression (FIG. 4A, lower panel). It seems that in the presence of Cpf1, crRNA was protected from degradation and therefore more abundant. Expression of Cpf1_wt results in the production of a distinct band of around 65 nt, which corresponds to a mature crRNA formed by two cleavage events within the repeats. In presence of Cpf1_H843A, this band was not present; however, two additional longer transcripts appeared due to a changed processing by this mutant, already seen in vitro (FIG. 4A, middle panel). Mutants K852A and K869A also showed the production of the 65 nt fragment, although with less intensity compared to the wild type and in addition to the two products of longer sizes. In vitro, these mutants showed almost no RNA processing. RNA-binding experiments with Cpf1 (K852A) and Cpf1 (K869A) (FIG. 12C) indicated a slightly higher affinity for RNA than wild-type Cpf1, which may explain the cleavage products observed in vivo. The residual activity of these Cpf1 mutants produces processed RNA, which is likely to be bound tighter to the protein and therefore better protected from degradation. Cpf1 (F873A) had reduced RNA cleavage activity in vitro, which could not be detected in vivo. Mutation of the aforementioned residues did not negatively affect RNA binding (FIG. 12C), indicating that the identified residues of Cpf1 are potentially responsible for RNA cleavage. Analysis of the co-crystal structure of Lachnospiraceae bacterium Cpf1 revealed that the identified residues are located in close proximity to the 5′ of the processed crRNA (Dong et al. (2016) Nature, 532(7600):522-6). Mutagenesis of D917, E1 006 and D1255 in the split RuvC motif resulted in loss of DNA cleavage activity (FIG. 4D, upper panel) (see also Zetsche et al. (2015) Cell, 163:759-771), but did not influence the RNA processing activity of Cpf1 (FIG. 4B, lower panel), nor did it affect binding affinity to the DNA target (FIG. 12B).

Cpf1 mutants display metal ion dependent differences in DNA cleavage. While screening for active site residues, significant differences in DNA cleavage for some mutants was observed, dependent on the metal ion present in the reaction. Mutants E920A, Y1024A, and D1227A showed no DNA cleavage in the presence of Ca²⁺, but wild type activity when Mg²⁺ was present. Mutating residue E1028 also leads to loss of Ca²⁺ dependent cleavage and additionally decreases cleavage of the non-target strand in the presence of Mg²⁺, indicative of an involvement in non-target strand cleavage. In contrast, mutation of residues H922 and Y925 resulted in drastically decreased cleavage of the target strand in the presence of Ca²⁺. These mutants showed wild type levels of DNA cleavage activity in the presence of Mg²⁺. This suggests an involvement in Ca²⁺ coordination and target strand cleavage. Thus, Cpf1 can therefore be “ionically modulated” by altering the relative levels of calcium and/or magnesium to which the protein is exposed. Structural modifications can also be used to further modulate Cpf1. By inactivating the endonuclease activity of Cpf1 through mutations affecting the enzymatic activity, the protein can also be used to bind sequence-specifically without cleaving the DNA.

Two aspartates (D917, D1255) and one glutamate (E1006) form the catalytic site of Cpf1, which is in good agreement with other RuvC/RNaseH motifs. These kinds of catalytic motifs generally employ a two-metal-ion mechanism for DNA cleavage. Enzymes with a two-metal-ion mechanism are more stringent in the choice of the metal ion, with mostly a preference for Mg²⁺. In contrast, enzymes using a one-metal-ion mechanism for cleavage, like HNH nucleases, can be more flexible in their choice of metal ions. For example, Kpnl cleaves DNA with high fidelity in the presence of Ca²⁺, but more unspecifically in the presence of Mg²⁺. Cpf1 may also represent a new type of DNA-nuclease using two-metal-ion catalysis with the ability to utilize Mg²⁺ or Ca²⁺ ions.

Cpf1 is an enzyme with dual nucleolytic activity against RNA and DNA. Cpf1 is an enzyme that cleaves RNA in a highly sequence and structure dependent manner, and also performs specific DNA cleavage only in presence of the produced guide RNA. In context of CRISPR immunity, type V-A is the most efficient system described so far, utilizing only one enzyme, Cpf1, to process crRNA and to use this RNA to specifically target invading DNA. Cpf1 differs fundamentally from type II systems in that a complex of Cpf1 and a single RNA, the crRNA, can cleave DNA without the presence of a second RNA (such as the tracrRNA required in type II Cas9 systems). In context of CRISPR immunity, type V-A is the most efficient system described so far, utilizing only one enzyme, Cpf1, to process crRNA and use this RNA to specifically target invading DNA.

Materials and Methods

Small RNA Sequencing

Small RNA sequencing data of Francisella novicida U112 (Table 1) used in this study were obtained previously. Briefly, a cDNA library of Tobacco acid pyrophosphatase (TAP) (Epicentre)-treated RNAs of F. novicida U112 grown to mid-logarithmic phase was prepared using the ScriptMiner™ Small RNA-Seq Library Preparation Kit (Multiplex, Illumina@ compatible) and sequenced at the Campus Science Support Facilities GmbH (CSF) Next Generation Sequencing (NGS) Unit of the Vienna Biocenter. After adapter removal and quality trimming, the reads were mapped to the F. novicida U112 genome (GenBank: NC_008601, 48205 mapped reads) using Bowtie. The read coverage was calculated using BEDTools (Version 2.15.0.) and a normalized wiggle file was created and visualised using the Integrative Genomics Viewer (IGV) (www.broadinstitute.org/igv/).

Production and Purification of Recombinant Cpf1

The cpf1 (FTN_1397) gene was amplified from genomic DNA of F. novicida U112 and cloned into the expression vector pET-16b to facilitate expression of Cpf1 with an N-terminal 6×His-tag (Tables 2 and 3). For the production of the protein in Escherichia coli (NiCo21 (DE3)), the cells containing the overexpression plasmid were grown at 37° C. to reach an OD_{600 nm} of 0.6 to 0.8. The expression was induced by addition of 0.5 mM IPTG (isopropylthio-β-D-galactoside) and the cultures were further incubated overnight at 18° C. After harvesting, the cell pellet was resuspended in lysis buffer (20 mM HEPES [pH 7.5], 500 mM KCl, 25 mM imidazole, 0.1% triton X-100) followed by 6 min of sonication (0.5 s pulses) for cell disruption. The lysate was cleared by centrifugation (47800 g, 30 min, 4° C.) and the supernatant was applied to Ni-NTA-Sepharose resin in a drop column. After washing steps with 10 ml of lysis buffer followed by 10 ml wash buffer (20 mM HEPES [pH 7.5], 300 mM KCl, 25 mM imidazole), the protein was eluted with elution buffer (20 mM HEPES [pH 7.5], 150 mM KCl, 250 mM imidazole, 0.1 mM DTT, 1 mM EDTA). The eluates were analysed via SDS-PAGE followed by coomassie blue staining. Fractions containing Cpf1 were pooled for cation-exchange chromatography (HiTrap Heparin [GE-Healthcare]) using a FPLC Äkta-Purification system (GE-Healthcare) and Cpf1 was eluted with a linear gradient of potassium chloride (100-1000 mM KCl). Peak fractions were analysed by SDS-PAGE and coomassie blue staining. Cpf1 containing fractions were pooled and directly applied to an equilibrated (20 mM HEPES [pH 7.5], 150 mM KCl) prepgrade Superdex 200 size-exclusion column (GE-Healthcare) and purified via FPLC, followed by analysis by SDS-PAGE and coomassie blue staining. Molecular weight calibration of the column was performed using molecular weight markers as described in the manufacturer's protocol (Kit for Molecular Weights, Sigma-Aldrich). The protein was dialyzed against dialysis buffer (20 mM HEPES [pH 7.5], 150 mM KCl, 50% glycerol) and stored at −20° C. until use.

Site-Directed Mutagenesis of Cpf1

Oligonucleotides for the site-directed mutation of Cpf1 (Table 3) were designed using the QuickChange Primer Design tool of Agilent and produced by Sigma-Aldrich. Two individual PCRs were performed to obtain the desired mutation. Briefly, the vector containing wild type cpf1 was amplified in two reactions containing either the forward or reverse QuickChange primer. After an initial amplification, the two reactions were mixed and a second PCR was done. Following the PCR, the template plasmid was degraded with Dpnl (3 h, 37° C.) and transformed into chemically competent DH5-alpha cells. Plasmids were prepared using a plasmid Miniprep kit (Qiagen) according to the manufacturer's instructions. Successful mutagenesis was confirmed by sequencing (SeqLab).

Generation of RNAs Used in this Study

The small RNAs used in this study were generated by in vitro transcription using the AmpliScribe T7-Flash kit (Biozym) according to the manufacturer's protocol. In brief, oligonucleotides containing the desired sequence (Table 3) and a T7-promoter sequence were hybridized to an oligonucleotide containing the complementary T7-promoter sequence. The hybridization product was then used as template for the transcription reaction according to the AmpliScribe T7-Flash kit (Biozym). To obtain internally labeled RNAs, [α-³²P] ATP (5000 ci/mmol, Hartman Analytic) was added to the in vitro transcription reaction. In order to generate end labeled RNAs, the unlabeled transcripts were dephosphorylated with Fast-AP phosphatase (Fermentas) for 30 min at 37° C. followed by a purification using Illustra Microspin G-25 columns (GE-Healthcare). The dephosphorylated RNAs were then labeled using T4 polynucleotide kinase (Fermentas) and [γ-³²P] ATP (5000 ci/mmol) according to the manufacturer's instructions. Produced RNAs were separated using denaturing polyacrylamide gel electrophoresis (8 M urea; 1X TBE; 10% polyacrylamide). Subsequent to short exposure to an autoradiography screen (for radioactively labeled RNAs) or ethidium bromide (EtBr) staining (for unlabeled RNAs), the respective bands of the RNAs were excised. Elution of the RNAs was achieved by incubation of the gel pieces in 500 μL RNA elution buffer (250 mM NaOAc; 20 mM Tris/HCl [pH 7.5]; 1 mM EDTA [pH 8.0]; 0.25% SDS) and overnight incubation on ice. Following elution, RNA was precipitated with 2 Vol ethanol (EtOH 100%; ice cold) and 1/100 glycogen for 1 h at −20° C. Subsequent to washing with 70% EtOH, the air-dry pellets were resuspended in H₂O_{mq}.

In Vitro RNA Cleavage Assay

RNA cleavage assays using indicated concentrations of Cpf1 and various RNA substrates were conducted in KGB buffer (100 mM potassium glutamate, 25 mM Tris/acetate [pH 7.5], 500 μM 2-mercaptoethanol, 10 μg/ml BSA) supplemented with 10 mM MgCl₂ at 37° C. in a final volume of 10 μl. If not indicated otherwise, the reaction was stopped after 10 min by the addition of 2 μl proteinase K (20 mg/ml) following 10 min incubation at 37° C. to achieve protein degradation. After adding 2X loading dye (10 M urea, 1.5 mM EDTA [pH 8.0]), the samples were loaded on 12% denaturing polyacrylamide gels run in 1X TBE for 3 h at 12.5 V/cm. For the sequencing gels, the samples were precipitated prior to loading on 10% denaturing polyacrylamide gels. The gel electrophoresis was carried out at 40 W for 3.5 h. Visualization was achieved by phosphorimaging (Typhoon FLA 9000 Fuji).

In Vivo RNA Processing

To investigate in vivo RNA processing by Cpf1, a heterologous system was designed in E. coli. A DNA fragment encoding a crRNA containing a repeat-spacer-repeat structure under the control of a T7-promoter and T7-terminator was synthesized by Integrated DNA Technologies (IDT) and cloned into pACYC184 using HindIII and EagI yielding pEC1690. E. coli BL21(DE3) was co-transformed with this plasmid and the overexpression vector of wild type or mutant Cpf1. The empty expression vector pET-16b served as a negative control. The bacterial cells were grown in the presence or absence of 0.1 mM IPTG at 37° C. to reach early exponential phase (OD_{600 nm} of 0.4). RNA was extracted using TRIzol (Sigma-Aldrich) according to the manufacturer's protocol followed by Northern Blot analysis as described previously. In brief, RNA was separated on denaturing 10% polyacrylamide gels (8 M urea, 1X TBE) and transferred by semi-dry blotting on a nylon membrane (Hybond™ N+, GE Healthcare). Chemical crosslinking was done for 1 h at 60° C. with EDC (1-ethyl-3-(3-dimethylaminopropyl) carbodiimide hydrochloride). Oligonucleotides were radioactively labeled with [γ-³²P] ATP (5000 ci/mmol) and T4 polynucleotide kinase (Fermentas) as described above and purified using Illustra Microspin G-25 columns (GE healthcare). The hybridization of the probe was done in Rapid-hyb buffer (GE-Healthcare) by incubation overnight at 42° C. The radioactive signal was visualised using phosphorimaging.

Generation of DNA Substrates

To find the target cleavage site of Cpf1, spacer sequences of the F. novicida U112 Type V CRISPR array were analysed by BLAST. Potential targets for spacer 4 and spacer 5 were identified in F. novicida 3523, located in the intergenic region between CDS: AEE26308.1 and CDS: AEE26307.1 and in CDS: AEE26301.1, respectively. Target protospacer containing sequence complementary to spacer 5 including 42 bp up- and downstream was synthesized as double-stranded (ds) oligonucleotides having HindIII overhangs. Following hybridization of the oligonucleotides, the fragments were cloned into pUC19 using HindIII yielding plasmid pEC1664 (protospacer 5+flanking region). The same protospacer sequence without flanking regions was cloned into pUC19, yielding pEC1688 (protospacer 5). In order to identify the PAM, mutagenesis was performed by applying the described protocol for site-directed mutagenesis on pEC1688. Plasmid preparation was done using Miniprep kit (Qiagen) according to the manufacturer's instructions and DNA integrity was confirmed by sequencing (SeqLab). Oligonucleotides containing the protospacer (Table xx) were ordered at Sigma and hybridized prior radioactive labeling. Alternatively, a single stranded (ss) oligonucleotide was labeled and hybridized with the complementary non-labeled oligonucleotide. 5′ end labeling reactions were performed using [γ-³²P] ATP (5000 ci/mmol) and T4 polynucleotide kinase (Fermentas) according to the manufacturer's instructions. The labeled oligonucleotides were purified using Illustra Microspin G-25 columns (GE healthcare).

In Vitro DNA Cleavage Assay

Plasmid DNA cleavage assays were performed by pre-incubating 100 nM Cpf1 with 200 nM RNA in KGB supplemented with either 10 mM MgCl₂ or 10 mM CaCl₂ for 15 min at 37° C. 10 nM plasmid DNA were added to the reaction to yield a final volume of 10 μl and further incubated for 1 h at 37° C. Reactions were stopped by the addition of 1 μl proteinase K (20 mg/ml) and 5 min incubation at 37° C. Prior separation of the reaction, 3 μl of 5×DNA loading buffer (250 mM EDTA, 1.2% SDS, 25% glycerol, 0.01% bromophenol blue) were added and the samples were loaded on 0.8% agarose gels (1X TAE). Cleavage products were visualised by EtBr staining. In cleavage assays using radioactively labeled substrates, 5 nM of 5′ labeled ds oligonucleotides were added to the pre-formed complex of Cpf1 and RNA, and incubated at 37° C. for 1 h. After proteinase K treatment, 10 μl of 2X denaturing loading buffer (95% formamide, 0.025% SDS, 0.5 mM EDTA, 0.025% bromophenol blue) were added. Oligonucleotides of the size of the expected cleavage products were 5′ radiolabeled as described above and mixed with an equal volume of 2X denaturing loading buffer to serve as size marker. After 5 min incubation at 95° C., the samples were loaded on 12% denaturing polyacrylamide gels and run in 1X TBE for 70 min at 14 V/cm. Cleavage was visualised using phosphorimaging.

Electrophoretic Mobility Shift Assays (EMSAs)

Substrates for EMSAs were generated as described above. For binding reactions, Cpf1 was pre-incubated in binding buffer (200 mM Tris-HCl pH 7.4, 1 M KCl, 10 mM DTT, 50% glycerol) containing 2 molar excess of crRNA. After 15 minutes at 37° C., 1 nM labeled DNA substrate was added. The reaction was then carried out at 37° C. for 1 h before the samples were loaded on a native 5% polyacrylamide gel running in 0.5X TBE to separate protein-DNA complexes from unbound DNA. The gels were exposed on an autoradiography film overnight and visualised by phosphorimaging.

Multiple Sequence Alignment of Cpf1 Orthologues

Cpf1 orthologous sequences were derived by BLAST search of the NCBI database using Cpf1 of F. novicida U112 as a query. A multiple sequence alignment of 52 orthologous sequences was generated using MUSCLE. The alignment of nine of the sequences was visualised with Jalview.

The below tables describe the list of strains (Table 1), plasmids (Table 2) and oligonucleotides (Table 3) used in the study.

TABLE 1
Strains used in the study

| Strain | Relevant characteristics | Source |
|---|---|---|
| Francisella novicida | | |
| EC1041 | U112 (WT) | Anders Sjöstedt |
| Escherichia coli | | |
| RDN204 | TOP10; Host for cloning | Invitrogen |
| RDN226 | DH5α; Host for cloning | New England Biolabs |
| EC2212 | NiCo21 (DE3); Expression | New England Biolabs |
| | strain | |

TABLE 2
Plasmids used in the study

| | | |
|---|---|---|
| Plasmids | Relevant characteristics | Source |

Plasmids for in vitro protospacer study

| | | |
|---|---|---|
| pUC19 | | New England Biolabs |
| pEC1664 | pUC19ΩCpf1 (psp5) | This study |
| pEC1688 | pUC19Ωpsp5 | This study |
| pEC1693 | pUC19Ωpsp5_PAM A2C | This study |
| pEC1703 | pUC19Ωpsp5_A1C | This study |
| pEC1704 | pUC19Ωpsp5_G2T | This study |
| pEC1705 | pUC19Ωpsp5_A3C | This study |
| pEC1706 | pUC19Ωpsp5_T4G | This study |
| pEC1707 | pUC19Ωpsp5_A5C | This study |
| pEC1708 | pUC19Ωpsp5_G6T | This study |
| pEC1709 | pUC19Ωpsp5_A7C | This study |
| pEC1710 | pUC19Ωpsp5_A8C | This study |
| pEC1711 | pUC19Ωpsp5_T9G | This study |
| pEC1712 | pUC19Ωpsp5_T10G | This study |
| pEC1713 | pUC19Ωpsp5_A11C | This study |
| pEC1714 | pUC19Ωpsp5_C12A | This study |
| pEC1715 | pUC19Ωpsp5_C13A | This study |
| pEC1716 | pUC19Ωpsp5_T14G | This study |
| pEC1717 | pUC19Ωpsp5_T15G | This study |
| pEC1718 | pUC19Ωpsp5_T16G | This study |
| pEC1719 | pUC19Ωpsp5_T17G | This study |
| pEC1720 | pUC19Ωpsp5_A18C | This study |
| pEC1721 | pUC19Ωpsp5_A19C | This study |
| pEC1722 | pUC19Ωpsp5_T20G | This study |
| pEC1723 | pUC19Ωpsp5_C21A | This study |
| pEC1724 | pUC19Ωpsp5_T22G | This study |
| pEC1725 | pUC19Ωpsp5_mut1 | This study |
| pEC1726 | pUC19Ωpsp5_mut2 | This study |
| pEC1731 | pUC19Ωpsp5_PAM_G3T | This study |
| pEC1734 | pUC19Ωpsp5_PAM_A2C, GG3, | This study |
| | 7TT | |
| pEC1735 | pUC19Ωpsp5_PAM_A2G | This study |

Plasmids for Cpf1 overexpression

| | | |
|---|---|---|
| pEC621 | pEC225 + NotI, SacI, SalI | |
| pEC1611 | pEC621Ωcpf1 | This study |
| pEC1776 | pEC621Ωcpf1 (H843A) | This study |
| pEC1777 | pEC621Ωcpf1 (K852A) | This study |
| pEC1778 | pEC621Ωcpf1 (K869A) | This study |
| pEC1779 | pEC621Ωcpf1 (F873A) | This study |
| pEC1782 | pEC621Ωcpf1 (D917A) | This study |
| pEC1783 | pEC621Ωcpf1 (E920A) | This study |
| pEC1784 | pEC621Ωcpf1 (H922A) | This study |
| pEC1785 | pEC621Ωcpf1 (Y925A) | This study |
| pEC1788 | pEC621Ωcpf1 (E1006A) | This study |
| pEC1790 | pEC621Ωcpf1 (Y1024A) | This study |
| pEC1791 | pEC621Ωcpf1 (E1028A) | This study |
| pEC1796 | pEC621Ωcpf1 (D1227A) | This study |
| pEC1799 | pEC621Ωcpf1 (D1255A) | This study |

Plasmids for Northern blot analysis of pre-crRNA processing

| | | |
|---|---|---|
| pACYC184 | | New England Biolabs |
| pEC1690 | pACYCΩsgRNA2 | This study |
| pEC575 | pCDF-1b | Novagen |
| pEC1701 | pCDFΩcpf1 | This study |

TABLE 3
Oligonucleotides used in the study

| | | | | |
|---|---|---|---|---|
| | Primer | | | |
| Purpose | code | Sequence 5′-3′ | F/R | Usage |

Oligonucleotides for in vitro protospacer studies

| | | | | |
|---|---|---|---|---|
| pEC1664 | OLEC6213 | AGCTGTAGCAAATATTAATCATATAGAAGAAAGCTCAGAT | F | Cloning |
| | | CTCAACAAGATAGAATTACCTTTTAATCTTAAATTATTATA | | |
| | | TCCAGAAACTATTGATGGTAATTTACTTATC (SEQ ID | | |
| | | NO: 51) | | |
| | OLEC6214 | AGCTGATAAGTAAATTACCATCAATAGTTTCTGGATATAAT | R | Cloning |
| | | AATTTAAGATTAAAAGGTAATTCTATCTTGTTGAGATCTGA | | |
| | | GCTTTCTTCTATATGATTAATATTTGCTAC (SEQ ID NO: 52) | | |
| pEC1688 | OLEC6283 | AGCTGAGATAGAATTACCTTTTAATCTC (SEQ ID NO: 53) | F | Cloning |
| | OLEC6301 | AGCTGAGATTAAAAGGTAATTCTATCTC (SEQ ID NO: 54) | R | Cloning |
| pEC1693 | OLEC6432 | GACGGCCAGTGCAGTCGAGCTCGG (SEQ ID NO: 55) | F | |
| | OLEC6433 | CCTTTTAATCTCCGCTTGCATGCCTG (SEQ ID NO: 56) | R | Mutage- |
| | | | | nesis of |
| | | | | pEC1688 |
| pEC1703 | OLEC6331 | AGCTGCGATAGAATTACCTTTTAATCTC (SEQ ID NO: 57) | F | Cloning |
| | OLEC6332 | AGCTGAGATTAAAAGGTAATTCTATCGC (SEQ ID NO: 58) | R | Cloning |
| pEC1704 | OLEC6333 | AGCTGATATAGAATTACCTTTTAATCTC (SEQ ID NO: 59) | F | Cloning |
| | OLEC6334 | AGCTGAGATTAAAAGGTAATTCTATATC (SEQ ID NO: 60) | R | Cloning |
| pEC1705 | OLEC6335 | AGCTGAGCTAGAATTACCTTTTAATCTC (SEQ ID NO: 61) | F | Cloning |
| | OLEC6336 | AGCTGAGATTAAAAGGTAATTCTAGCTC (SEQ ID NO: 62) | R | Cloning |
| pEC1706 | OLEC6337 | AGCTGAGAGAGAATTACCTTTTAATCTC (SEQ ID NO: 63) | F | Cloning |
| | OLEC6338 | AGCTGAGATTAAAAGGTAATTCTCTCTC (SEQ ID NO: 64) | R | Cloning |
| pEC1707 | OLEC6339 | AGCTGAGATCGAATTACCTTTTAATCTC (SEQ ID NO: 65) | F | Cloning |
| | OLEC6340 | AGCTGAGATTAAAAGGTAATTCGATCTC (SEQ ID NO: 66) | R | Cloning |
| pEC1708 | OLEC6341 | AGCTGAGATATAATTACCTTTTAATCTC (SEQ ID NO: 67) | F | Cloning |
| | OLEC6342 | AGCTGAGATTAAAAGGTAATTATATCTC (SEQ ID NO: 68) | R | Cloning |
| pEC1709 | OLEC6343 | AGCTGAGATAGCATTACCTTTTAATCTC (SEQ ID NO: 69) | F | Cloning |
| | OLEC6344 | AGCTGAGATTAAAAGGTAATGCTATCTC (SEQ ID NO: 70) | R | Cloning |
| pEC1710 | OLEC6345 | AGCTGAGATAGACTTACCTTTTAATCTC (SEQ ID NO: 71) | F | Cloning |
| | OLEC6346 | AGCTGAGATTAAAAGGTAAGTCTATCTC (SEQ ID NO: 72) | R | Cloning |
| pEC1711 | OLEC6347 | AGCTGAGATAGAAGTACCTTTTAATCTC (SEQ ID NO: 73) | F | Cloning |
| | OLEC6348 | AGCTGAGATTAAAAGGTACTTCTATCTC (SEQ ID NO: 74) | R | Cloning |
| pEC1712 | OLEC6349 | AGCTGAGATAGAATGACCTTTTAATCTC (SEQ ID NO: 75) | F | Cloning |
| | OLEC6350 | AGCTGAGATTAAAAGGTCATTCTATCTC (SEQ ID NO: 76) | R | Cloning |
| pEC1713 | OLEC6351 | AGCTGAGATAGAATTCCCTTTTAATCTC (SEQ ID NO: 77) | F | Cloning |
| | OLEC6352 | AGCTGAGATTAAAAGGGAATTCTATCTC (SEQ ID NO: 78) | R | Cloning |
| pEC1714 | OLEC6353 | AGCTGAGATAGAATTAACTTTTAATCTC (SEQ ID NO: 79) | F | Cloning |
| | OLEC6354 | AGCTGAGATTAAAAGTTAATTCTATCTC (SEQ ID NO: 80) | R | Cloning |
| pEC1715 | OLEC6355 | AGCTGAGATAGAATTACATTTTAATCTC (SEQ ID NO: 81) | F | Cloning |
| | OLEC6356 | AGCTGAGATTAAAATGTAATTCTATCTC (SEQ ID NO: 82) | R | Cloning |
| pEC1716 | OLEC6357 | AGCTGAGATAGAATTACCGTTTAATCTC (SEQ ID NO: 83) | F | Cloning |
| | OLEC6358 | AGCTGAGATTAAACGGTAATTCTATCTC (SEQ ID NO: 84) | R | Cloning |
| pEC1717 | OLEC6359 | AGCTGAGATAGAATTACCTGTTAATCTC (SEQ ID NO: 85) | F | Cloning |
| | OLEC6360 | AGCTGAGATTAACAGGTAATTCTATCTC (SEQ ID NO: 86) | R | Cloning |
| pEC1718 | OLEC6361 | AGCTGAGATAGAATTACCTTGTAATCTC (SEQ ID NO: 87) | F | Cloning |
| | OLEC6362 | AGCTGAGATTACAAGGTAATTCTATCTC (SEQ ID NO: 88) | R | Cloning |
| pEC1719 | OLEC6363 | AGCTGAGATAGAATTACCTTTGAATCTC (SEQ ID NO: 89) | F | Cloning |
| | OLEC6364 | AGCTGAGATTCAAAGGTAATTCTATCTC (SEQ ID NO: 90) | R | Cloning |
| pEC1720 | OLEC6365 | AGCTGAGATAGAATTACCTTTTCATCTC (SEQ ID NO: 91) | F | Cloning |
| | OLEC6366 | AGCTGAGATGAAAAGGTAATTCTATCTC (SEQ ID NO: 92) | R | Cloning |
| pEC1721 | OLEC6367 | AGCTGAGATAGAATTACCTTTTACTCTC (SEQ ID NO: 93) | F | Cloning |
| | OLEC6368 | AGCTGAGAGTAAAAGGTAATTCTATCTC (SEQ ID NO: 94) | R | Cloning |
| pEC1722 | OLEC6369 | AGCTGAGATAGAATTACCTTTTAAGCTC (SEQ ID NO: 95) | F | Cloning |
| | OLEC6370 | AGCTGAGCTTAAAAGGTAATTCTATCTC (SEQ ID NO: 96) | R | Cloning |
| pEC1723 | OLEC6371 | AGCTGAGATAGAATTACCTTTTAATATC (SEQ ID NO: 97) | F | Cloning |
| | OLEC6372 | AGCTGATATTAAAAGGTAATTCTATCTC (SEQ ID NO: 98) | R | Cloning |
| pEC1724 | OLEC6373 | AGCTGAGATAGAATTACCTTTTAATCGC (SEQ ID NO: 99) | F | Cloning |
| | OLEC6374 | AGCTGCGATTAAAAGGTAATTCTATCTC (SEQ ID NO: 100) | R | Cloning |
| pEC1725 | OLEC6375 | AGCTGCTCGAGAATTACCTTTTAATCTC (SEQ ID NO: 101) | F | Cloning |
| | OLEC6376 | AGCTGAGATTAAAAGGTAATTCTCGAGC (SEQ ID NO: 102) | R | Cloning |
| pEC1726 | OLEC6377 | AGCTGAGATAGAATTACCTTTTACGAGC (SEQ ID NO: 103) | F | Cloning |
| | OLEC6378 | AGCTGCTCGTAAAAGGTAATTCTATCTC (SEQ ID NO: 104) | R | Cloning |
| pEC1731 | OLEC6432 | GACGGCCAGTGCAGTCGAGCTCGG (SEQ ID NO: 105) | F | Mutage- |
| | OLEC6499 | CCTTTTAATCTCATCTTGCATGCCTG (SEQ ID NO: 106) | R | nesis of |
| pEC1734 | OLEC6432 | GACGGCCAGTGCAGTCGAGCTCGG (SEQ ID NO: 107) | F | pEC1688 |
| | OLEC6502 | CCTTTTAATCTCCTCTTTCATGCCTG (SEQ ID NO: 108) | R | |
| pEC1735 | OLEC6432 | GACGGCCAGTGCAGTCGAGCTCGG (SEQ ID NO: 109) | F | |
| | OLEC6515 | CCTTTTAATCTCGGCTTGCATGCCTG (SEQ ID NO: 110) | R | |

Substrates for PAM determination

| | | | |
|---|---|---|---|
| targ_wt | OLEC6503 | AGCTGTAATCATATAGAAGAAAGCTCAGATCTCAACAAGA | F |
| | | TAGAATTACCTTTTAATCTTAAATTATTATATCCAGAAACT | |
| | | ATTGATGGTAC (SEQ ID NO: 111) | |
| ntarg_wt | OLEC6504 | AGCTGTACCATCAATAGTTTCTGGATATAATAATTTAAGAT | R |
| | | TAAAAGGTAATTCTATCTTGTTGAGATCTGAGCTTTCTTCT | |
| | | ATATGATTAC (SEQ ID NO: 112) | |
| targPAM_ | OLEC6507 | AGCTGTAATCATATAGAAGAAAGCTCAGATCTCAACAAGA | F |
| A2, 3, 7T | | TAGAATTACCTTTTAATCTTTTATTTTTATATCCAGAAACTA | |
| | | TTGATGGTAC (SEQ ID NO: 113) | |
| ntargPAM_ | OLEC6508 | AGCTGTACCATCAATAGTTTCTGGATATAAAAATAAAAGA | R |
| T2, 3, 7A | | TTAAAAGGTAATTCTATCTTGTTGAGATCTGAGCTTTCTTC | |
| | | TATATGATTAC (SEQ ID NO: 114) | |
| targPAM_ | OLEC6509 | AGCTGTAATCATATAGAAGAAAGCTCAGATCTCAACAAGA | F |
| A2, 3T | | TAGAATTACCTTTTAATCTTTTATTATTATATCCAGAAACTA | |
| | | TTGATGGTAC (SEQ ID NO: 115) | |
| ntargPAM_ | OLEC6530 | AGCTGTACCATCAATAGTTTCTGGATATAATAATAAAAGAT | R |
| T2, 3A | | TAAAAGGTAATTCTATCTTGTTGAGATCTGAGCTTTCTTCT | |
| | | ATATGATTAC (SEQ ID NO: 116) | |
| targPAM_ | OLEC6531 | AGCTGTAATCATATAGAAGAAAGCTCAGATCTCAACAAGA | F |
| A2, 3G | | TAGAATTACCTTTTAATCTTGGATTATTATATCCAGAAACT | |
| | | ATTGATGGTAC (SEQ ID NO: 117) | |
| ntargPAM_ | OLEC6532 | AGCTGTACCATCAATAGTTTCTGGATATAATAATCCAAGA | R |
| T2, 3C | | TTAAAAGGTAATTCTATCTTGTTGAGATCTGAGCTTTCTTC | |
| | | TATATGATTAC (SEQ ID NO: 118) | |

Substrate for radioactive cleavage assays and electrophoretic mobility shift assays

| | | | |
|---|---|---|---|
| Psp5 | OLEC6213 | AGCTGTAGCAAATATTAATCATATAGAAGAAAGCTCAGAT | F |
| | | CTCAACAAGATAGAATTACCTTTTAATCTTAAATTATTATA | |
| | | TCCAGAAACTATTGATGGTAATTTACTTATC (SEQ ID | |
| | | NO: 119) | |
| | OLEC6214 | AGCTGATAAGTAAATTACCATCAATAGTTTCTGGATATAAT | R |
| | | AATTTAAGATTAAAAGGTAATTCTATCTTGTTGAGATCTGA | |
| | | GCTTTCTTCTATATGATTAATATTTGCTAC (SEQ ID | |
| | | NO: 120) | |

In vitro transcription of crRNA

| | | | | |
|---|---|---|---|---|
| T7 | OLEC4211 | TAATACGACTCACTATA (SEQ ID NO: 121) | F | IVT |
| promoter | | | | |
| T7- | OLEC6201 | AAAAATGACCTTCATAAATCGCTAATCTACAACAGTAGAA | R | IVT |
| crRNA4_ | | CCTATAGTGAGTCGTATTA (SEQ ID NO: 122) | | |
| rep_sp_ | | | | |
| proc | | | | |
| T7- | OLEC6202 | AGATAGAATTACCTTTTAATCTACCTATAGTGAGTCGTATT | R | IVT |
| crRNA5_ | | A (SEQ ID NO: 123) | | |
| sp | | | | |
| T7- | OLEC6203 | AGATAGAATTACCTTTTAATCTATCTACAACAGTAGAACCT | R | IVT |
| crRNA5_ | | ATAGTGAGTCGTATTA (SEQ ID NO: 124) | | |
| rep_sp_ | | | | |
| proc | | | | |
| T7- | OLEC6204 | CTCAACAAGATAGAATTACCTTTTAATCTATCTACAACAGT | R | IVT |
| crRNA5_ | | AGAAATTATTTAAAGTTCTTAGACCCTATAGTGAGTCGTAT | | |
| rep_sp_ | | TA (SEQ ID NO: 125) | | |
| full | | | | |
| T7- | OLEC6205 | ATCTACAACAGTAGAAATTATTTAAAGTTCTTAGACCTCAA | R | IVT |
| crRNA5_ | | CAAGATAGAATTACCTTTTAATCTCCTATAGTGAGTCGTAT | | |
| sp_rep_ | | TA (SEQ ID NO: 126) | | |
| full | | | | |
| T7- | OLEC6206 | ATCTACAACAGTAGAAATTATTTAAAGTTCTTAGACCTCAA | R | IVT |
| crRNA5_ | | CAAGATAGAATTACCTTTTAATCTATCTACAACAGTAGAAA | | |
| rep_sp_ | | TTATTTAAAGTTCTTAGACCCTATAGTGAGTCGTATTA | | |
| rep | | (SEQ ID NO: 127) | | |
| T7- | OLEC6318 | AGATAGAATTACCTTTTAATCTATGATGAACAGTAGAACC | R | IVT |
| crRNA5 | | TATAGTGAGTCGTATTA (SEQ ID NO: 128) | | |
| no stem | | | | |
| T7- | OLEC6440 | AGATAGAATTACCTTTTAATCTATGATGTGTTCATCAACCT | R | IVT |
| crRNA5 | | ATAGTGAGTCGTATTA (SEQ ID NO: 129) | | |
| seq mut | | | | |
| T7- | OLEC6441 | CTCAACAAGATAGAATTACCTTTTAATCTATGATGAACAGT | R | IVT |
| crRNA5 | | AGAAATTATTTAAAGTTCTTAGACCCTATAGTGAGTCGTAT | | |
| mut rep | | TA (SEQ ID NO: 130) | | |
| T7- | OLEC6422 | CTCAACAAGATAGAATTACCTTTTAATCTATGATGAACAGT | R | IVT |
| crRNA5 | | AGAAATTATTTAAAGTTCTTAGACCCTATAGTGAGTCGTAT | | |
| struct mut | | TA (SEQ ID NO: 131) | | |

Cpf1 plasmids

| | | | | |
|---|---|---|---|---|
| pEC1611 | OLEC6138 | ATGCAGGTCGACATGTCAATTTATCAAGAATTTG (SEQ ID | F | Cloning |
| | | NO: 132) | | |
| | OLEC6139 | AGCTAGCGGCCGCTTAGTTATTCCTATTCTGCACG (SEQ | R | Cloning |
| | | ID NO: 133) | | |
| pEC1701 | OLEC6287 | ATGCAGGGTACCATGTCAATTTATCAAGAATTTG (SEQ ID | F | Cloning |
| | | NO: 134) | | |
| | OLEC6288 | AGCTACGGCCGTTAGTTATTCCTATTCTGCACG (SEQ ID | R | Cloning |
| | | NO: 135) | | |

Cpf1 mutagenesis

| | | | | |
|---|---|---|---|---|
| pEC1776 | OLEC6409 | TCGTAAACAATCAATACCTAAAAAAATCACTGCCCCAGCT | F | |
| | | AAAGAGGCA (SEQ ID NO: 136) | | |
| | OLEC6410 | TGCCTCTTTAGCTGGGGCAGTGATTTTTTTAGGTATTGAT | R | |
| | | TGTTTACGA (SEQ ID NO: 137) | | |
| pEC1777 | OLEC6561 | CTCTTTTTTAGGATTATCTTTGTTTGCATTAGCTATTGCCT | F | |
| | | CTTTAGCTGGGT (SEQ ID NO: 138) | | |
| | OLEC6562 | ACCCAGCTAAAGAGGCAATAGCTAATGCAAACAAAGATA | R | |
| | | ATCCTAAAAAAGAG (SEQ ID NO: 139) | | |
| pEC1778 | OLEC6563 | GAAAAACTTATCTTCAGTAAAGCGTTTATCTGCGATTAAAT | F | |
| | | CATATTCAAAAACACTCTCTTTT (SEQ ID NO: 140) | | |
| | OLEC6564 | AAAAGAGAGTGTTTTTGAATATGATTTAATCGCAGATAAA | R | |
| | | CGCTTTACTGAAGATAAGTTTTTC (SEQ ID NO: 141) | | |
| pEC1779 | OLEC6565 | GGACAGTGAAAGAAAAACTTATCTTCAGTAGCGCGTTTAT | F | |
| | | CTTTGATTAAATCATATTCAAA (SEQ ID NO: 142) | | |
| | OLEC6566 | TTTGAATATGATTTAATCAAAGATAAACGCGCTACTGAAG | R | |
| | | ATAAGTTTTTCTTTCACTGTCC (SEQ ID NO: 143) | | |
| pEC1782 | OLEC6444 | AAGCTAAATGTCTTTCACCTCTAGCTATACTTAATATATGA | F | |
| | | ACATCATTTGCT (SEQ ID NO: 144) | | |
| | OLEC6445 | AGCAAATGATGTTCATATATTAAGTATAGCTAGAGGTGAA | R | |
| | | AGACATTTAGCTT (SEQ ID NO: 145) | | |
| pEC1783 | OLEC6476 | TCATATATTAAGTATAGATAGAGGTGCAAGACATTTAGCT | F | |
| | | TACTATACTTTGG (SEQ ID NO: 146) | | |
| | OLEC6477 | CCAAAGTATAGTAAGCTAAATGTCTTGCACCTCTATCTAT | R | |
| | | ACTTAATATATGA (SEQ ID NO: 147) | | |
| pEC1784 | OLEC6411 | CCATCTACCAAAGTATAGTAAGCTAAAGCTCTTTCACCTC | F | Mutage- |
| | | TATCTATACTTAATAT (SEQ ID NO: 148) | | nesis of |
| | | | | pEC1611 |
| | OLEC6412 | ATATTAAGTATAGATAGAGGTGAAAGAGCTTTAGCTTACT | R | |
| | | ATACTTTGGTAGATGG (SEQ ID NO: 149) | | |
| pEC1785 | OLEC6464 | CCTTTACCATCTACCAAAGTATAGGCAGCTAAATGTCTTT | F | |
| | | CACCTCTATC (SEQ ID NO: 150) | | |
| | OLEC6465 | GATAGAGGTGAAAGACATTTAGCTGCCTATACTTTGGTAG | R | |
| | | ATGGTAAAGG (SEQ ID NO: 151) | | |
| pEC1788 | OLEC6446 | CTCTTTTAAATCCAAAATTTAAATCCGCAAAAACCACAATA | F | |
| | | GCATTATACTCTATAACT (SEQ ID NO: 152) | | |
| | OLEC6447 | AGTTATAGAGTATAATGCTATTGTGGTTTTTGCGGATTTAA | R | |
| | | ATTTTGGATTTAAAAGAG (SEQ ID NO: 153) | | |
| pEC1790 | OLEC6472 | AATTAGCATTTTTTCTAACTTTTGAGCGACCTGCTTCTCTA | F | |
| | | CCTTGAAACG (SEQ ID NO: 154) | | |
| | OLEC6473 | CGTTTCAAGGTAGAGAAGCAGGTCGCTCAAAAGTTAGAA | R | |
| | | AAAATGCTAATT (SEQ ID NO: 155) | | |
| pEC1791 | OLEC6448 | GTTTAGTTTCTCAATTAGCATTTTTGCTAACTTTTGATAGA | F | |
| | | CCTGCTTCTC (SEQ ID NO: 156) | | |
| | OLEC6449 | GAGAAGCAGGTCTATCAAAAGTTAGCAAAAATGCTAATTG | R | |
| | | AGAAACTAAAC (SEQ ID NO: 157) | | |
| pEC1796 | OLEC6419 | CTGCTACTGGTGAAATTAGATAAGCTAACTCAGTACCTGT | F | |
| | | TTTTGAG (SEQ ID NO: 158) | | |
| | OLEC6420 | CTCAAAAACAGGTACTGAGTTAGCTTATCTAATTTCACCA | R | |
| | | GTAGCAG (SEQ ID NO: 159) | | |
| pEC1799 | OLEC6450 | GATAAGCACCATTGGCAGCAGCATCTTGAGGCATA (SEQ | F | |
| | | ID NO: 160) | | |
| | OLEC6451 | TATGCCTCAAGATGCTGCTGCCAATGGTGCTTATC (SEQ | R | |
| | | ID NO: 161) | | |

Probes for Northern blot analysis of pre-crRNA processing

| | | | |
|---|---|---|---|
| spacer | OLE06528 | ATCAAGCCCTTCATGCGCTTCAAGGTGCA (SEQ ID | R |
| | | NO: 162) | |

Size markers for radioactive RNA cleavage assays

| | | |
|---|---|---|
| 37 nt | OLEC5951 | AGTTTAGGTACCTTATTTTCTCCACTCTAAACTTGAT (SEQ |
| | | ID NO: 163) |
| 47 nt | OLEC6260 | ATATTCAACATATTGACCGGCCTGCAGAGTAAGGATGTTG |
| | | GGTCTAC (SEQ ID NO: 164) |
| 54 nt | OLEC6441 | CTCAACAAGATAGAATTACCTTTTAATCTATGATGAACAGT |
| | | AGAAATTATTTAAAGTTCTTAGACCCTATAGTGAGTCGTAT |
| | | TA (SEQ ID NO: 165) |
| 60 nt | OLEC6489 | ATGGGCCATCATCATCATCATCATCATCATCATCACACTA |
| | | CAGTAAAAAAAAACAGAGCG (SEQ ID NO: 166) |

Plasmid sequencing anaysis

| | | | | |
|---|---|---|---|---|
| proto- | pUCM13- | FROM SEQLAB | F | SEQ |
| spacer | 52 | | | |
| | pUCM13- | FROM SEQLAB | R | SEQ |
| | rev-157 | | | |
| Plasmids | T7 prom | FROM SEQLAB | F | SEQ |
| containing | T7 term | FROM SEQLAB | R | SEQ |
| Cpf1 or | OLEC6482 | GGTGGTAAATTTG (SEQ ID NO: 167) | F | SEQ |
| a variant | OLEC6483 | GTCAGTCAGAAG (SEQ ID NO: 168) | F | SEQ |
| thereof | OLEC6498 | GGTTTATAAGCTAAATGGTGAGGC (SEQ ID NO: 169) | F | SEQ |
| pEC1690 | OLEC6319 | GTCGCGAACGCCAGCAAG (SEQ ID NO: 170) | R | SEQ |

DNA insert for crRNA cloning

| | | | |
|---|---|---|---|
| pEC1690 | IDT gBlock | ATGCAGAAGCTTTTGACAGCTAGCTCAGTCCTAGGTATAA | Cloning |
| | | TGCTAGCGTCTAAGAACTTTAAATAATTTCTACTGTTGTAG | |
| | | ATTGCACCTTGAAGCGCATGAAGGGCTTGATGTCTAAGA | |
| | | ACTTTAAATAATTTGTCTGTATATTATTGATTTCTAAATTAG | |
| | | AATTTTCGGCCGATGCAG (SEQ ID NO: 171) | |

While the present invention has been described in terms of specific embodiments, it is understood that variations and modifications will occur to those skilled in the art. Accordingly, only such limitations as appear in the claims should be placed on the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

1. A method for targeting, editing, modifying, or manipulating a target DNA at one or more locations in a cell or in vitro, the method comprising:
i) introducing a heterologous Cpf1 polypeptide or a nucleic acid encoding a Cpf1 polypeptide into the cell;
ii) introducing a) a single heterologous guide RNA (gRNA) or a DNA encoding the same; said gRNA comprising a precursor CRISPR RNAs (pre-crRNA) encoding one or more crRNAs or one or more intermediate or mature crRNAs, each guide RNA comprising at a minimum a repeat-spacer in the 5 to 3 direction, wherein the repeat comprises a stem-loop structure and the spacer comprises a DNA-targeting segment complementary to a target sequence in the target DNA: and
iii) creating one or more cuts in the target DNA, wherein DNA cleavage is mediated by the Cpf1 polypeptide DNase, or otherwise targeting or manipulating the target DNA;
wherein the Cpf1 polypeptide is directed to the target DNA by the gRNA in its processed or unprocessed form.

2. The method of claim 1, wherein gRNA is cleaved by RNase activity of the Cpf1 polypeptide into one or more mature crRNAs, each comprising at least one repeat and at least one spacer.

3. The method of claim 1, wherein gRNA contains one or more repeat-spacer directing the Cpf1 polypeptides to two or more distinct sites in the target DNA.

4. The method of claim 1, wherein each cut in the target DNA is double-stranded and contains a 5′ overhang.

5. The method of claim 4, wherein the 5′ overhang contains five nucleotides.

6. The method of claim 4, wherein at least two 5′ overhangs are created, each being non-homologues or non-complementary to each other so as to reduce the likelihood of chromosomal translocations caused by the rejoining or reannealing of heterologous cleavage sites.

7. The method of claim 1, further comprising allowing the cuts in the target DNA to be repaired by endogenous DNA polymerase repair mechanism present in the cell.

8. The method of claim 1, further comprising introducing a donor DNA sequence under conditions that allow editing of the target DNA by homology directed repair.

9. The method of claim 1, wherein the Cpf1 polypeptide is expressed as a monomer.

10. The method of claim 1, wherein the Cpf1 polypeptide has a calculated molecular weight of about 153 kDa or an apparent molecular weight of about 187 kDa.

11. The method of claim 1, wherein the Cpf1 polypeptide has an RNA cleavage domain and a DNA cleavage domain.

12. The method of claim 1, wherein the RNase activity of the Cpf1 polypeptide cleaves gRNAs within the repeat of the repeat-spacer array.

13. The method of claim 12, wherein the Cpf1 polypeptide cleaves gRNA four nucleotides upstream of the stem-loop structure in the array.

14. The method of claim 1, wherein RNase activity of the Cpf1 polypeptide requires Mg²⁺.

15. The method of claim 1, wherein the gRNA is cleaved and processed into one or more intermediate crRNAs, which are subsequently processed into one or more mature crRNAs.

16. The method of claim 1, wherein DNase activity of the Cpf1 polypeptide requires Mg²⁺, Mn²⁺, or Ca²⁺.

17. The method of claim 1, wherein the Cpf1 polypeptide recognizes a PAM sequence in the target DNA, said PAM sequence being 5′-YTN-3′ (wherein Y is T or C) upstream of the crRNA-complementary DNA sequence on the non-target strand.

18. The method of claim 17, wherein the gRNA has a seed sequence of eight nucleotides, located at the 5′ end of the spacer, and is proximal to the PAM sequence on the target DNA.

19. The method of claim 17, wherein Cpf1 polypeptide cleaves the target DNA about 20 nucleotides upstream of the PAM sequence.

20. The method of claim 17, wherein Cpf1 polypeptide cleaves the DNA exactly 22 base pairs upstream of the PAM sequence on the crRNA-complementary target strand and 17 base pairs downstream of the PAM sequence on the non-crRNA-complementary non-target strand.

21. The method of claim 1, wherein the gRNA comprises several nucleotides upstream of the stem-loop thereby enhancing DNase activity of the Cpf1 polypeptide.

22. The method of claim 1, wherein the Cpf1 polypeptide is mutated a) to reduce or eliminate RNase activity, while maintaining DNase activity or b) to reduce or eliminate DNase activity, while maintaining RNase activity.

23. The method of claim 17, wherein modification of specific amino acid residues in the Cpf1 polypeptide is selected from the group consisting of: H843, K852, K869, F873, D917, E1006, D1255, E920, Y1024, D1227, E1028, H922, and Y925.

24. The method of claim 1, wherein the Cpf1 polypeptides is mutated to a) reduce cleavage of one, but not the other, DNA strand in the target DNA, b) to increase RNA stability and/or c) to increase DNA binding.

25. The method of claim 1, wherein the Cpf1 polypeptide is a mutant polypeptide with altered Cpf1 endoribonuclease activity or associated half life of pre-crRNA, intermediate crRNA, or mature crRNA, and having one or more mutations at amino acid residues selected from the group consisting of: H843, K852, K869, and F873.

26. The method of claim 1, wherein the Cpf1 polypeptide is a mutant polypeptide with altered or abrogated DNA endonuclease activity without substantially diminished or enhanced endoribonuclease activity or binding affinity to DNA, and having one or more mutations at amino acid residues selected from the group consisting of: D917, E1006, and D1255.

27. The method of claim 1, wherein the Cpf1 polypeptide is a mutant polypeptide with no DNA endonuclease activity in the presence of Ca²⁺, without substantially diminished or enhanced DNA endonuclease activity in the presence of Mg²⁺, and having one or more mutations at amino acid residues selected from the group consisting of: E920, Y1024, and D1227.

28. The method of claim 1, wherein the Cpf1 polypeptide is a mutant polypeptide with no DNA endonuclease activity in the presence of Ca²⁺, and substantially reduced DNA endonuclease activity of the non-target strand in the presence of Mg²⁺, and having a mutation at amino acid residue E1028.

29. The method of claim 1, wherein the Cpf1 polypeptide is a mutant polypeptide with substantially decreased DNA endonuclease activity of the target strand in the presence of Ca²⁺, without substantially diminished or enhanced DNA endonuclease activity in the presence of Mg²⁺, and having one or more mutations at amino acid residues selected from: H922 and Y925.

30. The method of claim 1, wherein the cell is a bacterial cell, a fungal cell, an archaea cell, a plant cell, or an animal cell.

31. The method of claim 1, wherein the Cpf1 polypeptide and the gRNA are introduced into the cell by the same or different recombinant vectors encoding the polypeptide and the gRNA.

32. The method of claim 1, wherein the Cpf1 polypeptide is from the species selected from the group consisting of: F. novicida U112, Prevotella albensis, Acidaminococcus sp. BV3L6, Eubacterium eligens CAG:72, Butyrivibrio fibrisolvens, Smithella sp. SCADC, Flavobacterium sp. 316, Porphyromonas crevioricanis and Bacteroidetes oral taxon 274.

33. The method of claim 1, wherein pre-crRNA or intermediate crRNA are processed into mature crRNA by a Cpf1 polypeptide, thereby the mature crRNA becomes available for directing the Cpf1 DNA endonuclease activity.

34. The method of claim 33, wherein the Cpf1 polypeptide is more readily complexed with the mature crRNA as a result of being processed by the Cpf1 polypeptide.

35. The method of claim 34, wherein the Cpf1 polypeptide is able to cleave, isolate or purify one or more mature crRNAs from the gRNA which further comprises a heterologous sequence incorporated 5′ or 3′ to one or more crRNA sequences within the gRNA oligonucleotide or its DNA expression construct.

36. The method of claim 1, wherein heterologous sequences are incorporated into gRNA to modify the stability, half-life, expression level thereof or timing of interaction with the Cpf1 polypeptide or target DNA.

37. The method of claim 1, wherein the pre-crRNA sequence is modified so as to provide for differential regulation of two or more mature crRNA sequences within the pre-crRNA sequence.

38. The method of claim 1, wherein the Cpf1 polypeptide or gRNA moiety is linked to a dimeric FOK1 nuclease, a nickase, a temperature sensitive variant thereof, or another polypeptide having endonuclease activity, thereby being directed to one or more DNA target.

39. The method of claim 38, wherein the Cpf1 polypeptide linked with a dimeric FOK1 nuclease is introduced into the cell together with the single gRNA (either as RNA or encoded as DNA), both under the control of one promoter, and wherein the Cpf1 polypeptide cleaves pre-crRNAs upstream of the stem-loop structures to generate two or more intermediate crRNAs.

40. The method of claim 1, wherein the Cpf1 polypeptide or gRNA moiety is linked to a single or double strand DNA donor template, thereby facilitating homologous recombination of exogenous DNA sequences, as directed by gRNA to one or more sites on the target DNA.

41. The method of claim 40, wherein the donor template is cleaved from the gRNA by the Cpf1 polypeptide, thus facilitating homologous recombination or homology directed repair.

42. The method of claim 40, wherein the donor template the donor template remains linked to gRNA while the Cpf1 polypeptide cleaves gRNA to liberate intermediate or mature crRNAs.

43. The method of claim 1, wherein the Cpf1 polypeptide or the gRNA is linked to a transcriptional activator or repressor, or epigenetic modifier so as to detect one or more DNA target sites or to modulate signaling or expression associated with the sites.

44. The method of claim 43, wherein the epigenetic modifier is a methylase, a demethylase, an acetylase, or a deacetylase.

45. The method of claim 1, wherein the target DNA is double stranded target and wherein the Cpf1 polypeptide possesses no or reduced endonuclease activity against ssRNA, dsRNA, or heteroduplexes of RNA and DNA.

46. A system for targeting, editing, modifying, or manipulating target DNA in vitro or in a cell, the composition comprising a heterologous vector encoding or providing a Cpf1 polypeptide and a single heterologous guide nucleic acid comprising apre-crRNA or one or more intermediate or mature crRNAs, each pre-crRNA or intermediate or mature crRNAs, comprising at a minimum a repeat-spacer in the 5′ to 3′ direction, wherein the repeat comprises a stem-loop structure and the spacer comprises a DNA-targeting segment.

47. The system of claim 46, wherein the system further comprises a buffer providing Mg²⁺ or Ca²⁺, or both.

48. The system of claim 46, wherein guide nucleic acid has a seed sequence of eight nucleotides proximal to the stem-loop structure, said seed sequence being fully complementary to a sequence in the target DNA.

49. The system of claim 48, wherein the complementary sequence in the target DNA is immediately upstream of a PAM sequence, the PAM sequence being 5′-YTN-3′ (wherein Y is T or C) located on the “non-target” strand.

50. The system of claim 46, wherein the Cpf1 polypeptide is mutated.

51. The system of claim 50, wherein the mutation in the Cpf1 polypeptide selected from the group consisting of: H843, K852, K869, F873, D917, E1006, D1255, E920, Y1024, D1227, E1028, H922, and Y925.

52. The system of claim 46, wherein the system further comprises a donor DNA sequence for editing the target DNA sequence by homology directed repair.

53. The system of claim 46, wherein the Cpf1 polypeptide is from the species selected from the group consisting of: F. novicida U112, Prevotella albensis, Acidaminococcus sp. BV3L6, Eubacterium eligens CAG:72, Butyrivibrio fibrisolvens, Smithella sp. SCADC, Flavobacterium sp. 316, Porphyromonas crevioricanis and Bacteroidetes oral taxon 274.

54. A composition for editing or modifying DNA at one or more locations in a cell consisting essentially of:
i) a Cpf1 polypeptide or a nucleic acid encoding a Cpf1 polypeptide; and/or
ii) a single heterologous nucleic acid (gRNA) comprising at least one pre-crRNAs or intermediate or mature crRNAs, each guide RNA comprising at a minimum a repeat-spacer in the 5′ to 3′ direction, wherein the repeat comprises a stem-loop structure and the spacer comprises a DNA-targeting segment complementary to a target sequence in the target DNA.

55. A composition of claim 54 for editing or modifying DNA at multiple locations in a cell consisting essentially of:
i) a Cpf1 polypeptide or a nucleic acid encoding a Cpf1 polypeptide; and/or
ii) a single heterologous nucleic acid (gRNA) comprising a pre-crRNAs or two or more intermediate or mature crRNAs, each guide RNA comprising at a minimum a repeat-spacer in the 5′ to 3′ direction, wherein the repeat comprises a stem-loop structure and the spacer comprises a DNA-targeting segment complementary to a target sequence in the target DNA.

56. The composition of claim 54, wherein the composition further comprises iii) a polynucleotide donor template.

57. The composition of claim 55, wherein guide RNA is linked to a donor template nucleic acid.
